(19) United States
(12) Patent Application Publication
(10) Pub. No.: US 2023/0173071 A1
(43) Pub. Date: Jun. 8, 2023

(54) TREATMENT OF MYOPIA AND OTHER OCULAR CONDITIONS USING SINGLET OXYGEN GENERATED FROM DYES ACTIVATED BY NEAR-INFRARED LIGHT

(71) Applicants: California Institute of Technology, Pasadena, CA (US); The Regents Of the University of California, Oakland, CA (US); U.S. Government represented by the Development of Veterans Affairs, Washington, DC (US)

(72) Inventors: John B. JARMAN, Pasadena, CA (US); Dennis A. DOUGHERTY, Pasadena, CA (US); Robert H. GRUBBS, South Pasadena, CA (US); Christopher B. MAROTTA, Valley Village, CA (US); Daniel M. SCHWARTZ, San Francisco, CA (US)

(21) Appl. No.: 18/082,812

(22) Filed: Dec. 16, 2022

Related U.S. Application Data
(63) Continuation of application No. 16/661,644, filed on Oct. 23, 2019, now abandoned.
(60) Provisional application No. 62/750,095, filed on Oct. 24, 2018.
(60) Provisional application No. 62/797,068, filed on Jan. 25, 2019.
(60) Provisional application No. 62/877,101, filed on Jul. 22, 2019.

(51) Int. Cl. A61K 41/00; A61P 27/02

(52) U.S. Cl. CPC A61K 41/0057; A61P 27/02

(57) ABSTRACT

This current disclosure is directed to compositions based on certain heptamethine dyes useful for generating singlet oxygen using NIR radiation, optionally comprising additives and solvents that enhance the performance of these dyes, and procedures using these compositions to modify treat myopia and other ocular conditions. In some cases, the methods use near-infrared irradiation to improve the mechanical strength of the sclera.

### CROSS REFERENCE TO RELATED APPLICATIONS

This application claims priority to U.S. application Ser. No. 62/750,095, filed Oct. 24, 2018, and Ser. No. 62/797,068, filed Jan. 25, 2019, and Ser. No. 62/877,101, filed Jul. 22, 2019, the contents of which are incorporated by reference herein for all purposes.

### TECHNICAL FIELD

This invention relates methods to treat myopia and other ocular conditions and associated compositions. In some cases, the methods use near-infrared irradiation to improve the mechanical strength of the sclera.

The compositions are based on certain heptamethine dyes useful for generating singlet oxygen using NIR radiation. In addition to the use of these dyes, the compositions comprise additives that enhance the performance of these dyes for this purpose.

### BACKGROUND

Myopia is a rapidly growing problem throughout Asia, particularly in China, Japan, Korea, Singapore, and Taiwan, where it is reaching epidemic proportions. The prevalence of myopia continues to rise globally; it can be estimated that over 22% of the world's population, or 1.5 billion people, are myopic but by the year 2050, it's expected that roughly half the people on the planet will be myopic. That is, with current population projections, it is projected that myopia will affect nearly 5 billion people by the year 2050. Asian countries are particularly affected, with myopia levels reaching 70-80% in east Asian countries. Recent studies of males 15 to 24 years old in Japan, Singapore, Taiwan, Korea, and China showed incidences of 59%, 82%, 86%, 96.5%, and 95.5% respectively. Furthermore, cases of high myopia—a more severe form of myopia associated with greater vision impairment and a higher likelihood of ocular complications—are on the rise. Currently, 4% of the global population is affected by high myopia, a number that is projected to more than double by 2050. Again, studies of east Asian young male populations have shown levels of 14 to 21% for this condition.

While most myopia is treatable with refractive correction, some patients with high myopia (>8 diopters) develop degenerative changes in the macula that cause central visual loss. These degenerative changes are not treatable with eyeglasses, contact lenses, or refractive corneal surgery (LASIK). Highly myopic eyes that succumb to degenerative myopia develop progressive scleral thinning and stretching of chorioretinal tissues leading to an outpouching (staphyloma) in the region of the posterior pole. While a staphyloma might develop in the fourth or fifth decade of life, often visual loss occurs 10-20 years later. Indeed, degenerative myopia is the leading cause of visual loss in many Asian countries. Degenerative myopia is a progressive disease that poses a significant risk to vision. It is currently the leading cause of central vision loss in Asia and a steadily growing problem; projections suggest that the number of worldwide degenerative myopia cases will double by 2050.

Degenerative myopia is often associated with scleral thinning and stretching, the causes of which are not completely understood, but reduction in the mechanical strength of the sclera is a contributory factor. Sufficiently increasing the tensile strength, or modulus, of the sclera would prevent ocular enlargement and reduce progression of myopia. Such a therapy will be useful not only in patients with incipient degenerative myopia, but also in patients with early onset myopia to prevent progression to higher magnitude refractive errors. At present, there is no effective therapy to retard the progressive ocular axial elongation and scleral thinning that characterize the development of degenerative myopia. Although refractive myopia can be corrected through optical measures, the stretching of the macular sclera in patients with high degrees of myopia can lead to severe visual loss from retinal atrophy and/or choroidal neovascluarization. In fact, in countries with a high prevalence of myopia, myopic macular degeneration is one of the leading causes of blindness. Currently, no treatment is available that addresses the root of the disorder: the progressive expansion of the eyeball's axial length due to thinning of the sclera.

Given the limitations of current therapies for treating myopia, new therapies without such limitations are needed. The present invention addresses at least some aspects of this need. This disclosure is directed to taking advantage of the discoveries cited herein to avoid at least some of the problems associated with previously known methods.

### SUMMARY

The present disclosure is directed to methods for strengthening ocular tissue, for example by in situ polymerization or crosslinking of the tissues of the ocular tissue, especially the sclera, and the compositions which allow for these methods.

The disclosure is also directed, at least in part, to the localized generation of singlet oxygen in the eye for the treatment of bacterial and fungal infections, and tumors (e.g., photodynamic therapy).

Some of these embodiments include methods for using and modifying one of the disclosed compositions to alter at least one mechanical and/or chemical property of a tissue in a patient directly by irradiating one of the disclosed photoactive compositions with near-infrared (NIR) light, wherein the photoactive composition is preferably adjacent to or contacts or has permeated the tissue. In such embodiments, the mechanical and/or chemical property being altered can be tensile strength, compression strength, flexural strength, modulus, elongation, toughness of the tissue, or a combination of two or more of these properties.

In some aspects, the tissue is generally an ocular tissue, and may be at least a portion a sclera and/or a portion of a lamina cribrosa. In some aspects, the methods further comprise administering the photoactive composition directly to the tissue of the patient. This may be done either topically or by injection. Where the tissue is an ocular tissue, the photoactive composition may be administered directly to the tissue by retrobulbar injection.

In some aspects of the methods described herein, the patient has or is at risk of developing an ocular deformation condition comprising one or more of degenerative myopia, regular myopia, scleral staphyloma, keratoconus, or glaucoma. For such patients, the methods may be applied to address, either prevent or inhibit further progression of the condition. In other aspects, the methods may be used in treating other conditions, such as infections or tumors, where the singlet oxygen is deleterious to the bacteria, fungi, or tumors.

In particular, the present disclosure is directed to the use of dyes that absorb near-infrared (NIR) light and subsequently generate singlet oxygen.

Singlet oxygen—the first excited state of O₂—has significant therapeutic potential. It is already used in a variety of clinical applications, from photodynamic therapy to corneal crosslinking, and new applications continue to emerge. For most therapeutic applications, singlet oxygen is generated in situ via excitation of the dyes. Compared to visible light, NIR light offers several advantages, most notably in the present application, a minimally invasive method that provides irradiation through the pupil, without significantly harming the eye. There are few small molecule NIR chromophores capable of generating singlet oxygen past 800 nm, and those that can do not absorb significantly above 800 nm. In fact, no single photon chemistry has previously been observed above 900 nm. Different methods—such as two-photon excitation and upconverting nanoparticles—have been proposed to circumvent this issue, but a need remains for small molecules capable of directly generating singlet oxygen using NIR light.

The present inventors demonstrated the utility of this approach in eye expansion models using a formulation consisting of an FDA approved dye (indocyanine green, ICG), an additive that increases dye solubility (e.g., surfactants such as benzalkonium chloride and/or salts, such as sodium iodide), and a solvent that increases the lifetime of singlet oxygen (deuterated water). Although this formulation is robust, permutations on the above formulation are also expected to give corneal and scleral crosslinking, and this disclosure captures other dyes believed to be suitable for this purpose. The treatment disclosed herein is minimally invasive due to the use of NIR light to induce scleral cross-linking (FIG. 1 and FIG. 2), and represents a new way to treat degenerative myopia, a disease whose burden continues to grow globally.

The present disclosure sets forth methods of altering a mechanical and/or chemical property of a tissue, preferably a collagen-containing tissue, optionally in a patient, each method comprising irradiating a near-infrared-(NIR) photoactive direct treatment composition with near-infrared light in the presence of oxygen.

The present disclosure sets forth methods of treating diseases, for example, infections or tumors, each method comprising irradiating a near-infrared-(NIR) photoactive direct treatment composition with near-infrared light in the presence of oxygen.

In certain aspects of these methods, the near-infrared (NIR) photoactive direct treatment composition comprises a near-infrared dye that generates singlet oxygen when irradiated with near-infrared light in the presence of oxygen;

In certain aspects of these methods, the near-infrared (NIR) photoactive direct treatment composition is adjacent to (contacts) or has permeated the tissue; and

In certain aspects of these methods, the irradiating results in a change in the mechanical and/or chemical property of a tissue in the patient or in the treatment of the disease.

In certain aspects of these methods, the mechanical and/or chemical property is tensile strength, compression strength, flexural strength, modulus, elongation, or toughness of the tissue.

In certain aspects of these methods, the tissue is an ocular tissue.

In certain independent aspects of these methods, the ocular tissue includes at least a portion of a cornea, a sclera, or a lamina cribrosa.

In certain independent aspects of these methods, the patient has or is at risk of developing an ocular condition comprising one or more of degenerative myopia, regular myopia, scleral staphyloma, keratoconus, or glaucoma. In other aspects, the methods are used in the treatment of keratoconus and other ectatic corneal conditions and corneal infections (infectious keratitis), and ocular tumors.

In certain aspects of these methods, the patient further comprising administering the near-infrared (NIR) photoactive direct treatment composition to the tissue, either topically or by injection.

In certain aspects of these methods, the near-infrared (NIR) absorbing dye comprises a cyanine structure, a pyrylium structure, or a thiopyrylium structure, or a combination thereof. The disclosure sets forth a more complete recitation of options than are contained in this section. Each of the types of dyes and their substituents and substitution patterns are considered alternative aspects of the present disclosure

Alternatively, or additionally, in certain aspects of these methods, the near-infrared (NIR) absorbing dye comprises a structure of:

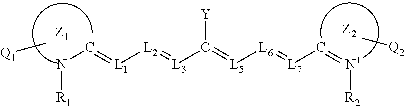

wherein

L₁, L₂, L₃, L₅, L₆, and L₇ are substituted or unsubstituted methines, wherein the optional substituents are independently C₁₋₆ alkyl or C₂₋₆ alkenyl; or L₁ and L₃, or L₃ and L₅, or L₅ and L₇ may be linked with a C₂₋₄ alkylene or C₂₋₄ alkenylene substituent to form a 5- to 7-membered ring;

each of Z¹ and Z² is independently a five- or six-membered nitrogen-containing heterocyclic ring, optionally fused to another aryl or heteroaryl ring;

each of Q₁ and Q₂ is independently H or a substituent positioned on the five- or six-membered nitrogen-containing heterocyclic ring and/or the optionally fused aryl or heteroaryl ring, each optional substituent comprising an optionally substituted C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof, —(C₀₋₁₂alkyl)borate,

R₁ and R₂ is independently C₁₋₁₂ alkyl, —[CH₂—CH₂—O—]₁₋₆R¹⁰, —(C₀₋₁₂alkyl)amino acid residue, or a 5- or 6-member ringed aryl or heteroaryl, each of which may be optionally substituted with one or more —(C₀₋₁₂alkyl)(SO₃)—R¹⁰ or a salt thereof, —(C₀₋₁₂alkyl)(SO₄)—R¹⁰ or a salt thereof, —(C₀₋₁₂alkyl)(PO₄)—R¹⁰ or a salt thereof, —(C₀₋₁₂alkyl)OR¹⁰, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof or —(C₀₋₁₂alkyl)borate or borate ester;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substitutedaryloxy, optionally substituted heteroaryloxy, halogen, or optionally substituted cationic heteroaryl moiety.

Alternatively, or additionally, Z₁ and Z₂ independently comprise a pyrrole ring, imidazole ring, isothiazole ring, isoxazole ring, oxadiazole ring, oxazole ring, pyrazole ring, pyrimidyl, thiazole ring, selenazole ring, thiadiazole ring, triazole ring, or a pyridine ring, each independently and optionally fused to a phenyl, naphthyl, pyridinyl, quinolinyl, quinoxalinyl, N-alkyl-benzoindolenine, dibenzofuranyl, or dibenzothiophenyl.

Other srecific permutation and descriptions for the variables, including L₁, L₂, L₃, L₅, L₆, and L₇, Z₁ and Z₂, Q₁ and Q₂, R₁ and R₂, R¹⁰, and Y are set forth elsewhere herein.

Alternatively, or additionally, in some aspects, the near-infrared (NIR) absorbing dye comprises a structure or rotational or conformation isomer of:

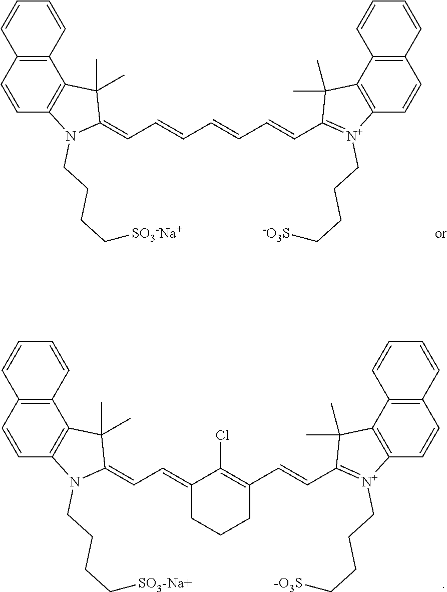

Alternatively, or additionally, the near-infrared (NIR) absorbing dye comprises a structure of:

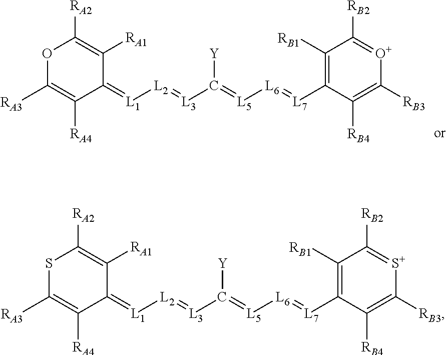

respectively, wherein

L₁, L₂, L₃, L₅, L₆, and L₇ are substituted or unsubstituted methines, wherein the optional substitutents are independently C₁₋₆ alkyl or C₂₋₆ alkenyl; or L₁ and L₃, or L₃ and L₅, or L₅ and L₇ may be linked with C₂₋₄ alkylene or C₂₋₄ alkenylene substituents;

R_{A1}, R_{A2}, R_{A3}, R_{A4}, R_{B1}, R_{B2}, R_{B3}, and R_{B4} are each independently H, deutrium, or tritium, an C₁₋₁₂ alkyl, —[CH₂—CH₂—O—]₁₋₆ R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂ or a salt thereof, —(C₀₋₁₂alkyl)N(R¹⁰)₂ or a salt thereof, —(C₀₋₁₂alkyl)borate;

n is independently 0, 1, 2, 3, or 4, preferably 2;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substituted aryloxy, optionally substituted heteroaryloxy, halogen, or optionally substituted cationic nitrogen-containing heteroaryl moiety. Other srecific permutation and descriptions for the variables, including L₁, L₂, L₃, L₅, L₆, and L₇, R_{A1}, R_{A2}, R_{A3}, R_{A4}, R_{B1}, R_{B2}, R_{B3}, and R_{B4}, n, R¹⁰, and Y, are set forth elsewhere herein.

In certain aspects of the methods, the irradiating is done with a light having a wavelength in a range of from 750 nm to 1400 nm, or any of the ranges defined herein.

Alternatively or additionally, the near-infrared (NIR) photoactive direct treatment composition further comprises or is associated with a biocompatible solvent that:

(a) is optically transparent in the UV-VIS and near-infrared range of the optical spectrum;

(b) provides an oxygen solubility greater than H₂O under comparable oxygen partial pressures, preferably a fluorinated or perfluorinated solvent;

(c) comprises an additive that provides a solubility of the near-infrared (NIR) absorbing dye in that solvent that is higher than the solubility of the near-infrared (NIR) absorbing dye in the absence of the additive, preferably a surfactant or alkali metal salt, (d) is or comprises a deuterated solvent, preferably D₂O;

(e) is oxygenated before or during the irradiation, preferably such that the dissolved oxygen is at a level greater than the equilibrium concentration of oxygen of the composition with ambient atmospheric air; or

(f) a combination of two or more of (a)-(e).

Alternatively or additionally, the near-infrared (NIR) photoactive direct treatment composition further comprises an additive that enhances the solubility of the near-infrared dye, preferably surfactant or alkali metal salt, preferably independently present at a level in a range from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the direct treatment composition.

In certain other aspects, the disclosure sets forth compositions useful for use in the methods set forth herein. For example, in some aspects, such compositions comprise:

(a) a compound comprising a near-infrared (NIR) absorbing dye that generates singlet oxygen, when irradiated with light in the presence of oxygen at a wavelength in a range of from 800 nm to 1400 nm, or in a range comprising two of more of these foregoing ranges; and

(b) one or more of
(i) an optically transparent, biocompatible solvent
(ii) a biocompatible solvent having an oxygen solubility that is greater than the oxygen solubility in H₂O under comparable oxygen partial pressures, preferably a fluorinated or perfluorinated solvent; or
(iii) biocompatible solvent comprising an additive that provides a solubility of the near-infrared (NIR) absorbing dye in that solvent that is higher than the solubility of the near-infrared (NIR) absorbing dye in the absence of the additive, preferably a surfactant or alkali metal salt, preferably independently present at a level in a range from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the direct treatment composition;
(iv) a biocompatible, deuterated solvent, preferably D₂O;
(v) a biocompatible solvent comprising oxygen dissolved at a level that is higher than the equilibrium concentration of oxygen when exposed to ambient atmospheric air; or
(vi) a combination of two or more of (i) to (v).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present application is further understood when read in conjunction with the appended drawings. For the purpose of illustrating the subject matter, there are shown in the drawings exemplary embodiments of the subject matter; however, the presently disclosed subject matter is not limited to the specific methods, devices, and systems disclosed. In addition, the drawings are not necessarily drawn to scale. In the drawings:

FIG. 1 illustrates a comparison of a healthy eye, a myopic eye showing scleral thinning and an eye with a staphyloma (upper) and a schematic treatment scheme for minimally invasive procedure to reinforce the sclera. The formulation is injected to the back of the eye and then a NIR light source is used from the front of the eye. NIR light penetrates further through biological tissue, allowing for delivery of light through the front of the eye to the sclera where the formulation is photoactivated.

FIG. 2 shows relative absorption of certain tissues/fluids as a function of wavelength and a preferred optical window for irradiation.

FIG. 3 shows the increases in averaged Instron measurements Young's modulus on irradiating bovine gelatin and porcine sclera in the presence of ICG and oxygen, according to Example 1.2. of the Young's Modulus for two types of materials. Bovine gelatin and porcine sclera both show a significant increase in stiffness that is reflected in the increase in the material's Young's Modulus values (26% and 21%, respectively).

FIG. 4 shows UV-Vis spectra demonstrating a decrease in ICG and DPBF signal upon irradiation at 810 nm in water with a 375 mW LED.

FIGS. 5(A-D) show the effect of solvent on singlet oxygen generation. FIG. 5(A) shows the structure of IR-820, a derivative of indocyanine green (ICG) with increased singlet oxygen generation. FIG. 5(B) and FIG. 5(C) show results of UV-Vis studies showing singlet oxygen generation of IR-820, in H₂O and D₂O, respectively. 1,3-diphenylisobenzofuran (DPBF) is consumed in the presence of singlet oxygen, causing its absorbance (˜420 nm) to decrease. The conditions where the same for both images with the only difference being the solvent. FIG. 5(D) provides a comparison of DPBF decay with the two solvents. Both H₂O and D₂O showed minimal change in DPBF decay until the 810 nm LED was turned on. A marked increase in the signal decay was observed when irradiated for both conditions. The D₂O showed a faster decay rate compared to H₂O, confirming the increased lifetimes of singlet oxygen in deuterated solvents.

FIG. 6 shows a comparison of singlet oxygen generation comparison. An increase in fluorescence indicates generation of singlet oxygen. Riboflavin was used as a desired benchmark and we measured ICG in both H₂O and D₂O. A large increase in fluorescence activation is observed when moving to the deuterated solvent.

FIGS. 7(A-B) show results of eye expansion assay—whole eye treatment and area analysis. FIG. 7(A) shows the overlay of the initial and 12-hour time point of the experiment. The middle images show area measurements for both time points and conditions to demonstrate the data used for the comparison measurements. In this example, a 20% increase in area is seen for the untreated eye and only a 2% increase in area is observed for the treated eye. This analysis focuses on scleral expansion comparisons instead of corneal expansion. FIG. 7(B) shows the compiled area analysis for the 12-hour and 24-hour time points and comparison of the treated and untreated sides. Both time points show a reduction of ˜60% in expansion between the treated and untreated portions of the eye.

FIG. 8 shows results of eye expansion assay—whole eye treatment and scleral area analysis. The treatment formulation here used sodium iodide (NaI) as the additive to aid in dissolving ICG instead of benzalkonium chloride (BAC) in D₂O. The graph shows the compiled area analysis for the 12 hour and 24 hour time points and comparison of the treated and untreated sides. Both time points show a reduction of ˜62% and ˜70%, respectively, in expansion between the treated and untreated portions of the eye.

FIG. 9 shows additional results of eye expansion assay—whole eye treatment and scleral area analysis. The ICG treatment formulation here used sodium iodide (NaI) as the additive and D₂O. The graph was a comparison of the full treatment (ICG/NaI/D₂O with NIR light) versus NIR light treatment only. The graph shows the compiled scleral area analysis for the 12 hour and 24 hour time points and comparison of the two conditions. Both time points show a reduction of ˜69% and ˜72%, respectively, in expansion between the fully treated eyes and NIR light only treated eyes. This shows that NIR light is not enough to induce expansion reduction and that ICG is necessary in the treatment.

FIGS. 10(A-B) show additional results of eye expansion assay—split-eye treatment and area analysis. FIG. 10(A) shows the area measurement of the untreated and treated portion of the eye at the initial and 24-hour time points. In this example, a 24% increase is seen for the untreated side and an 11% increase is seen for the treated side. The split eye test eliminates some biological variability by incorporating both conditions into the same eye expansion. For these experiments, an asymmetric expansion was observed overtime. FIG. 10(B) shows the compiled area analysis for the 12-hour and 24-hour time points and comparison of the treated and untreated sides. Both time points show a reduction of ˜50% in expansion between the treated and untreated portions of the eye.

FIG. 11 shows schematic representation of injection of a near-infrared-(NIR) photoactive direct treatment composition into region of posterior pole sclera.

FIG. 12 shows illustration of a representative procedure to irradiate a sclera. After adequate diffusion of photoactive direct treatment composition into the posterior pole sclera, irradiation via the pupil is performed to effect sclera crosslinking.

FIG. 13 illustrates representative structures where Y is a cationic heteroaryl moiety.

FIG. 14(A) illustrates the results of irradiation of IR-1061-pyridinium with DPBF. The structure of DPBF is shown next to its absorbance peak. FIG. 14(B) illustrates the results of irradiation of IR-1061 with DPBF.

FIG. 15(A) illustrates results of irradiation of IR-1061-acridinium with DPBF at 980 nm in CDCl₃. FIG. 15(B) illustrates results of irradiation of IR-1061-acridinium with DPBF at 980 nm in freeze-pump-thawed CDCl₃. FIG. 15(C) illustrates results of irradiation of IR-1061-acridinium at 980 nm in D₂O (with 7.5% DMSO-D6 for solubility).

FIG. 16 illustrates results of irradiation experiments with IR-1061 with DPBF in CDCl₃ at 1064 nm.

FIG. 17 illustrates results of irradiation of IR-1061-acridinium at 1064 nm in CDCl₃. Rapid bleaching of both the dye and DPBF signal was observed during irradiation.

FIG. 18 illustrates results of IR-1061-acridinium irradiation experiments at 980 nm in 15% D₆-DMSO in deuterated water.

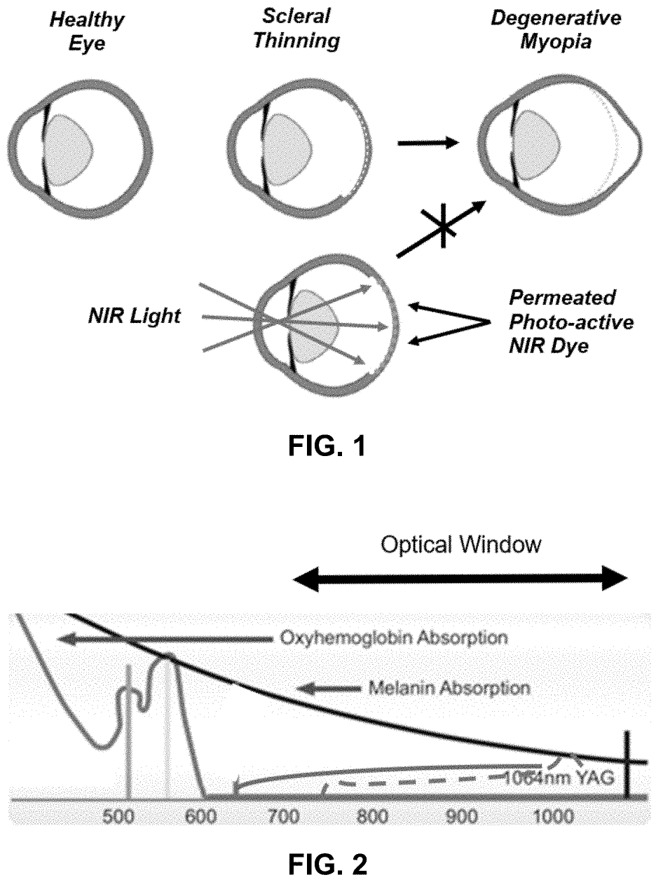

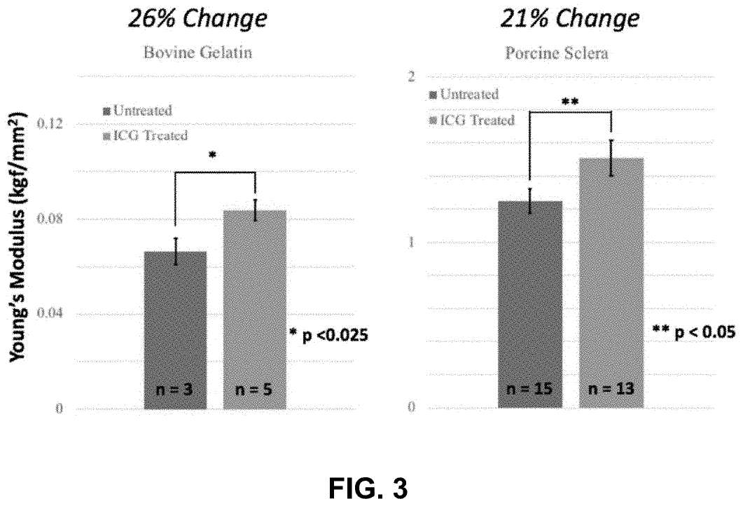

### DETAILED DESCRIPTION OF ILLUSTRATIVE EMBODIMENTS

This disclosure is directed to methods of affecting the integrity of tissue through use of singlet oxygen generated by near-infrared light, and compositions associated or useful for affecting these methods.

As set forth further below, the methods of the present disclosure comprise a step of irradiating a photoactive composition with at least one wavelength of near infrared (NIR) light.

### Methods of Treatment—Direct Treatments Using the Photoactive Compositions

Methods and compositions for treatment and/or prevention of myopia and other ocular conditions are presented herein. In some aspects, the myopia (or glaucoma) may be treated or prevented through strengthening of the sclera, reducing the stretching of the sclera, reducing staphyloma formation, increasing the modulus of the sclera, reducing the compliance of the sclera, and/or reducing the creep in the sclera, for example. In particular, the scleral tissue may be fortified, provide greater mechanical stability to the sclera, and/or prevent further reduction of the strength and/or thickness of scleral tissue by altering its chemical and/or physical structure. This can be accomplished in a number of suitable compositions and methods of use thereof in the invention. In some aspects, the methods are directed to strengthening the cornea, for example in the treatment of keratoconus. The disclosure is also directed, at least in part, to the treatment of bacterial and fungal infections, and tumors (e.g., photodynamic therapy) of the eye.

In methods of the present disclosure, involving direct treatment of the tissue, specifically altering one or more mechanical and/or chemical property of a tissue in a patient, the method comprises irradiating any one of the photoactive compositions described herein with near infrared light, under conditions specific to the generation of singlet oxygen, wherein the photoactive composition is preferably adjacent to or contacts or has permeated the tissue; wherein the irradiating results in a change in the mechanical and/or chemical property of a tissue in a patient.

In some aspects the methods comprise methods of altering a mechanical and/or chemical property of a tissue, the methods comprising irradiating a near-infrared-(NIR) photoactive direct treatment composition with near-infrared light in the presence of oxygen; wherein the near-infrared (NIR) photoactive direct treatment composition comprises a near-infrared dye that generates singlet oxygen when irradiated with near-infrared light in the presence of oxygen; and preferably wherein the near-infrared (NIR) photoactive direct treatment composition is adjacent to (contacts) or has permeated the tissue.

Additionally, or alternatively, the irradiating results in a change in the mechanical and/or chemical property of a tissue in the patient, preferably a desirable improvement of the mechanical and/or chemical property (e.g., strengthening or stiffening of the tissue).

Additionally, or alternatively, the tissue is a collagen-containing tissue. In certain aspects, the methods are operative in vivo in a patient. In other aspects, the methods are operative ex vivo.

In specific aspects of the disclosure, the methods and compositions may be used for human patients, though the methods may be useful for other mammals, such as a horse, cow, dog, cat, goat, sheep, or pig, for example.

In specific aspects, the tissue is an ocular tissue. In other specific aspects, the ocular tissue includes at least a portion of a cornea and/or a sclera. In still other specific aspects, the ocular tissue includes at least a portion of a lamina cribrosa.

Such treatments are typically provided to patients who have, or are at risk of developing, an ocular deformation condition comprising one or more of degenerative myopia, regular myopia, scleral staphyloma, keratoconus, or glaucoma.

In these embodiments, the mechanical and/or chemical property being altered by the treatment includes tensile strength, compression strength, flexural strength, modulus, elongation, or toughness of the tissue. The treatment may also result in the strengthening the tissue, stabilizing the tissue shape, changing the shape of the tissue, or a combination thereof.

The localized generation of singlet oxygen in the eye is also useful for the treatment of bacterial and fungal infections, and tumors (e.g., photodynamic therapy) in the eye.

These methods further comprise administering the photoactive composition, preferably a photoactive direct treatment composition, to the tissue of the patient, either topically (e.g., by eyedrops) or by ocular or intravenous injection. Each of these modes of administrations is considered an independent aspect of this disclosure. Where the photoactive composition is administered to the sclera, for example, such administration can be by posterior subtenon's, suprachoroidal, intravenous, or retrobulbar injection, or other suitable injection.

The time between delivery of the photoactive composition and irradiation may be adjusted for individual patients and may depend on a variety of factors, including the diffusion rate of the photoactive composition into the target tissue. The photoactive composition may be provided to the individual, and then following an amount of time to ensure that it has reached a particular location and/or sufficient level, for example, the irradiation may then be applied. For example, the photoactive composition may be monitored with slit lamps and/or confocal microscopes while the photoactive composition reaches a certain depth in a particular tissue, and then the photoactive composition is activated with light. In a particular example, the photoactive composition is monitored while it penetrates the cornea to a certain depth, and then the photoactive composition is activated with light. The amount of time between delivery and photoactivation of the photoactive composition may be of any suitable duration.

Wherein the tissue is an ocular tissue, the photoactive composition directly treats or directly reduces the risk of the ocular deformation condition. In related aspects, the tissue is an ocular tissue and a therapeutically effective amount of the photoactive composition treats a symptom of the ocular deformation condition by strengthening the ocular tissue, stabilizing the ocular tissue shape, changing the shape of the ocular tissue, or a combination thereof.

The type and duration of the irradiation by the near-infrared (NIR) light may be of any suitable kind so long as the target dye(s) are activated from the light to generate singlet oxygen. In some aspects, the light exposure is continuous, although in some cases it is intermittent or pulsed. The specific duration depends, for example, on the nature of the light source and the concentrations of the photoactive composition. Exemplary light sources for NIR light irradiation include lamps, lasers, and light-emitting diodes (LED). Light is generally used at an intensity of 10-500 mW/cm² with the particular light intensity dependent on, among other factors, the tissues and compound(s) involved. Individual aspects include those where the intensity is in a range of from 10 to 50 mW/cm², from 50 to 100 mW/cm², from 100 to 200 mW/cm², from 200 to 300 mW/cm², from 300 to 400 mW/cm², from 400 to 500 mW/cm², from 500 to 750 mW/cm², from 750 to 1000 mW/cm², or a range derived from the combination of two or more of these ranges. One of skill in the art will readily be able to adjust light intensity and time of illumination for a particular application.

Treatments may be repeated in the individual as needed. For example, a second or more treatment may be applied within days of a previous treatment, within weeks of a previous treatment, or within months of a previous treatment.

Specific aspects include, but are not limited to, the treatment of a patient having an ocular condition. In some aspects, the ocular condition comprises degenerative myopia, regular myopia, scleral staphylomas, glaucoma, normal tension glaucoma, and ocular hypertension. In other aspects, the ocular condition is or comprises keratoconus and other ectatic corneal conditions and corneal infections (infectious keratitis). In still other aspects, generated singlet oxygen is used in treating infectious organisms (bacterial and fungal), which is useful, for example, in treating corneal infections, because infrared light penetrates more deeply into cornea than visible and UV light. We also contemplate using method to treat ocular tumors, such as pigmented choroidal melanoma. In some aspects, the methods herein may be used prophylactically to reduce the risk of or prevent an ocular deformation condition including any of the foregoing. In other aspects, the treatments are designed to correct or slow the progression of one or more of these conditions in a patient where the conditions already exist.

In an exemplary procedure, following direct application of the respective photoactive composition, the eye is irradiated with NIR light for a time and under conditions sufficient to effect the desired change, the specific conditions depending on the nature of the treatment and specific composition of the irradiated material. Suitable modes of clinical implementation of irradiation include having the patient in a supine position and delivering light through an operating microscope or having the patient seated and delivering light using a slit lamp system, an indirect ophthalmoscope, or other suitable light source. Because NIR light is used, the light may be delivered through the patient's pupil or other portion of the eye.

In independent aspects, the directly applied photoactive composition may be irradiated entirely or in targeted areas. In separate aspects, individual portions of the directly applied photoactive compositions may be irradiated separately, either positionally or temporally, or both. Irradiation may involve a patterned application of light. Suitable exemplary methods to control the irradiation pattern incident on the tissue include rastering the irradiation beam, using a spatial light modulator, using a digital mirror device, or using a fiber optic coupled to a laser. The amount of light exposure may also be changed to adjust the degree of polymerization or crosslinking that is occurring in the tissue. The exposure of the NIR light may directed to a particular region of the sclera, as identified by diagnostic imaging. Exemplary diagnostic imaging techniques include ultrasound imaging, optical coherence tomography (OCT) imaging, OCT Doppler imaging, or magnetic resonance imaging (MRI).

Additionally, in separate aspects, these methods further comprise determining the type of treatment that is required or desired prior to treatment.

Further, any of these processes may be repeated, after waiting a suitable time to evaluate effect of the change of the properties.

Operable Dyes

In the described methods, the near-infrared-(NIR) photoactive direct treatment composition comprises a near-infrared (NIR) absorbing dye having a heptamethine linkage.

But more generally the near-infrared (NIR) absorbing dye expected to be useful in these methods include those comprising a cyanine structure, a pyrylium structure, or a thiopyrylium structure, or a combination thereof.

Cyanine dyes consist of 2 nitrogen heterocycles connected by a conjugated carbon chain. Generally, for the dyes considered herein, the chain contributes to the overall shape of the molecule being linear, and the length of the chain determines the wavelength at which the dyes absorb and fluoresce light. In the context of the present disclosure, cyanine dyes include, but are not limited to those described in U.S. Pat. Nos. 4,464,383; 5,563,028; 5,633,390; 5,973,158; 6,072,059; 6,515,811; 6,673,943; 9,610,370; and 10,280,307; each of which are incorporated by reference herein at least for its descriptions of the near-infrared (NIR) absorbing dye portion of the claimed compounds (including backbones and substitution patterns) and for its teachings of the methods of making and using the same.

Additionally, or alternatively, in certain aspects, the methods include the use of compositions where the near-infrared (NIR) absorbing dye comprises a structure of:

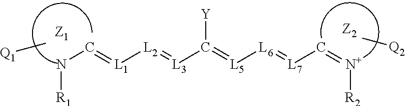

wherein

L₁, L₂, L₃, L₅, L₆, and L₇ are substituted or unsubstituted methines, wherein the optional substituents are independently C₁₋₆ alkyl or C₂₋₆ alkenyl; or L₁ and L₃, or L₃ and L₅, or L₅ and L₇ may be linked with a C₂₋₄ alkylene or C₂₋₄ alkenylene substituent to form a 5- to 7-membered ring;

each of Z¹ and Z² is independently a five- or six-membered nitrogen-containing heterocyclic ring, optionally fused to another aryl or heteroaryl ring;

each of Q₁ and Q₂ is independently H or a substituent positioned on the five- or six-membered nitrogen-containing heterocyclic ring and/or the optionally fused aryl or heteroaryl ring, each optional substituent comprising an optionally substituted C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof, —(C₀₋₁₂alkyl)borate,

R₁ and R₂ is independently C₁₋₁₂ alkyl, —[CH₂—CH₂—O—]₁₋₆R¹⁰, —(C₀₋₁₂alkyl)amino acid residue, or a 5- or 6-member ringed aryl or heteroaryl, each of which may be optionally substituted with one or more —(C₀₋₁₂alkyl)(SO₃)—R¹⁰ or a salt thereof, —(C₀₋₁₂alkyl)(SO₄)—R¹⁰ or a salt thereof, —(C₀₋₁₂alkyl)(PO₄)—R¹⁰ or a salt thereof, —(C₀₋₁₂alkyl)OR¹⁰, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof or —(C₀₋₁₂alkyl)borate or borate ester;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substitutedaryloxy, optionally substituted heteroaryloxy, halogen, or optionally substituted cationic heteroaryl moiety;

or a geometric, conformational, or rotational isomer thereof.

In certain aspects, these dyes include their salt forms.

Additionally, or alternatively, within this context, while Q₁, Q₂, R₁, and R₂ are defined in terms of specific optional substituents, and Y is defined merely as “optionally substituted,” the optional substituents may include not only those specific substituents, but may also include those independent substituents defined elsewhere herein as “Fn.”

Also additionally, or alternatively, within this context, while Y is defined in these dyes as present in the L₄ position (i.e., between the L₃ and L₅ methines), and is preferably positioned there, in other aspects, Y can be alternatively positioned on any of the L₁, L₂, L₃, L₄, L₅, L₆, or L₇ positions. Preferably, Y is bonded directly to the heptamethine linkage (i.e., no intermediary linking groups).

Additionally, or alternatively, Z₁ and Z₂ may be the same or different.

Additionally, or alternatively, the five- or six-membered nitrogen-containing heterocyclic ring of Z₁ and Z₂ can independently comprise a pyrrole ring, imidazole ring, isothiazole ring, isoxazole ring, oxadiazole ring, oxazole ring, pyrazole ring, pyrimidyl, thiazole ring, selenazole ring, thiadiazole ring, triazole ring, or a pyridine ring.

Additionally, or alternatively, the five- or six-membered nitrogen-containing heterocyclic ring of Z₁ and Z₂ is independently fused to a phenyl, naphthyl, pyridinyl, quinolinyl, quinoxalinyl, N-alkyl-benzoindolenine, dibenzofuranyl, or dibenzothiophenyl.

Additionally, or alternatively, Z₁ and Z₂ independently comprise a benzimidazole ring, benzindole ring, benzoindolenine ring, benzoxazole ring, benzothiazole ring, furopyrrole ring, imidazole ring, imidazoquinoxaline ring, indolenine ring, indolizine ring, isoxazole ring, naphthimidazole ring, naphthothiazole ring, naphthoxazole ring, oxazolocarbazole ring, oxazole ring, oxazolodibenzofuran ring, pyrrolopyridine ring, pyridine ring, quinoline ring, quinoxaline ring, thiazole ring, or naphthoimidazole ring.

In specific aspects, the methines not bonded to Y are otherwise not substituted.

In other specific aspects, one of L₁ and L₃, or L₂ and L₄, or L₃ and L₅, or L₄ and L₆, or L₅ and L₇ are linked with a C₂₋₄ alkylene substituent to form a 5- to 7-membered ring. In one exemplary example within this context, the near-infrared (NIR) absorbing dye comprises a structure of:

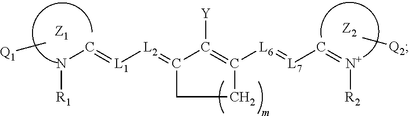

where L₁, L₂, L₃, L₄, L₅, L₆, L₇, Q₁, R₁, Y, Z₁, are defined in any of the definitions as set forth elsewhere herein, for these features, in any combination or permutations and m is 1, 2, or 3.

In other specific aspects the near-infrared (NIR) absorbing dye comprises a structure of:

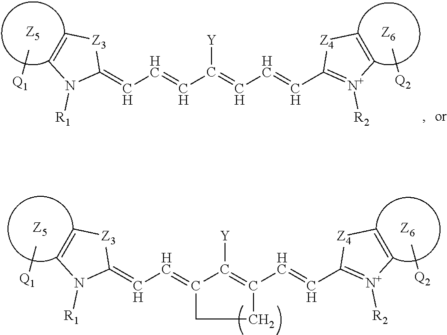

wherein

each of Z₃ and Z₄ is independently —CR¹¹R¹², —NR¹¹, —O—, —S— or —Se— (each of Z₃ and Z₄ is independently preferably —CR¹¹R¹², —NR¹¹, —O— or —S—, each of Z₃ and Z₄ is independently more preferably is —CR¹¹R¹², —O— or —S, each of Z₃ and Z₄ is independently further preferably is —CR¹¹R¹² or —O—, and each of Z₃ and Z₄ is independently most preferably —CR¹¹R¹²);

each of Z₅ and Z₆ is independently preferably phenyl, naphthyl, pyridinyl, quinolinyl, quinoxalinyl, N-alkyl-benzoindolenine, dibenzofuranyl, or dibenzothiophenyl;

each of R¹ and R² are defined in any of the definitions as set forth elsewhere herein;

each of R¹¹ and R¹² is independently a C₁₋₆ alkyl, preferably methyl; and

Q₁ and Q₂ are independently, preferably H, —COOH or a salt thereof, or —SO₃H or a salt thereof.

In still other specific aspects the near-infrared (NIR) absorbing dye comprises a structure of:

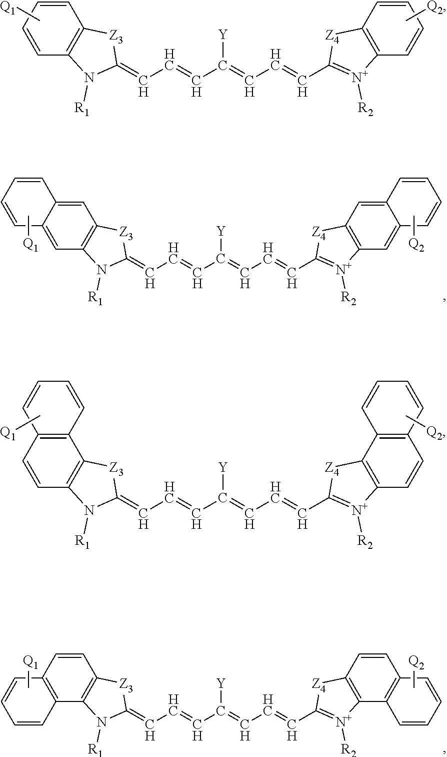

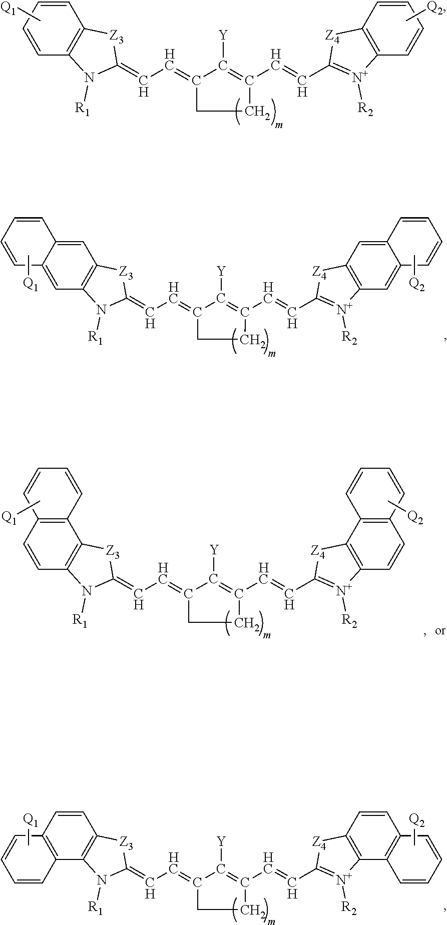

wherein each of Z₃ and Z₄ is independently —CR¹¹R₁₂, —NR¹¹, —O—, —S— or —Se— (each of Z₃ and Z₄ is independently preferably —CR¹¹R₁₂, —NR¹¹, —O— or —S—, each of Z₃ and Z₄ is independently more preferably is —CR¹¹R¹², —O— or —S, each of Z₃ and Z₄ is independently further preferably is —CR¹¹R¹² or —O—, and each of Z₃ and Z₄ is independently most preferably —CR¹¹R¹²);

each of R¹¹ and R¹² is independently a C₁₋₆ alkyl, preferably methyl;

m=1, 2, or 3; and

Q₁ and Q₂ are independently, preferably H, —COOH or a salt thereof, or —SO₃H or rotational or conformational isomer or a salt form thereof.

Additionally, or alternatively, within the context of the immediately preceding structures, the fused naphthalene moiety may be replaced with an optionally substituted quinolinyl, quinoxalinyl, N-alkyl-benzoindolenine, dibenzofuranyl, or dibenzothiophenyl ring, and these provide additional aspects of of this disclosure.

In still other specific aspects the near-infrared (NIR) absorbing dye comprises a structure of:

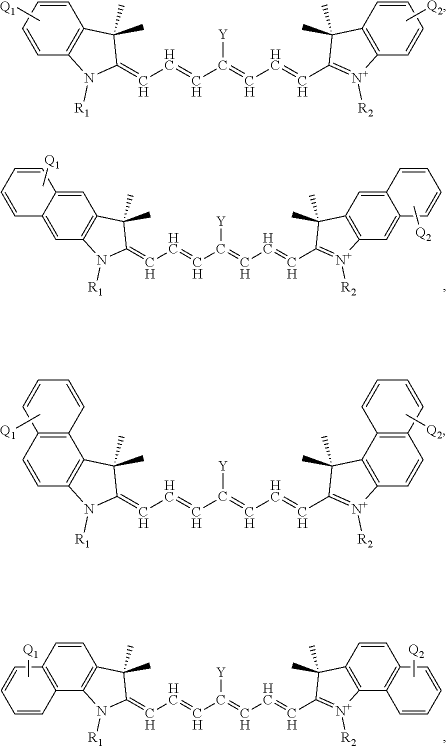

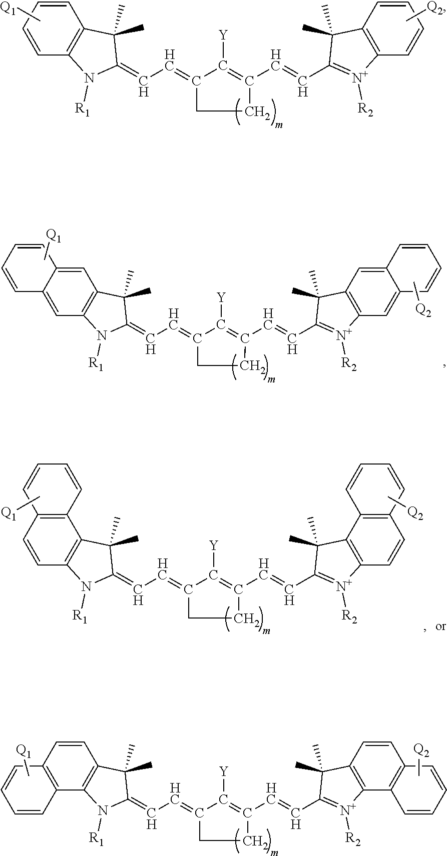

where R₁ and R₂ are independently —(C₁₋₁₂alkyl)(SO₃)H or a salt thereof or —(C₁₋₁₂alkyl)COOH; or a rotational or conformational isomer or a salt form thereof.

In certain independent Aspects of this Embodiment, the fused naphthalene moiety may be replaced with an optionally substituted quinolinyl, quinoxalinyl, N-alkyl-benzoindolenine, dibenzofuranyl, or dibenzothiophenyl ring.

In even more specific aspects, the near-infrared (NIR) absorbing dye comprises a structure:

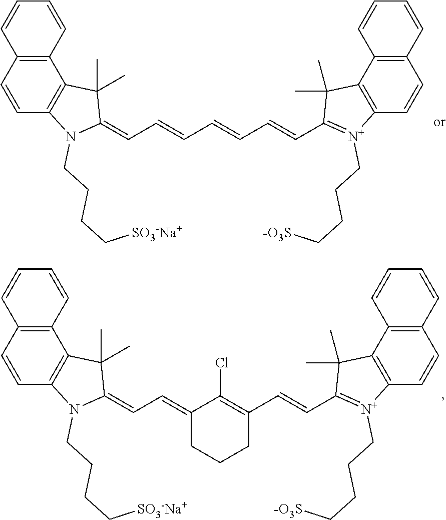

or a rotational or conformational isomer or alternative salt form thereof. For the sake of clarity, these two structures may be considered a rotational or conformational of one another:

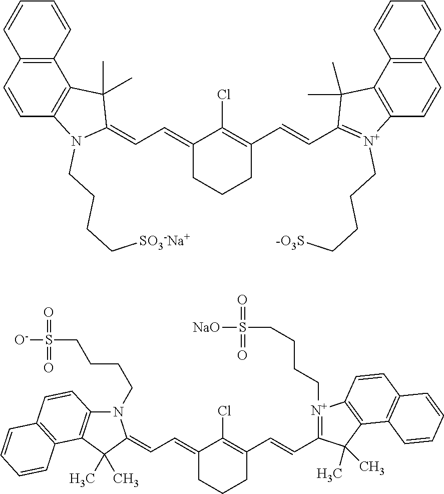

The disclosed methods may also employ the use of pyrylium dye or thiopyrylium dyes. In certain aspects, the pyrylium dye or the thiopyrylium dye includes dyes that are described in U.S. Pat. No. 4,283,475 that are incorporated by reference for its teachings of these types of dyes, and the ability to functionalize and make these dyes.

Additionally, or alternatively, within the context of pyrylium or thiopyrylium, the near-infrared (NIR) absorbing dye, in some aspects, comprises a structure of:

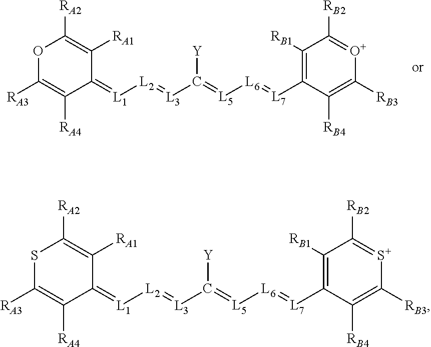

respectively, wherein

L₁, L₂, L₃, L₅, L₆, and L₇ are substituted or unsubstituted methines, wherein the optional substitutents are independently C₁₋₆ alkyl or C₂₋₆ alkenyl; or L₁ and L₃, or L₃ and L₅, or L₅ and L₇ may be linked with C₂₋₄ alkylene or C₂₋₄ alkenylene substituents;

R_{A1}, R_{A2}, R_{A3}, R_{A4}, R_{B1}, R_{B2}, R_{B3}, and R_{B4} are each independently H, deutrium, or tritium, an C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂ or a salt thereof, —(C₀₋₁₂alkyl)N(R¹⁰)₂ or a salt thereof, —(C₀₋₁₂alkyl)borate;

n is independently 0, 1, 2, 3, or 4, preferably 2;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substituted aryloxy, optionally substituted heteroaryloxy, halogen, or optionally substituted cationic nitrogen-containing heteroaryl moiety,

or a rotational or conformational isomer or a salt form thereof.

Likewise, as set forth above for the cyanine dyes, it should be appreciated that while Y is defined merely as “optionally substituted,” the optional substituents may also include those defined elsewhere herein as “Fn.” Also, in additional to the specifically defined descriptions of R_{A1}, R_{A2}, R_{A3}, R_{A4}, R_{B1}, R_{B2}, R_{B3}, and R_{B4}, these substituents may also independently be any one or more of these Fn substituents.

Also additionally, or alternatively, within this context, while Y is defined in these dyes as present in the L₄ position (i.e., between the L₃ and L₅ methines), and is preferably positioned there, in other aspects, Y can be alternatively positioned on any of the L₁, L₂, L₃, L₄, L₅, L₆, or L₇ positions. Preferably, Y is bonded directly to the heptamethine linkage (i.e., no intermediary linking groups).

Additionally, or alternatively, Z₁ and Z₂ may be the same or different.

In specific aspects, the methines not bonded to Y are otherwise not substituted.

In other specific aspects, one of L₁ and L₃, or L₂ and L₄, or L₃ and L₅, or L₄ and L₆, or L₅ and L₇ are linked with a C₂₋₄ alkylene substituent to form a 5- to 7-membered ring. In one exemplary example within this context, the near-infrared (NIR) absorbing dye comprises a structure of:

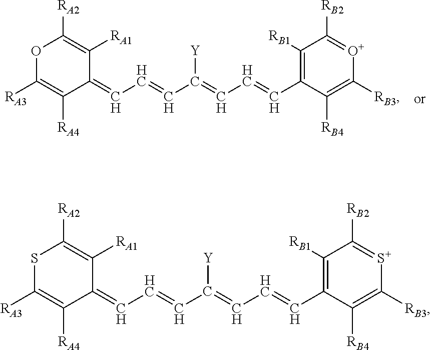

or a rotational or conformational isomer or a salt form thereof.

In certain aspects, R_{A1}, R_{A4}, R_{B1}, and R_{B4} are H, or an isotope thereof, and R_{A2}, R_{A3}, R_{B2}, and R_{B3} are aryl, heteroaryl, or branched alkyl preferably phenyl, pyridinyl, or tert-butyl.

In the cyanine, pyrylium or thiopyrylium dyes discussed above, Y has been defined in terms of H, or an optionally substituted amine, optionally substituted alkyl, optionally substituted alkoxy, optionally substituted aryl, optionally substituted heteroaryl, optionally substituted aryloxy, optionally substituted heteroaryloxy, or halogen, or an optionally substituted cationic heteroaryl ring moiety. Each of these definitions of Y represents an independent aspect of this disclosure. In those cases where Y is been defined in terms of H, or an optionally substituted amine, optionally substituted alkyl, optionally substituted alkoxy, optionally substituted aryl, optionally substituted heteroaryl, optionally substituted aryloxy, optionally substituted heteroaryloxy, or halogen, the methods of making these dyes can be identified by those skilled in the art without undue burden, using at least the references cited elsewhere herein for this purpose.

However, in the case where Y is an optionally substituted cationic heteroaryl ring moiety such methods are not believed to be known, except by methods set forth in a co-filed, co-pending application, client reference number 103693.000491/CIT-8117, titled “NEAR-INFRARED HEPTAMETHINE DYES FOR GENERATION OF SINGLET OXYGEN,” which is incorporated by reference herein in its entirety for all purposes, or at least for its teaching of the methods of making and using, the compositions themselves, and their activities in generating singlet oxygen.

In certain aspects, then, where Y is an optionally substituted cationic heteroaryl ring moiety, Y may also be independently defined as an optionally substituted cationic nitrogen-, oxygen, or sulfur-containing heteroaryl ring moiety. In the context of the cationic heteroaryl ring moiety, the cationic charge is distributed as a formal charge within the ring structure of the heteroaryl ring moiety, as opposed to residing on one or more of the optional substituents.

In certain aspects, the optionally substituted cationic nitrogen-containing heteroaryl ring is bonded to the heptamethine linkage by a C—C bond or a C—N bond. In certain Aspects of this Embodiment, the optionally substituted cationic oxygen- or sulfur-containing heteroaryl ring is bonded to the heptamethine linkage by a C—C bond.

In other aspects, the optionally substituted cationic heteroaryl ring moiety is orthogonally coupled to the heptamethine linkage. In some aspects, the optionally substituted cationic heteroaryl ring moiety is characterized as a charge-transfer partner of the near-infrared (NIR) absorbing dye.

In other aspects, the optionally substituted cationic heteroaryl ring moiety comprises an optionally substituted acridinium, benzoxazolium, benzothiazolium, imidazolium, isoxazolium, isoquinolinium, isothiazolium, naphthoimidazolium, naphthothiazolium, naphthoxazolium, oxazolium, pyrazinium, pyrazolium, pyridimium, pyridinium, quinolinium, tetrazinium, tetrazolium, thiazolium, triazinium, triazolium, benzopyrazinium, benzopyridimium, benzopyridinium, naphthopyrazinium, naphthopyridimium, benzopyridinium, benzotriazinium, naphthotriazinium moiety, pyrylium, chromenylium, xanthylium moiety, thiopyrylium, thiochromenylium, or thioxanthylium moiety. The optional substituents may comprise any one or more of the functional group “Fn” a set forth elsewhere herein.

Embodiment 30. The method of Embodiment 12 to 29, wherein the optionally substituted cationic heteroaryl moiety comprises an optionally substituted structure of:

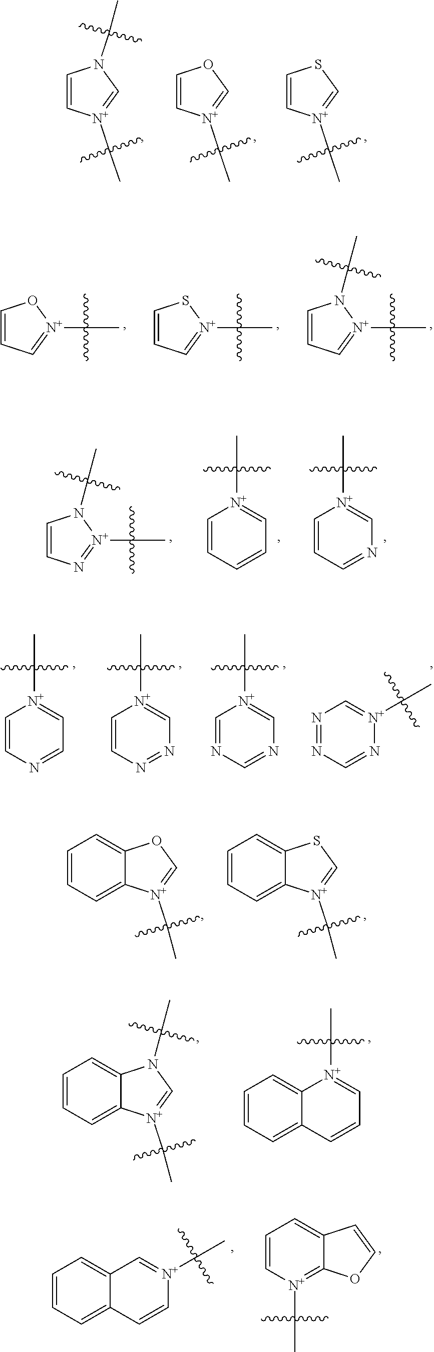

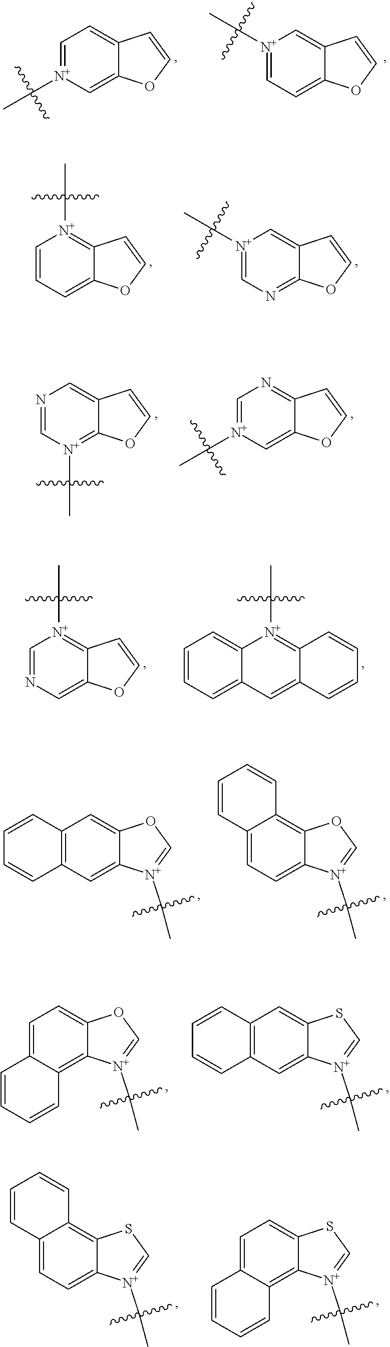

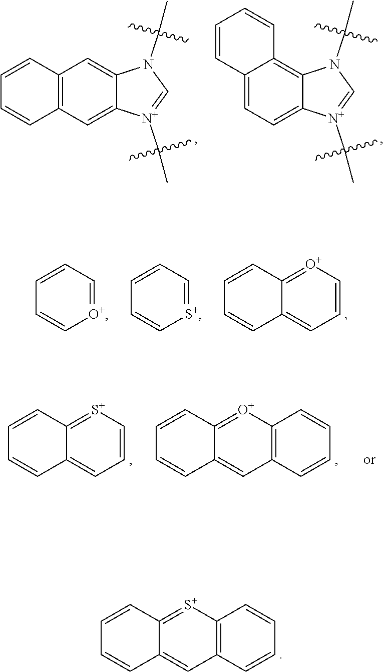

Throughout this disclosure, the structures have been defined in terms of an optional salt form. This accounts for the fact that, the structures generally comprise at least one cationic group (i.e., carry a positive charge), but they also may also contain substituents comprising anionic groups (i.e., carry a negative charge). Depending on the number and nature of these charged substituents, the structures may carry a net positive or negative charge or are net charge neutral. In some cases, a net neutral charge may arise from the amphoteric nature of the compound (i.e., internally net charge balanced). Alternatively, or additionally, the cationic groups may have associated counter anions and the anionic groups may have associated counter cations. In either case, the counter ions need not be seem as particularly limiting, but in preferred aspects, the counter anions are halide anions (e.g., fluoride, chloride, bromide, and/or iodide), or other inorganic anions (e.g., perchlorate, tetrafluoroborate, hexafluorophosphate, sulfate, hydrogensulfate and/or nitrate) or organic anions (e.g., such as trifluoroacetate, trichloroacetate, triflate, mesylate, and/or p-toluenesulfonate ions). Preferred counter cations include ammonium or alkali metal cations, such as Li⁺, Na⁺, or K⁺.

In other aspects, the near-infrared (NIR) absorbing dye may be substituted with, or is conjugated to at least one isotope of carbon, chlorine, fluorine, hydrogen, iodine, nitrogen, or oxygen enriched above its natural abundance. In some aspects, the isotope is a radioisotope.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

As the methods rely on near-infrared (NIR) dyes for generating singlet oxygen, the irradiating is done with a near-infrared light having a wavelength in a range of from 750 nm to 800 nm, from 800 nm to 850 nm, from 850 nm to 900 nm, from 900 nm to 950 nm, from 950 nm to 1000 nm, from 1000 nm to 1050 nm, from 1050 nm to 1100 nm, from 1100 nm to 1150 nm, from 1150 nm to 1200 nm, from 1200 nm to 1250 nm, from 1250 to 1300 nm, from 1300 to 1350 nm, from 1350 nm to 1400 nm, or in a range comprising two of more of these foregoing ranges. At these wavelengths, the dyes typically exhibit a local λₘₐₓ for light absorption and once irradiated, fluoresce and/or generate single oxygen when the irradiation is done in the presence of oxygen.

As exemplified herein and in the co-pending application set forth elsewhere, certain additional materials and or conditions have been showed to amplify the generation of singlet oxygen. These additional materials and or conditions are discussed further elsewhere. But typically, the near-infrared (NIR) photoactive direct treatment composition, in addition to the near-infrared dye, comprise or is associated with a solvent, preferably biocompatible, which allows for the inclusion of these additional materials, and favor the preferred conditions. For example, the near-infrared (NIR) photoactive direct treatment compositions may comprise solvents that enhance the solubility of oxygen, additives that enhance the solubility of the dye in the direct treatment composition, and deuterated solvents or compounds that improve the lifetime of the singlet oxygen once generated. Further, the deliberate addition of oxygen to the direct treatment compositions, beyond that available from simple equilibration of the compositions with ambient atmospheric air, provides for enhanced levels of singlet oxygen generation. In certain aspects, the effects of these materials and conditions are additive, favoring the inclusion of two or more, preferably three or more in the composition.

In certain aspects, then, the near infrared dye is dissolved or suspended or is otherwise associated with in the solvent, preferably a solvent that is biocompatible with and/or physiologically acceptable to the patients, preferably human patients and their associated tissues and biological systems. It is clearly advantageous that the solvent be optically transparent in the UV-VIS and near-infrared range of the optical spectrum. Aqueous solvents are preferred, where aqueous is defined as comprising water (as H₂O, DOH, or D₂O). Compositions comprising D₂O are especially preferred.

In certain aspects, the direct treatment composition comprises or is associated with a solvent or solvent additive that enhances the solubility of oxygen. In certain aspects, this a solvent or solvent additive is used in combination with, or instead of, a biocompatible aqueous solvent as defined heretofore. For example, in some aspects, the solvent or solvent is or comprises a fluorinated or perfluorinated solvent. Low molecular weight fluorinated polymers or surfactants would appear to be particularly attractive in this regard. Examples of such solvents are set forth elsewhere herein.

In certain other aspects, the direct treatment composition comprises one or more physiologically acceptable additives that enhances the solubility of the near-infrared dye in the selected biocompatible solvent. In certain aspects, certain surfactants and salts are useful in this regard. Depending on the nature of the particular near-infrared dye chosen, cationic, anionic, or charge-neutral (including amphoteric) surfactants may be useful. As exemplified herein, the use of cationic surfactants, for example comprising an ammonium group such as benzalkonium salts, preferably benzalkonium chloride work well in this capacity.

In certain other aspects, the use of salts and buffers (e.g., Dulbecco's & PBS) to increase the ionic strength of the direct treatment compositions also appear to enhance the solubility of the near-infrared dyes in the compositions. Salts such as ammonium or alkali metal acetates, citrates, halides, nitrates, phosphates, sulfates, or mixtures thereof are expected to enhance the performance of the dyes in the methods. Sodium or potassium halides, especially sodium iodide, are preferred in this capacity.

In certain aspects, the surfactant or salt may be independently present in the compositions at levels ranging from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the composition.

In certain aspects, the compositions comprise or are associated with biocompatible and/or physiologically acceptable solvents that comprise deuterated solvent. As used herein, the term “deuterated solvent” refers to a solvent in which the proportion of the ordinary isotope of hydrogen in the solvent has been replaced with deuterium. In some aspects, the deuterium content of the deuterated solvent, or the entire biocompatible and/or physiologically acceptable solvent is at least twice that of its natural abundance. In some aspects, at least 5 atom %, 10 atom %, 20 atom %, 30 atom %, 40 atom %, 50 atom %, 60 atom %, 70 atom %, 80 atom %, 90 atom %, 95 atom %, 98 atom %, or 99 atom % of the hydrogen in the deuterated solvent has been replaced by deuterium. The presence of the deuterated solvent, or deuterium in the solvent appears to stabilize the singlet oxygen once formed, extending its lifetime for further reaction. In certain aspects, the deuterated solvent is or comprises deuterated dimethyl sulfoxide, deuterated methanol, deuterated ethanol, deuterated tetrahydrofuran, or deuterated water.

In still further aspects, the near-infrared-(NIR) photoactive direct treatment composition is oxygenated before or during the irradiation, preferably through use of gases inert gases enriched in oxygen, for example containing, at least 30 vol %, 40 vol %, 50 vol %, 60 vol %, 70 vol %, 80 vol %, 90 vol %, or 95 vol % or pure oxygen. In preferred aspects, the direct treatment composition comprises oxygen dissolved at a level that exceeds that of the concentration of dissolved oxygen when in the presence of ambient atmospheric air. Additionally, or alternatively, the direct treatment composition comprises dissolved oxygen at a level within 50%, 45%, 40%, 35%, 30%, 25%, 20%, 15%, 10%, or 5% of the saturation limit of oxygen in the composition, when the composition is in the presence of pure oxygen.

In addition to the foregoing descriptions, the disclosure embraces not only methods of treatment, but also the compositions for use in those treatments.

In certain aspects, then, the disclosure includes those composition comprising a near-infrared dye that generates singlet oxygen when irradiated with near-infrared light in the presence of oxygen for use in any one of the foregoing methods for treating tissue. That is, in certain aspects, the disclosure sets forth near-infrared (NIR) photoactive direct treatment compositions or the near-infrared dyes that include any one or more of the features attributed to them in the foregoing descriptions of the methods.

Within these descriptions, in certain aspects, the compositions comprise

(a) compounds comprising at least one near-infrared (NIR) absorbing dye that generates singlet oxygen, when irradiated with near-infrared light in the presence of oxygen; dissolved or suspended or otherwise associated with

(b) one or more of
(i) an optically transparent, biocompatible solvent
(ii) a biocompatible solvent having an oxygen solubility that is greater than the oxygen solubility in H₂O under comparable oxygen partial pressures, preferably a fluorinated or perfluorinated solvent; or
(iii) a biocompatible solvent comprising an additive that provides a solubility of the near-infrared (NIR) absorbing dye in that solvent that is higher than the solubility of the near-infrared (NIR) absorbing dye in the absence of the additive, preferably a surfactant or alkali metal salt, preferably independently present at a level in a range from 100 ppm to 50 wt %, or any subrange otherwise defined herein for these additives range defined by two or more of the foregoing ranges, relative to the total weight of the direct treatment composition;
(iv) a biocompatible, deuterated solvent, preferably D₂O;
(v) a biocompatible solvent comprising oxygen dissolved at a level that is higher than the equilibrium concentration of oxygen when exposed to ambient atmospheric air;

or
(vi) a combination of two or more of (i) to (v).

Additionally, these compositions may comprise an additional crosslinking compound defined as a single crosslinking molecule or as a chain of crosslinking molecules, such as a protein, polysaccharide, carbohydrate, glycosaminoglycan, proteoglycan, or combination thereof that is native to a sclera. In specific aspects, the protein is or comprises collagen and/or glyceraldehyde.

Terms

The present invention may be understood more readily by reference to the entire description taken in connection with the accompanying Figures and Examples, all of which form a part of this disclosure. It is to be understood that this disclosure is not limited to the specific products, methods, conditions or parameters described or shown herein, and that the terminology used herein is for the purpose of describing particular aspects or embodiments by way of example only and is not intended to be limiting of any claimed invention. Similarly, unless specifically otherwise stated, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer to methods of treating tissue and patient conditions using photoactive compositions and the photoactive compositions associated with these methods. It is to be understood that, where the disclosure describes or claims a feature or aspect associated with a composition or a method of making or using a composition, that such a description or claim is intended to extend these features or aspects to each of these contexts (i.e., the description of a compound or composition also refers to that feature or aspect in the methods employing (making or using) these the compound or composition, and a description of a method employing compound or composition also refers to the feature or aspect of the compound or composition, as if separately and/or individually recited).

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

It is to be appreciated that certain features of the invention which are, for clarity, described herein in the context of separate embodiments or aspects, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The transitional terms “comprising,” “consisting essentially of” and “consisting” are intended to connote their generally in accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially” of limits the scope of a claim to the specified materials or steps “and those that do not materially affect the basic and novel characteristic(s)” of the claimed invention. In cases here, the basic and novel characteristic(s) of the compositions here are the ability to generate singlet oxygen when irradiated with near-infrared radiation in the presence of oxygen. Embodiments or aspects described in terms of the phrase “comprising” (or its equivalents), also provide, as embodiments, those which are independently described in terms of “consisting of” and “consisting essentially of.”

When a list is presented, unless stated otherwise, it is to be understood that each individual element of that list, and every combination of that list, is a separate embodiment. For example, a list of embodiments or aspects presented as “A, B, or C” is to be interpreted as including the embodiments, “A,” “B,” “C,” “A or B,” “A or C,” “B or C,” or “A, B, or C.” Similarly, a designation such as C₁₋₃ includes C₁, C₂, C₃, C₁₋₂, C₂₋₃, C_{1,3}, as separate embodiments, as well as C₁₋₃.

Throughout this specification, words are to be afforded their normal meaning, as would be understood by those skilled in the relevant art. However, so as to avoid misunderstanding, the meanings of certain terms will be specifically defined or clarified.

The term “alkyl” as used herein refers to a linear, branched, or cyclic saturated hydrocarbon group typically although not necessarily containing 1 to about 24 carbon atoms, preferably 1 to about 12 carbon atoms, such as methyl, ethyl, n-propyl, isopropyl, n-butyl, isobutyl, tent-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. The term “substituted alkyl” refers to alkyl groups substituted with one or more substituent groups, and the terms “heteroatom-containing alkyl” and “heteroalkyl” refer to alkyl groups in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl groups, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl groups substituted with one or more substituent groups, and the terms “heteroatom-containing alkenyl” and “heteroalkenyl” refer to alkenyl groups in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl groups, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to an alkynyl group substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include a linear, branched, unsubstituted, substituted, and/or heteroatom-containing alkynyl and lower alkynyl group, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aromatic” refers to the ring moieties which satisfy the Hückel 4n+2 rule for aromaticity, and includes both aryl (i.e., carbocyclic) and heteroaryl (also called heteroaromatic) structures, including aryl, aralkyl, alkaryl, heteroaryl, heteroaralkyl, or alk-heteroaryl moieties, or pre-polymeric (e.g., monomeric, dimeric), oligomeric or polymeric analogs thereof. While the descriptions of the methods and systems involving KOH are provided in terms of heteroaromatic substrates, where their operability is preferred, it is reasonably believed that they also work on aryl substrates.

The term “aryl” as used herein, and unless otherwise specified, refers to an aromatic substituent or structure containing a single aromatic ring or multiple aromatic rings that are fused together, directly linked, or indirectly linked (such that the different aromatic rings are bound to a common group such as a methylene or ethylene moiety). Unless otherwise modified, the term “aryl” refers to carbocyclic structures. Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail elsewhere herein.

The term “aryloxy” as used herein refers to an aryl group bound through a single, terminal ether linkage, wherein “aryl” is as defined above. An “aryloxy” group may be represented as -0-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2, 7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The terms “crosslink” or “crosslinking” carry their normal meaning in its broadest sense, as readily used by a person of skill in the polymer or biochemical arts. It typically refers to formation of a covalent or other bond (e.g., hydrogen bond) between two molecules, typically between two oligomers, macromers, or polymers. For example, a collagen molecule may be crosslinked to other collagen molecules to form a network of interlinked collagen molecules held together by covalent linkages.

The terms “cyclic” and “ring” refer to alicyclic or aromatic groups that may or may not be substituted and/or heteroatom-containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic. The term “acyclic” refers to a structure in which the double bond is not contained within a ring structure.

The terms “direct treatment ” and “directly treating and the like refer to the therapies described herein where a photoactive composition, preferably a photoactive direct treatment composition, directly interacts with tissue components to cause a change in the properties of that tissue. Direct treatment with a photoactive composition is distinguished from indirect treatment wherein a photoactive composition interacts with one or more other components of the contacted tissue directly to cause a change in the property of that tissue, for example, directly acting upon a sclera to crosslink the compounds of the sclera, so as to change or alter the properties of that tissue. The terms “direct treatment,” “directly treating,” “directly reducing the risk of” and the like as used herein additionally refer to the amelioration of at least one symptom of a disease or condition such as an ocular deformation condition. For example, scleral stretching, scleral thinning, or scleral weakening are symptoms of myopia. A skilled artisan recognizes that the treatment does not need to improve vision, such as improving it to its fullest extent. In particular aspects, the terms refer to preventing the progression or slowing the progression of an ocular deformation condition such as degenerative myopia or keratoconous. In a specific embodiment, the vision stabilizes.

The terms “halo,” “halide,” and “halogen” are used in the conventional sense to refer to a chloro, bromo, fluoro, or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated, and unsaturated species, such as alkyl groups, alkenyl groups, aryl groups, and the like. The term “lower hydrocarbyl” intends a hydrocarbyl group of 1 to 6 carbon atoms, preferably 1 to 4 carbon atoms, and the term “hydrocarbylene” intends a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” intends a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Non-limiting heteroaryl moieties include those an optionally substituted furan, pyrrole, thiophene, pyrazole, imidazole, triazole, isoxazole, oxazole, thiazole, isothiazole, oxadiazole, pyridine, pyridazine, pyrimidine, pyrazine, triazone, benzofuran, benzopyrrole, benzothiophene, isobenzofuran, isobenzopyrrole, isobenzothiophene, indole, isoindole, indolizine, indazole, azaindole, benzisoxazole, benzoxazole, quinoline, isoquinoline, cinnoline, quinazoline, naphthyridine, 2,3-dihydrobenzofuran, 2,3-dihydrobenzopyrrole, 2,3-dihydrobenzothiophene, dibenzofuran, xanthene, dibenzopyrol, dibenzothiophene. In more preferred embodiments or aspects, the substrate comprises a moiety comprising an optionally substituted furan, pyrrole, thiophene, pyrazole, imidazole, benzofuran, benzopyrrole, benzothiophene, indole, azaindole dibenzofuran, xanthene, dibenzopyrrole, or dibenzothiophene moiety.

Non-limiting examples of nitrogen-containin heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “mechanical and/or chemical property of a tissue” as used herein refers to a biophysical property of the tissue. Examples of a mechanical property include but are not limited to tensile strength, compression strength, flexural strength, modulus, elongation and toughness (stress-strain). These latter terms confer their normally understood meanings. Examples of a chemical property include but are not limited to the nature of chemical bonds of the tissue components (e.g. collagen versus crosslinked collagen), amount of water of hydration of the tissue is capable of retaining, the biodegradation or turnover rate of tissue constituents.

The term “mechanical stability” as used herein refers to the ability of a tissue or organ to maintain its functional shape even under the influence of stresses imposed on it.

As used herein, the term “moiety” refers to a part of a molecule which is typically given a name as it can be found within other kinds of molecules as well. In some instances, moieties may be composed of yet smaller moieties and functional groups. For example, a

As used here, “myopia,” which may also be referred to as near-sightedness, refers to the ability to clearly see objects up close but not those at a distance. The presently disclosed methods and materials are suitable for addressing all forms and degrees of myopia. In specific embodiments or aspects, myopia is pathologic and is diagnosed when eyeball elongation is associated with thinning of ocular tissues in the posterior portion of the globe. High myopia is defined as greater than 8 diopters.

The term “prevention of myopia” as used herein, and described in certain aspects, refers to the avoidance of the development or progression of myopia. Although in specific aspects the myopia is avoided, either permanently or subject to re-treatment, in alternative aspects the onset of myopia is delayed.

The term “treatment of myopia” as used herein, and described in certain aspects, refers to the amelioration of at least one symptom of myopia or refers to the retarding of the progression of myopia, for example delaying the progression of scleral stretching, retarding of scleral thinning, or retarding the reducing of scleral strength, for example. The treatment does not need to improve vision, such as improving it to its fullest extent or to normal. In some aspects, the term refers to preventing the progression or slowing the progression of myopia, such as degenerative myopia, for example. In a specific embodiment, the vision stabilizes.

The term “ocular condition” refers to both “ocular deformation condition” as well as other conditions associated with other infectious or cancerous conditions (e.g., tumors), where singlet oxygen is known to have a therapeutic effect.

The term “ocular deformation condition” as used herein refers to a disease or physical change in the eye of a patient which results in a change in the dimension of one or more structures of the eye. In some aspects, this change in dimension causes a change in vision. Specific examples of ocular deformation conditions include degenerative myopia, regular myopia, and scleral staphyloma.

The term “ocular tissue” as used herein refers to a discrete tissue type found in or associated with an eye. In some aspects, the ocular tissue is a structural tissue which establishes and/or maintains the shape of an eye. In other embodiment, the ocular tissue contributes to the vision of an eye. Specific examples of ocular tissues include the sclera, lamina cribosa, and the cornea. The term “ocular media” refers to the ocular tissues that light traverses going from the anterior cornea to the retina. These include the cornea, anterior chamber, lens, and vitreous body.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “orthogonally coupled” refers to the state where the orbitals of the heptamethine linkage of the dye and the orbitals of the optionally substituted cationic heteroaryl ring moiety, preferably the optionally substituted cationic nitrogen-, oxygen, or sulfur-containing heteroaryl ring moiety are orthogonal to one another; i.e., the respective orbitals have limited or no interaction with one another, for example as a consequence of steric crowding. This lack of overlap is what is referred to by the word “orthogonal.” Such “orthogonal coupling” allows for the provision of a longer-lived charge-transfer state, resulting from a “forbidden” relaxation state.

The term “sclera” carries its normal connotation as understood by a person of ordinary skill and refers to the tough, opaque (usually white), outer fibrous coat of the eye, continuous with cornea anteriorly and the optic nerve posteriorly. It comprises collagen, elastic fibers, proteoglycans, cells, and traversing blood vessels and nerves.

As used herein, the terms “substrate” or “organic substrate” are intended to connote both discrete small molecules (sometimes described as “organic compounds”) and oligomers and polymers containing such “aromatic moieties.” The term “aromatic moieties” is intended to refer to those portions of the compounds, pre-polymers (i.e., monomeric compounds capable of polymerizing), oligomers, or polymers having at least one of the indicated aromatic structure. Where shown as structures, the moieties contain at least that which is shown, as well as containing further functionalization, substituents, or both, including but not limited to the functionalization described as “Fn” herein.

By “substituted” as in “substituted hydrocarbyl,” “substituted alkyl,” “substituted aryl,” and the like, as alluded to in some of the aforementioned definitions, is meant that in the hydrocarbyl, alkyl, aryl, heteroaryl, or other moiety, at least one hydrogen atom bound to a carbon (or other) atom is replaced with one or more non-hydrogen substituents. Examples of such substituents include, without limitation: functional groups referred to herein as “Fn,” such as halo (e.g., F, Cl, Br, I), hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₁-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O-CO-alkyl) and C₆-C₂₄ arylcarbonyloxy (—O—CO-aryl)), C₂-C₂₄ alkoxycarbonyl ((CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)NH(C₁-C₂₄ alkyl)), di-(C₁-C₂₄ alkyl)-substituted carbamoyl (—(CO)—N(C₁-C₂₄ alkyl)₂), mono-(C₁-C₂₄ haloalkyl)-substituted carbamoyl (—(CO)—NH(C₁-C₂₄ alkyl)), di-(C₁-C₂₄ haloalkyl)-substituted carbamoyl (—(CO)—N(C₁-C₂₄ alkyl)₂), mono-(C₅-C₂₄ aryl)-substituted carbamoyl (—(CO)—NH-aryl), di-(C₅-C₂₄ aryl)substituted carbamoyl (—(CO)—N(C₅-C₂₄ aryl)₂), di-N—(C₁-C₂₄ alkyl), N—(C₅-C₂₄ aryl)-substituted carbamoyl, thiocarbamoyl (—(CS)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl)₂), mono-(C₅-C₂₄ aryl)substituted thiocarbamoyl (—(CO)—NH-aryl), di-(C₅-C₂₄ aryl)-substituted thiocarbamoyl (—(CO)—N(C₅-C₂₄ aryl)₂), di-N—(C₁-C₂₄ alkyl), N—(C₅-C₂₄ aryl)-substituted thiocarbamoyl, carbamido (—NH—(CO)—NH₂), cyano(-C), cyanato (—O—C≡N), thiocyanato (—S—C═N), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)substituted amino, di-(C₅-C₂₄ aryl)-substituted amino, C₁-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂OH), sulfonate(SO₂O—), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₁-C₂₄ monoalkylaminosulfonyl-SO₂—N(H) alkyl), C₁-C₂₄ dialkylaminosulfonyl-S₂—N(alkyl)₂, C₅-C₂₄ aryl sulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O)₂), phosphinato (P(O)(O—)), phospho (—PO₂), and phosphine (—PH₂); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₂₄ aryl), C₆-C₂₄ alkaryl (preferably C₆-C₁₆ alkaryl), and C₆-C₂₄ aralkyl (preferably C₆-C₁₆ aralkyl). Within these substituent structures, the “alkyl,” “alkylene,” “alkenyl,” “alkenylene,” “alkynyl,” “alkynylene,” “alkoxy,” “aromatic,” “aryl,” “aryloxy,” “alkaryl,” and “aralkyl” moieties may be optionally fluorinated or perfluorinated. Additionally, reference to alcohols, aldehydes, amines, carboxylic acids, ketones, or other similarly reactive functional groups also includes their protected analogs. For example, reference to hydroxy or alcohol also includes those substituents wherein the hydroxy is protected by acetyl (Ac), benzoyl (Bz), benzyl (Bn, Bnl), β-Methoxyethoxymethyl ether (MEM), dimethoxytrityl, [bis-(4-methoxyphenyl)phenylmethyl] (DMT), methoxymethyl ether (MOM), methoxytrityl [(4-methoxyphenyl)diphenylmethyl, MMT), p-methoxybenzyl ether (PMB), methylthiomethyl ether, pivaloyl (Piv), tetrahydropyranyl (THP), tetrahydrofuran (THF), trityl (triphenylmethyl, Tr), silyl ether (most popular ones include trimethylsilyl (TMS), tert-butyldimethylsilyl (TBDMS), tri-iso-propylsilyloxymethyl (TOM), and triisopropylsilyl (TIPS) ethers), ethoxyethyl ethers (EE). Reference to amines also includes those substituents wherein the amine is protected by a BOC glycine, carbobenzyloxy (Cbz), p-methoxybenzyl carbonyl (Moz or MeOZ), tert-butyloxycarbonyl (BOC), 9-fluorenylmethyloxycarbonyl (FMOC), acetyl (Ac), benzoyl (Bz), benzyl (Bn), carbamate, p-methoxybenzyl (PMB), 3,4-dimethoxybenzyl (DMPM), p-methoxyphenyl (PMP), tosyl (Ts) group, or sulfonamide (Nosyl & Nps) group. Reference to substituent containing a carbonyl group also includes those substituents wherein the carbonyl is protected by an acetal or ketal, acylal, or diathane group. Reference to substituent containing a carboxylic acid or carboxylate group also includes those substituents wherein the carboxylic acid or carboxylate group is protected by its methyl ester, benzyl ester, tert-butyl ester, an ester of 2,6-disubstituted phenol (e.g. 2,6-dimethylphenol, 2,6-diisopropylphenol, 2,6-di-tert-butylphenol), a silyl ester, an orthoester, or an oxazoline. Preferred substituents are those identified herein as not or less affecting the silylation chemistries, for example, including those substituents comprising alkyls; alkoxides, aryloxides, aralkylalkoxides, protected carbonyl groups; aryls optionally substituted with F, Cl, —CF₃; epoxides; N-alkyl aziridines; cis- and trans-olefins; acetylenes; pyridines, primary, secondary and tertiary amines; phosphines; and hydroxides.

By “functionalized” as in “functionalized hydrocarbyl,” “functionalized alkyl,” “functionalized olefin,” “functionalized cyclic olefin,” and the like, is meant that in the hydrocarbyl, alkyl, aryl, heteroaryl, olefin, cyclic olefin, or other moiety, at least one hydrogen atom bound to a carbon (or other) atom is replaced with one or more functional groups such as those described herein and above. The term “functional group” is meant to include any functional species that is suitable for the uses described herein. In as used herein, a functional group would necessarily possess the ability to react with or bond to corresponding functional groups on a substrate surface.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

The term “UV-Visible light” as used herein refers to electromagnetic radiation having a wavelength in a range of from about 200 nm to about 750 nm. Individual aspects describing UV-Visible light as an important parameter include those in which the range of wavelengths include one or more ranges encompassing 200 to 250 nm, 250 to 300 nm, 300 to 350 nm, 350 to 400 nm, 400 to 450 nm, 450 to 500 nm, 500 to 550 nm, 550 to 600 nm, 600 to 650 nm, 650 to 700 nm, and/or 700 to 750 nm. The term “near-infrared light” or “NIR light” refers to electromagnetic radiation in a range of from about 750 nm to about 1400 nm. Individual aspects describing NIR light as am important parameter include those in which the range of wavelengths include one or more ranges encompassing 750 to 800 nm, 800 to 850 nm, 850 to 900 nm, 900 to 950 nm, 950 to 1000 nm, 1000 to 1050 nm, 1050 to 1100 nm, 1100 to 1200 nm, 1200 to 1300 nm, and/or 1300 to 1400 nm. It should be appreciated that reference to the irradiation by NIR light or by a wavelength of near-infrared (NIR) light, as used herein, is intended to connote that the irradiation includes only, or practically only, NIR light; that is, the irradiating light is devoid of any UV-Visible light wavelength capable of activating a NIR dye to generate singlet oxygen in the presence of oxygen.

The following listing of embodiments is intended to complement, rather than displace or supersede, the previous descriptions.

Embodiment 1. A method of altering a mechanical and/or chemical property of a tissue, the method comprising irradiating a near-infrared-(NIR) photoactive direct treatment composition with near-infrared light in the presence of oxygen;

wherein the near-infrared (NIR) photoactive direct treatment composition comprises a near-infrared dye that generates singlet oxygen when irradiated with near-infrared light in the presence of oxygen; and

wherein the near-infrared (NIR) photoactive direct treatment composition is preferably adjacent to (contacts) or has permeated the tissue.

In certain Aspects of this Embodiment, the altering or change of the mechanical and/or chemical property refers to a desirable improvement of the mechanical and/or chemical property (e.g., strengthening or stiffening of the tissue).

In certain Aspects of this Embodiment, the irradiating results in a change in the mechanical and/or chemical property of a tissue in the patient.

In certain Aspects of this Embodiment, the method may comprise a method of treating tissue degeneration in a patient. In certain other Aspects of this Embodiment, the method may comprise a method of preventing or slowing the progress of tissue degeneration in a patient.

In certain Aspects of this Embodiment, the tissue is a collagen-containing tissue. In certain Aspects of this Embodiment, the method is operative in vivo in a patient. In other Aspects of this Embodiment, the method is operative ex vivo.

In certain Aspects of this Embodiment, the near-infrared (NIR) photoactive direct treatment composition is independently adjacent to (contacts) or has permeated the tissue. In certain Aspects of this Embodiment, the near-infrared (NIR) photoactive direct treatment composition is in contact with deuterated water (D₂O), or other physiologically acceptable deuterated fluid. In other Aspects of this Embodiment, the near-infrared (NIR) photoactive direct treatment composition is in contact with a physiologically acceptable fluorinated or perfluorinated fluid.

In certain Aspects of this Embodiment, the irradiating the near-infrared-(NIR) photoactive direct treatment composition with near-infrared light is done using a laser. In other Aspects, the laser delivers the near-infrared light as any of the wavelength ranges or individual wavelengths disclosed herein. In other Aspects, the laser delivers the near-infrared light continuously. In other Aspects, the laser delivers the near-infrared light intermittently or in pulses.

In certain Aspects of this Embodiment, the irradiating is done in the presence of deuterated water (D₂O), or other physiologically acceptable deuterated fluid.

In certain Aspects of this Embodiment, the oxygen is present or presented to the tissue and/or the near-infrared-(NIR) photoactive direct treatment composition via dissolved oxygen in the tissue, ocular fluid, blood vessels, or an added aqueous fluid (including deuterated aqueous fluid). In other Aspects of this Embodiment, the concentration of oxygen is present above natural levels by the deliberate addition of oxygen to the site of the irradiation. In still other Aspects, the deliberate addition of oxygen is made by flowing oxygen to the site of the irradiation.

In certain Aspects of this Embodiment, the irradiating the near-infrared-(NIR) photoactive direct treatment composition with near-infrared light to generate singlet oxygen operates by the crosslinking of the tissue.

In certain Aspects of this Embodiment, the irradiating the near-infrared-(NIR) photoactive direct treatment composition with near-infrared light is provided through the front of the eye (through the retina). The near-infrared light may also be provided through a patterned mask. In certain Aspects of this Embodiment, the near-infrared light is provided through the retina.

In certain Aspects of this Embodiment, the irradiating with near-infrared light is directed to a region of the tissue identified by diagnostic imaging. Such diagnostic imaging may independently include ultrasound imaging, optical coherence tomography (OCT) imaging, OCT angiographic imaging, OCT Doppler imaging, or magnetic resonance imaging (MRI). These imaging methods can also be used to map regions that are avoided during the NIR irradiation.

Embodiment 2. The method of Embodiment 1, wherein the mechanical and/or chemical property is tensile strength, compression strength, flexural strength, modulus, elongation, or toughness of the tissue. The methods involving the treatment of these properties are considered independent Aspects, as if listed separately.

Embodiment 3. The method of Embodiment 1 or 2, wherein the tissue is an ocular tissue.

In certain Aspects of this Embodiment the tissue is an ocular tissue and the method treats a symptom of the ocular deformation condition by strengthening the ocular tissue, stabilizing the ocular tissue shape, changing the shape of the ocular tissue, or a combination thereof.

In certain Aspects of this Embodiment, the method may comprise a method of treating ocular degeneration in a patient.

In certain Aspects of this Embodiment, the tissue is an ocular tissue and irradiating the near-infrared (NIR) photoactive direct treatment composition independently directly treats or directly reduces the risk of the ocular deformation condition.

Embodiment 4. The method of Embodiment 3, wherein the ocular tissue includes at least a portion of a cornea and/or a sclera. In certain Aspects of this Embodiment, the ocular tissue comprises the posterior portion of the cornea and/or the sclera. In other Aspects of this Embodiment, the ocular tissue comprises one or more anterior (peripheral) portions of the cornea and/or the sclera. In other Aspects of this Embodiment, the ocular tissue comprises the portion of the sclera around the optic nerve.

Embodiment 5. The method of Embodiment 3, wherein the ocular tissue includes at least a portion of a lamina cribrosa.

Embodiment 6. The method of any one of Embodiments 1 to 5, wherein the patient has or is at risk of developing an ocular deformation condition comprising one or more of degenerative myopia, regular myopia, scleral staphyloma, keratoconus (including progressive keratoconus and other ectatic corneal conditions), or glaucoma. In certain Aspects of this Embodiment, the method reduces the progression of the ocular deformation condition.

Embodiment 7. The method of any one of Embodiments 1 to 6, further comprising administering the near-infrared (NIR) photoactive direct treatment composition to the tissue of the patient, either topically or by intravenous or ocular injection.

Embodiment 8. A method of treating bacterial or fungal infections or cancer cells or tumors in the eye, the method comprising irradiating a near-infrared-(NIR) photoactive direct treatment composition with near-infrared light in the presence of oxygen;

wherein the near-infrared (NIR) photoactive direct treatment composition comprises a near-infrared dye that generates singlet oxygen when irradiated with near-infrared light in the presence of oxygen; and

wherein the near-infrared (NIR) photoactive direct treatment composition is preferably adjacent to (contacts) or has permeated the tissue.

The localized generation of singlet oxygen is useful in the treatment of these conditions.

In certain Aspects of this Embodiment, the tissue is a collagen-containing tissue. In certain Aspects of this Embodiment, the method is operative in vivo in a patient. In other Aspects of this Embodiment, the method is operative ex vivo.

In certain Aspects of this Embodiment, the method is directed to corneal and other ocular infections. In certain Aspects of this Embodiment, the method is directed to keratitis. In certain Aspects of this Embodiment, the method is directed to bacterial keratitis. In certain Aspects of this Embodiment, the method is directed to fungal keratitis. In certain Aspects of this Embodiment, the method is directed to deep corneal bacterial keratitis.

In certain Aspects of this Embodiment, the method is directed to ocular melanoma. In certain Aspects of this Embodiment, the method is directed to choroidal melanoma.

In certain Aspects of this Embodiment, the near-infrared (NIR) photoactive direct treatment composition is independently adjacent to (contacts) or has permeated the tissue. In certain Aspects of this Embodiment, the near-infrared (NIR) photoactive direct treatment composition is dissolved or suspended in or in contact with deuterated water (D₂O), or other physiologically acceptable deuterated fluid. In other Aspects of this Embodiment, the near-infrared (NIR) photoactive direct treatment composition is in contact with a physiologically acceptable fluorinated or perfluorinated fluid.

In certain Aspects of this Embodiment, the irradiating with near-infrared light is done using a laser. In other Aspects, the laser delivers the near-infrared light as any of the wavelength ranges or individual wavelengths disclosed herein. In other Aspects, the laser delivers the near-infrared light continuously. In other Aspects, the laser delivers the near-infrared light intermittently or in pulses.

In certain Aspects of this Embodiment, the irradiating is done in the presence of deuterated water (D₂O), or other physiologically acceptable deuterated fluid.

In certain Aspects of this Embodiment, the oxygen is present or presented to the tissue and/or the near-infrared-(NIR) photoactive direct treatment composition via dissolved oxygen in the tissue, ocular fluid, blood vessels, or an added aqueous fluid (including deuterated aqueous fluid). In other Aspects of this Embodiment, the concentration of oxygen is present above natural levels by the deliberate addition of oxygen to the site of the irradiation. In still other Aspects, the deliberate addition of oxygen is made by flowing oxygen to the site of the irradiation.

In certain Aspects of this Embodiment, the irradiation of the near-inrfared sensitizer with the near-infrared light is provided through the front of the eye (through the retina). The near-infrared light may also be provided through a patterned mask.

In certain Aspects of this Embodiment, the irradiating with near-infrared light is directed to a region of the tissue identified by diagnostic imaging. Such diagnostic imaging may independently include ultrasound imaging, optical coherence tomography (OCT) imaging, OCT Doppler imaging, OCT angiography, or magnetic resonance imaging (MRI).

Embodiment 9. The method of any one of Embodiment 1 to 8, wherein the near-infrared-(NIR) photoactive direct treatment composition comprises a near-infrared (NIR) absorbing dye having a heptamethine linkage

Embodiment 10. The method of Embodiment 9, wherein the near-infrared (NIR) absorbing dye comprises a cyanine structure, a pyrylium structure, or a thiopyrylium structure, or a combination thereof. Each of these types of structures are considered independent Aspects of this Embodiment.

Embodiment 11. The method of any one of Embodiments 1 to 10, wherein the near-infrared (NIR) absorbing dye comprises a cyanine dye structure.

In certain Aspects of this Embodiment, the near-infrared (NIR) absorbing dye comprising the cyanine structure, include any and all such heptamethine dyes (albeit without the optionally substituted cationic nitrogen-, oxygen, or sulfur-containing heteroaryl ring moiety) that are described in U.S. Pat. Nos. 4,464,383; 5,563,028; 5,633,390; 5,973,158; 6,072,059; 6,515,811; 6,673,943; 9,610,370; and 10,280,307; each of which is incorporated by reference herein at least for its descriptions of dye portion of the claimed compounds (including backbones, substituents, and substitution patterns) and for its teachings of the methods of making and using the same.

Embodiment 12. The method of any one of Embodiments 9 to 11, wherein the near-infrared (NIR) absorbing dye comprises a structure of:

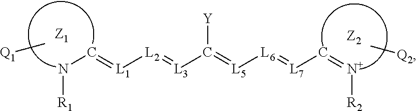

or a rotational or conformational isomer or a salt thereof; wherein

L₁, L₂, L₃, L₅, L₆, and L₇ are substituted or unsubstituted methines, wherein the optional substituents are independently C₁₋₆ alkyl or C₂₋₆ alkenyl; or L₁ and L₃, or L₃ and L₅, or L₅ and L₇ may be linked with a C₂₋₄ alkylene or C₂₋₄ alkenylene substituent to form a 5- to 7-membered ring;

each of Z¹ and Z² is independently a five- or six-membered nitrogen-containing heterocyclic ring, optionally fused to another aryl or heteroaryl ring;

each of Q₁ and Q₂ is independently H or a substituent positioned on the five- or six-membered nitrogen-containing heterocyclic ring and/or the optionally fused aryl or heteroaryl ring, each optional substituent comprising an optionally substituted C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof, —(C₀₋₁₂alkyl)borate,

R₁ and R₂ is independently C₁₋₁₂ alkyl, —[CH₂—CH₂—O—]₁₋₆R¹⁰, —(C₀₋₁₂alkyl)amino acid residue, or a 5- or 6-member ringed aryl or heteroaryl, each of which may be optionally substituted with one or more —(C₀₋₁₂alkyl)(SO₃)—R¹⁰ or a salt thereof, —(C₀₋₁₂alkyl)(SO₄)—R¹⁰ or a salt thereof, —(C₀₋₁₂alkyl)(PO₄)—R¹⁰ or a salt thereof, —(C₀₋₁₂alkyl)OR¹⁰, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof or —(C₀₋₁₂alkyl)borate or borate ester;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substitutedaryloxy, optionally substituted heteroaryloxy, halogen, or optionally substituted cationic heteroaryl moiety.

In certain Aspects of this Embodiment, Y is H, or an optionally substituted amine, optionally substituted alkyl, optionally substituted alkoxy, optionally substituted aryl, optionally substituted heteroaryl, optionally substituted aryloxy, optionally substituted heteroaryloxy, and halogen, as set forth elsewhere herein.

In certain Aspects of this Embodiment, Y is an optionally substituted cationic heteroaryl moiety. In this context, the optionally substituted cationic heteroaryl moiety is more fully set forth elsewhere herein.

Likewise, it should be appreciated that while Q₁, Q₂, R₁, and R₂ are defined in terms of specific optional substituents, and Y is defined merely as “optionally substituted,” in some Aspects of this Embodiment, the optional substituents may also include those defined elsewhere herein as Fn. In this regard, any one or more of these Fn substituents is considered to be selected independently, as if listed individually.

It should be appreciated that while Y is shown here as being in the L₄ position (i.e., between the L₃ and L₅ methines), and is preferably positioned there, in other Aspects of this Embodiment, Y can be alternatively positioned on any of the L₁, L₂, L₃, L₄, L₅, L₆, or L₇ positions. Likewise, other Aspects of this Embodiment include all geometric and rotational isomers of the provided structure.

In some Aspects of this Embodiment, Z₁ and Z₂ are the same. In other Aspects of this Embodiment, Z₁ and Z₂ are different. It should also be understood throughout that reference to a five- or six-membered nitrogen-containing ring includes these five- and six-membered nitrogen-containing rings as separated Aspects of any Embodiment cited herein.

In preferred Aspects of this Embodiment, the heptamethine linkage is directly bonded to Y.

Embodiment 13. The method of Embodiment 12, wherein the five- or six-membered nitrogen-containing heterocyclic ring of Z₁ and Z₂ independently comprise a pyrrole ring, imidazole ring, isothiazole ring, isoxazole ring, oxadiazole ring, oxazole ring, pyrazole ring, pyrimidyl, thiazole ring, selenazole ring, thiadiazole ring, triazole ring, or a pyridine ring. Again, it is understood that in certain Aspects of this Embodiment, Z₁ and Z₂ are the same. In other Aspects, Z₁ and Z₂ are different.

Embodiment 14. The method of Embodiment 12 or 13, wherein the five- or six-membered nitrogen-containing heterocyclic ring of Z₁ and Z₂ is independently fused to a phenyl, naphthyl, pyridinyl, quinolinyl, quinoxalinyl, N-alkyl-benzoindolenine, dibenzofuranyl, or dibenzothiophenyl.

Embodiment 15. The method of Embodiment 12 or 13, wherein Z₁ and Z₂ independently comprise a benzimidazole ring, benzindole ring, benzoindolenine ring, benzoxazole ring, benzothiazole ring, furopyrrole ring, imidazole ring, imidazoquinoxaline ring, indolenine ring, indolizine ring, isoxazole ring, naphthimidazole ring, naphthothiazole ring, naphthoxazole ring, oxazolocarbazole ring, oxazole ring, oxazolodibenzofuran ring, pyrrolopyridine ring, pyridine ring, quinoline ring, quinoxaline ring, thiazole ring, or naphthoimidazole ring.

Embodiment 16. The method of any one of Embodiments 12 to 15, wherein the methines not bonded to Y are otherwise not substituted. For example, in some Aspects of this Embodiment, where Y is in the L₄ position, L₁=L₂=L₃=L₅=L₆=L₇=CH. In other Aspects, where Y is in the L₁ position, L₂=L₃=L₄=L₅=L₆=L₇=CH. In other Aspects, where where Y is in the L₂ position, L₁=L₃=L₄=L₅=L₆=L₇=CH. In other Aspects, where where Y is in the L₃ position, L₁=L₂=L₄=L₅=L₆=L₇=CH.

Embodiment 17. The method of any one of Embodiments 12 to 15, wherein one of L₁ and L₃, or L₂ and L₄, or L₃ and L₅, or L₄ and L₆, or L₅ and L₇ are linked with a C₂₋₄ alkylene substituent to form a 5- to 7-membered ring. Each of these Aspects of this Embodiment is considered independently and combinable with any Aspect or Embodiment of the preceding or following Embodiments.

In an exemplary Aspect of this Embodiment, the near-infrared (NIR) absorbing dye comprises a structure of:

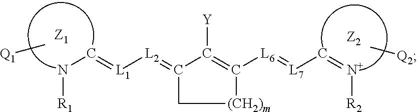

or rotational or conformational isomer or a salt thereof, where L₁, L₂, L₃, L₄, L₅, L₆, L₇, Q₁, R₁, Y, Z₁, are defined in any of the definitions as set forth elsewhere herein in any combination or permutations and m is 1, 2, or 3.

Embodiment 18. The method of any one of Embodiments 12 to 17, wherein the near-infrared (NIR) absorbing dye comprises a structure of:

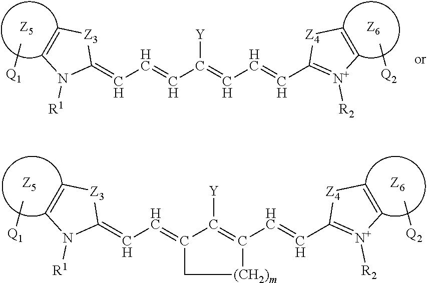

or a rotational or conformational isomer or a salt thereof; wherein

each of Z₃ and Z₄ is independently —CR¹¹R₁₂, —NR¹¹, —O—, —S— or —Se— (each of Z₃ and Z₄ is independently preferably —CR¹¹R₁₂, —NR¹¹, —O— or —S—, each of Z₃ and Z₄ is independently more preferably is —CR¹¹R₁₂, —O— or —S, each of Z₃ and Z₄ is independently further preferably is ——CR¹¹R₁₂ or —O—, and each of Z₃ and Z₄ is independently most preferably —CR¹¹R₁₂);

each of Z₅ and Z₆ is independently preferably phenyl, naphthyl, pyridinyl, quinolinyl, quinoxalinyl, N-alkyl-benzoindolenine, dibenzofuranyl, or dibenzothiophenyl,

each of R¹¹ and R¹² is independently a C₁₋₆ alkyl, preferably methyl; and

Q₁ and Q₂ are independently, preferably H, —COOH or a salt thereof, or —SO₃H or a salt thereof.

Embodiment 19. The method of any one of claims 12 to 18, wherein the near-infrared (NIR) absorbing dye comprises a structure of:

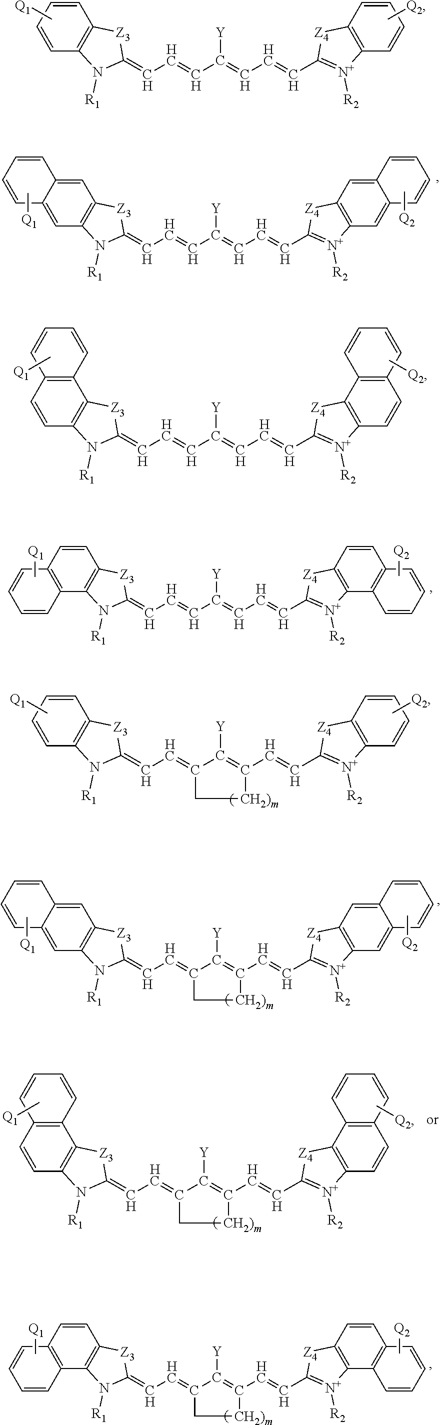

or a rotational or conformational isomer or a salt form thereof;

wherein each of Z₃ and Z₄ is independently —CR¹¹R₁₂, —NR¹¹, —O—, —S— or —Se— (each of Z₃ and Z₄ is independently preferably —CR¹¹R₁₂, —NR¹¹, —O— or —S—, each of Z₃ and Z₄ is independently more preferably is —CR¹¹R₁₂, —O— or —S, each of Z₃ and Z₄ is independently further preferably is —CR¹¹R¹² or —O—, and each of Z₃ and Z₄ is independently most preferably —CR¹¹R¹²);

each of R¹¹ and R¹² is independently a C₁₋₆ alkyl, preferably methyl;

m=1, 2, or 3; and

Q₁ and Q₂ are independently, preferably H, —COOH or a salt thereof, or —SO₃H or a salt thereof.

In certain independent Aspects of this Embodiment, the fused naphthalene moiety may be replaced with an optionally substituted quinolinyl, quinoxalinyl, N-alkyl-benzoindolenine, dibenzofuranyl, or dibenzothiophenyl ring.

Embodiment 20. The method of any one of Embodiments 12 to 19, wherein the near-infrared (NIR) absorbing dye comprises a structure of:

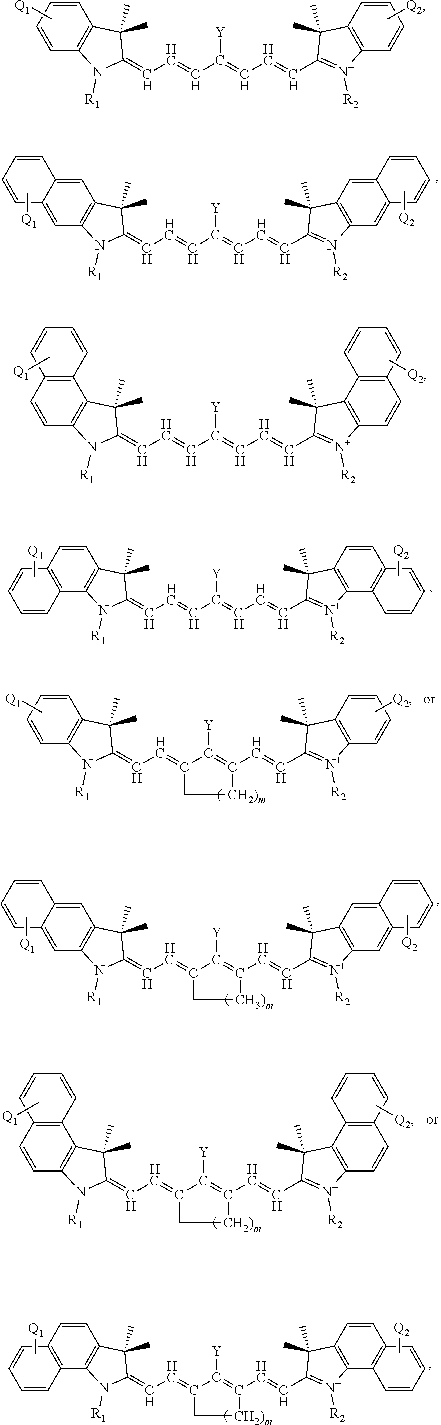

or a rotational or conformational isomer or a salt form thereof;

where R₁ and R₂ are independently —(C₁₋₁₂alkyl)(SO₃)H or a salt thereof or —(C₁₋₂alkyl)COOH or a salt thereof. Each of these structures represent an independent Aspect of this Embodiment.

In certain independent Aspects of this Embodiment, the fused naphthalene moiety may be replaced with an optionally substituted quinolinyl, quinoxalinyl, N-alkyl-benzoindolenine, dibenzofuranyl, or dibenzothiophenyl ring.

Embodiment 21. The method of claim 12, wherein the near-infrared (NIR) absorbing dye comprises a structure or rotational or conformation isomer of:

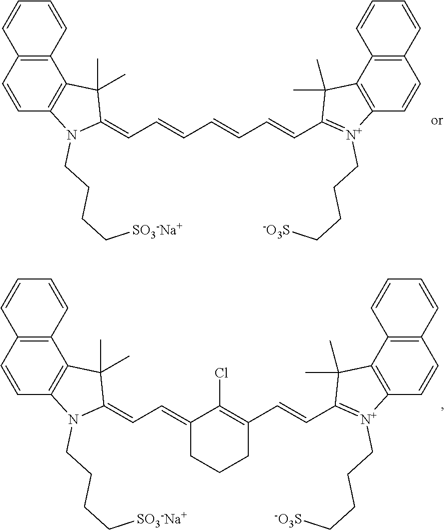

or a rotational or conformational isomer or alternative salt form thereof. In certain Aspects of this Embodiment, these structures are excluded from the methods.

Embodiment 22. The method of any one of Embodiment 1 to 10, wherein the near-infrared (NIR) absorbing dye comprises a pyrylium dye or a thiopyrylium dye. In certain Aspects of this Embodiment, the near-infrared (NIR) absorbing dye comprising the pyrylium dye or the thiopyrylium dye includes any and all such heptamethine dyes that are described in U.S. Pat. No. 4,283,475 that are incorporated by reference for its teachings of these types of dyes, and the ability to functionalize and make these dyes.

Embodiment 23. The method of Embodiment 22, wherein the near-infrared (NIR) absorbing dye comprises a pyrylium or thiopyrylium structure of:

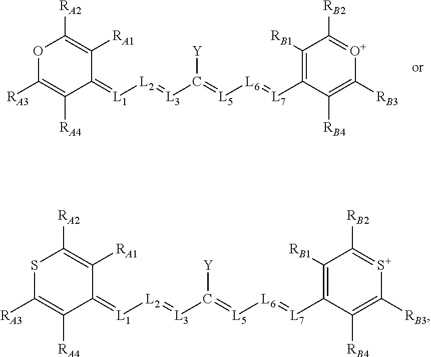

respectively, or a rotational or conformational isomer or a salt thereof;

wherein

L₁, L₂, L₃, L₅, L₆, and L₇ are substituted or unsubstituted methines, wherein the optional substitutents are independently C₁₋₆ alkyl or C₂₋₆ alkenyl; or L₁ and L₃, or L₃ and L₅, or L₅ and L₇ may be linked with C₂₋₄ alkylene or C₂₋₄ alkenylene substituents;

R_{A1}, R_{A2}, R_{A3}, R_{A4}, R_{B1}, R_{B2}, R_{B3}, and R_{B4} are each independently H, deutrium, or tritium, an C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo (fluoro, chloro, bromo, iodo), nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R₁₀ or a salt thereof, —(C₀₋₁₂alkyl)COOR¹⁰ or a salt thereof, —(C₀₋₁₂alkyl)CON(R¹⁰)₂ or a salt thereof, —(C₀₋₁₂alkyl)N(R¹⁰)₂ or a salt thereof, —(C₀₋₁₂alkyl)borate;

n is independently 0, 1, 2, 3, or 4, preferably 2;

R¹⁰ is independently H or C₁₋₆ alkyl; and

Y is H, or an optionally substituted amine, optionally substituted alkyl, optionally substituted alkoxy, optionally substituted aryl, optionally substituted heteroaryl, optionally substituted aryloxy, optionally substituted heteroaryloxy, halogen, or optionally substituted cationic nitrogen-containing heteroaryl moiety.

In certain Aspects of this Embodiment, Y is H, or an optionally substituted amine, optionally substituted alkyl, optionally substituted alkoxy, optionally substituted aryl, optionally substituted heteroaryl, optionally substituted aryloxy, optionally substituted heteroaryloxy, and halogen, as set forth elsewhere herein.

In certain Aspects of this Embodiment, Y is an optionally substituted cationic heteroaryl moiety. In this context, the optionally substituted cationic heteroaryl moiety is more fully set forth elsewhere herein.

Likewise, it should be appreciated that while Y is defined merely as “optionally substituted,” the optional substituents may also include those defined elsewhere herein as Fn. In this regard, any one or more of these Fn substituents is considered to be selected independently, as if listed individually. Also, R_{A1}, R_{A2}, R_{A3}, R_{A4}, R_{B1}, R_{B2}, R_{B3}, and R_{B4} may also independently be any one or more of these Fn substituents.

In some Aspects of this Embodiment, Z₁ and Z₂ are the same. In other Aspects of this Embodiment, Z₁ and Z₂ are different.

Embodiment 24. The method of Embodiment 14, wherein the methines not bonded to Y are otherwise not substituted. For example, in some Aspects of this Embodiment, where Y is in the L₄ position, L₁=L₂=L₃=L₅=L₆=L₇=CH. In other Aspects, where Y is in the L₁ position, L₂=L₃=L₄=L₅=L₆=L₇=CH. In other Aspects, where Y is in the L₂ position, L₁=L₃=L₄=L₅=L₆=L₇=CH. In other Aspects, where Y is in the L₃ position, L₁=L₂=L₄ L₅ L₆ L₇=CH.

In an exemplary Aspect of this Embodiment, the near-infrared (NIR) absorbing dye comprises a structure of:

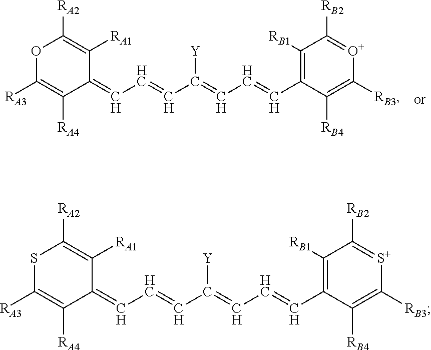

or a rotational or conformational isomer or a salt thereof.

Embodiment 25. The method of Embodiment 23, wherein one of L₁ and L₃, or L₂ and L₄, or L₃ and L₅, or L₄ and L₆, or L₅ and L₇ are linked with a C₂₋₄ alkylene sub stituent to form a 5- to 7-membered ring. Each of these Aspects of this Embodiment is considered independently and combinable with any Aspect or Embodiment of the preceding or following Embodiments.

In an exemplary Aspect of this Embodiment, the near-infrared (NIR) absorbing dye comprises a structure of:

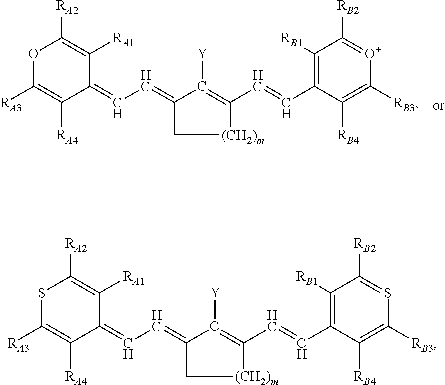

or a rotational or conformational isomer or a salt thereof; where m is 1, 2, or 3.

Embodiment 26. The method of any one of Embodiment 23 to 25, wherein R_{A1}, R_{A4}, R_{B1}, and R_{B4} are H, or an isotope thereof, and R_{A2}, R_{A3}, R_{B2}, and R_{B3} are aryl, heteroaryl, or branched alkyl preferably phenyl, pyridinyl, or tert-butyl.

Embodiment 27. The method of any one of Embodiments 12 to 26, wherein Y is H, or an optionally substituted amine, optionally substituted alkyl, optionally substituted alkoxy, optionally substituted aryl, optionally substituted heteroaryl, optionally substituted aryloxy, optionally substituted heteroaryloxy, or halogen.

Embodiment 28. The method of any one of claims 12 to 26, wherein Y is an optionally substituted cationic heteroaryl ring moiety, preferably an optionally substituted cationic nitrogen-, oxygen, or sulfur-containing heteroaryl ring moiety. It is to be understood that each of the cationic nitrogen-, oxygen, or sulfur-containing heteroaryl moieties is considered an independent Aspect of this Embodiment. In the context of the cationic heteroaryl ring moiety, preferably an optionally substituted cationic nitrogen-, oxygen, or sulfur-containing heteroaryl ring moiety, the cationic charge is distributed as a formal charge within the ring structure of the heteroaryl ring moiety, as opposed to residing on one or more of the optional substituents.

In certain Aspects of this Embodiment, this heteroaryl ring moiety is directly bonded to the heptamethine linkage; i.e., no additional linking groups. In certain Aspects of this Embodiment, the optionally substituted cationic nitrogen-containing heteroaryl ring is bonded to the heptamethine linkage by a C—C bond or a C—N bond. In certain Aspects of this Embodiment, the optionally substituted cationic oxygen- or sulfur-containing heteroaryl ring is bonded to the heptamethine linkage by a C—C bond.

In other preferred Aspects of this Embodiment, the heptamethine linkage is orthogonally coupled to the optionally substituted cationic heteroaryl ring moiety, preferably the optionally substituted cationic nitrogen-, oxygen, or sulfur-containing heteroaryl ring moiety.

In some Aspects of this Embodiment, the optionally substituted cationic heteroaryl ring moiety, preferably the optionally substituted cationic nitrogen-, oxygen, or sulfur-containing heteroaryl ring moiety is characterized as a charge-transfer partner of the near-infrared (NIR) absorbing dye.

In the context of this Embodiment and throughout, the term “orthogonally coupled” refers to the state where the orbitals of the heptamethine linkage of the dye and the orbitals of the optionally substituted cationic heteroaryl ring moiety, preferably the optionally substituted cationic nitrogen-, oxygen, or sulfur-containing heteroaryl ring moiety are orthogonal to one another; i.e., the respective orbitals have limited or no interaction with one another, for example as a consequence of steric crowding. This lack of overlap is what is referred to by the word “orthogonal.” Such “orthogonal coupling” allows for the provision of a longer-lived charge-transfer state, resulting from a “forbidden” relaxation state.

Embodiment 29. The method of Embodiment 12 to 28, wherein the optionally substituted cationic heteroaryl moiety comprises an optionally substituted acridinium, benzoxazolium, benzothiazolium, imidazolium, isoxazolium, isoquinolinium, isothiazolium, naphthoimidazolium, naphthothiazolium, naphthoxazolium, oxazolium, pyrazinium, pyrazolium, pyridimium, pyridinium, quinolinium, tetrazinium, tetrazolium, thiazolium, triazinium, triazolium, benzopyrazinium, benzopyridimium, benzopyridinium, naphthopyrazinium, naphthopyridimium, benzopyridinium, benzotriazinium, naphthotriazinium moiety, pyrylium, chromenylium, xanthylium moiety, thiopyrylium, thiochromenylium, or thioxanthylium moiety.

In certain Aspects of this Embodiment, the optional substituents comprise any one or more of the functional group Fn a set forth elsewhere herein. In this regard, any one or more of these Fn substituents is considered to be selected independently, as if listed individually.

Embodiment 30. The method of Embodiment 12 to 29, wherein the optionally substituted cationic heteroaryl moiety comprises an optionally substituted structure of:

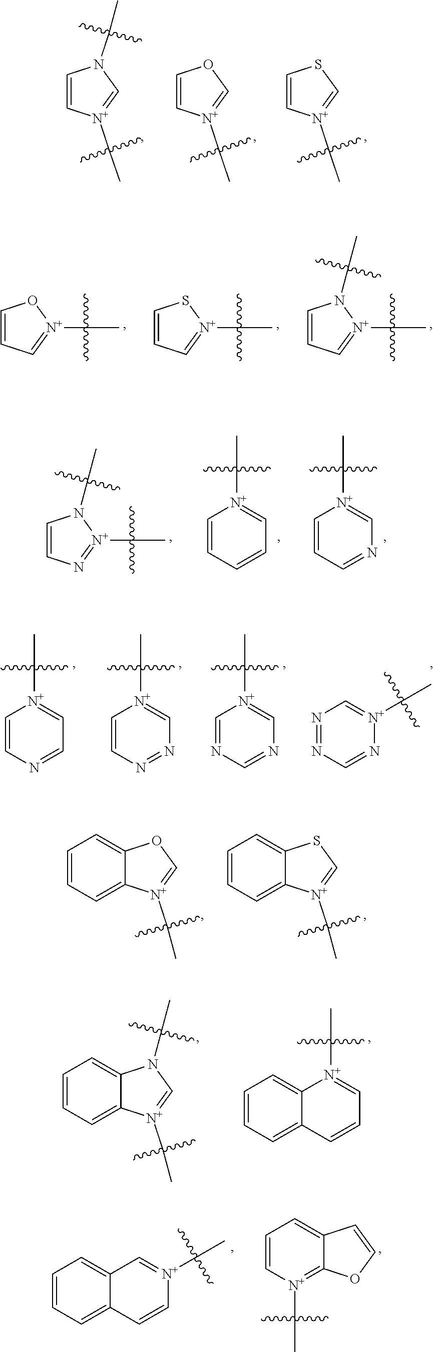

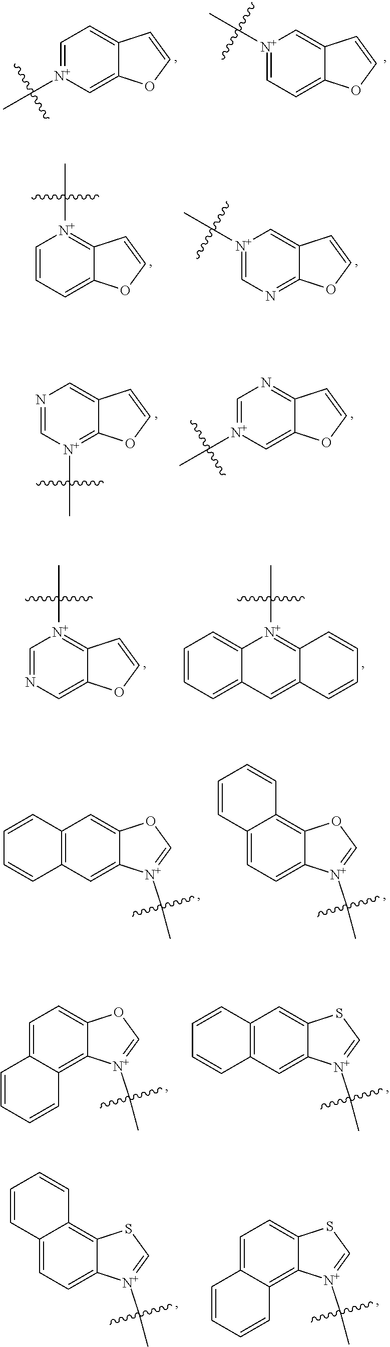

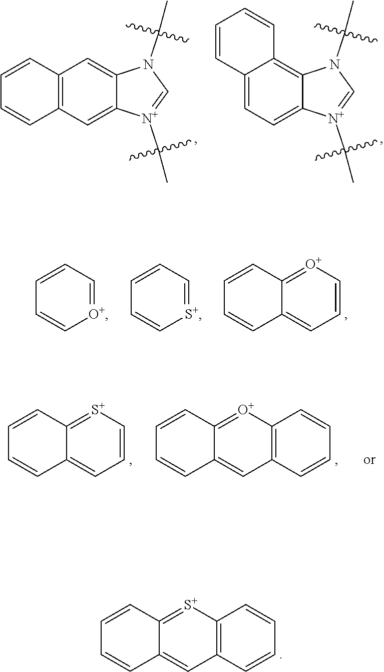

Embodiment 31. The method of any one of Embodiments 10 to 30, wherein the near-infrared (NIR) absorbing dye comprises at least one cationic group and has a net neutral or net positive charge, wherein the associated cationic group, groups or moieties are charge balanced by anionic counter ions. In certain Aspects of this Embodiment, the anionic counter ions are halide anions (e.g., fluoride, chloride, bromide, and/or iodide), or other inorganic anions (e.g., perchlorate, tetrafluoroborate, hexafluorophosphate, sulfate, hydrogensulfate and/or nitrate) or organic anions (e.g., organic anions such as trifluoroacetate, trichloroacetate, triflate, mesylate, and/or p-toluenesulfonate ions).

In some Aspects of this Embodiment, the near-infrared (NIR) absorbing dye may also have least some of the associated cationic groups or moieties that are internally charged balanced.

Where one or more substituents are anionic (for example, carboxylate or sulfonate anions), they may have associated counter cations, such as alkali metal cations, such as Li⁺, Na⁺, or K⁺. The choice of counter cations or anions should not be limited.

Embodiment 32. The method of any one of Embodiments 1 to 31, wherein the near-infrared (NIR) absorbing dye comprises. is substituted with, or is conjugated to at least one isotope of carbon, chlorine, fluorine, hydrogen, iodine, nitrogen, or oxygen enriched above its natural abundance. In certain Aspects of this Embodiment, the isotope is a radioisotope. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S.

In some Aspects of this Embodiment, the degree of enrichment is at least 5 time, at least 10 times, at least 100 time, or at least 1000 times (depending on the nature of the isotope and its natural abundance) above its natural abundance up to completely substituted in that isotope.

Embodiment 33 The method of any one of Embodiments 1 to 32, wherein the irradiating is done with a light having a wavelength in a range of from 750 nm to 800 nm, from 800 nm to 850 nm, from 850 nm to 900 nm, from 900 nm to 950 nm, from 950 nm to 1000 nm, from 1000 nm to 1050 nm, from 1050 nm to 1100 nm, from 1100 nm to 1150 nm, from 1150 nm to 1200 nm, from 1200 nm to 1250 nm, from 1250 to 1300 nm, from 1300 to 1350 nm, from 1350 nm to 1400 nm, or in a range comprising two of more of these foregoing ranges.

Embodiment 34. The method of any one of Embodiments 1 to 33, wherein the near-infrared (NIR) absorbing dye exhibits a local λₘₐₓ for light absorption in a range of from 750 nm to 1400 nm. In independent Aspects of this Embodiment, this range can be defined in terms of from 750 nm to 800 nm, from 800 nm to 850 nm, from 850 nm to 900 nm, from 900 nm to 950 nm, from 950 nm to 1000 nm, from 1000 nm to 1050 nm, from 1050 nm to 1100 nm, from 1100 nm to 1150 nm, from 1150 nm to 1200 nm, from 1200 nm to 1250 nm, from 1250 to 1300 nm, from 1300 to 1350 nm, from 1350 nm to 1400 nm, or in a range comprising two of more of these foregoing ranges, for example from 800 nm to 1100 nm.

Embodiment 35. The method of any one of Embodiments 1 to 34, when the near-infrared dye generates singlet oxygen, when irradiated in the presence of O₂ at a wavelength in a range of from 750 nm to 1400 nm. In independent Aspects of this Embodiment, this range can be defined in terms of from 750 nm to 800 nm, from 800 nm to 850 nm, from 850 nm to 900 nm, from 900 nm to 950 nm, from 950 nm to 1000 nm, from 1000 nm to 1050 nm, from 1050 nm to 1100 nm, from 1100 nm to 1150 nm, from 1150 nm to 1200 nm, from 1200 nm to 1250 nm, from 1250 to 1300 nm, from 1300 to 1350 nm, from 1350 nm to 1400 nm, or in a range comprising two of more of these foregoing ranges, for example from 800 nm to 1100 nm.

Embodiment 36. The method of any one of Embodiments 1 to 35, wherein the near-infrared (NIR) photoactive direct treatment composition further comprises or is associated with a biocompatible solvent. In certain Aspects of this Embodiment, the near infrared dye is dissolved or suspended in the solvent. In certain Aspects, the solvent contacts or wets the near-infrared (NIR) photoactive dye. In certain Aspects, the near-infrared (NIR) photoactive dye is absorbed (permeated) into or adhered to the tissue and the solvent contacts or wets the dye and/or the tissue. In certain Aspects of this Embodiment, the solvent is biocompatible with human patients, including tissues and biological systems. In other independent Aspects of this Embodiment, the solvent is optically transparent in the UV-VIS and near-infrared range of the optical spectrum. In other independent Aspects of this Embodiment, the solvent provides an oxygen solubility greater than H₂O under comparable oxygen partial pressures.

Embodiment 37. The method of Embodiment 36, wherein the solvent comprises a deuterated solvent. In certain Aspects of this Embodiment, the deuterated solvent is or comprises deuterated dimethyl sulfoxide, methanol, ethanol, tetrahydrofuran, or water.

Embodiment 38. The method of Embodiment 36 or 37, wherein the solvent comprises D₂O.

Embodiment 39. The method of any one of Embodiments 36 to 38, wherein the solvent is a fluorinated or perfluorinated solvent. In certain Aspects of this Embodiment, the solvent is fluorinated. In other Aspects of this Embodiment, the solvent is perfluorinated.

In certain Aspects of this Embodiment, such solvents may include or comprise perfluorodecalin, perfluorooctyl bromide, Perflubron-FDA (approved in US as contrast medium), perfluorodecyl bromide, perfluoro-1,3-dimethylcyclohexane, perfluoro(tert-butylcyclohexane), tertbutylperfluorocyclohexane, perfluoro-N-(4-methylcyclohexyl)-piperidine, perfluoromethylodecalin, dodecafluoropentane, Perlenapent, perfluoro-15-crown-5-ether, perfluorotributylamine, perfluorotripropylamine, perfluoro-dichlorooctane, C₈F₁₈, n-C₁₀F₂₂, n-C₁₀F₂₁H, n-C₈F₁₇C₂H₅, n-C₁₀F₂₁C₂H₅, n-C₈F₁₇CH═CH₂, n-C₄F₉CH═CH-n-C₄H₉, n-C₆F₁₃CH═CH-n-C₆F₁₃, n-C₈F₁₇CH═CH-n-C₄F₉, perfuoro-methyladamantane, perfuoro-dimethaladamantane, perfuoro-methyldecalin, (CF₃)₂CFOC₆F₁₃, (CF₃)₂CFO(CF₂)₄OCF(CF₃)₂, (CF₃)₂CFO(CF₂)₈OCF(CF₃)₂, perfuoro-i sopentyltetrahydropyran, perfuoro-butyltetrahydrofuran, perfuoro-N-methyldibutylamine, perfuoro-N-N-Diethylcyclohexyalmine, and perfuoro-tri-n-butylamine, or a combination thereof. In other Aspects of this Embodiment, the solvents may include or comprise any one or more of the solvents described in Jean G. Riess and Maurice LeBlanc, “Perfluoro Compounds as Blood Substitutes,” Angew. Chem., 17 (9)), 1978, pp. 621-700 and Camila Irene Castro and Juan Carlos Briceno, “Perfluorocarbon-Based Oxygen Carriers: Review of Products and Trials,” Artificial Organs, 34(8): 2010, pp. 622-634, each of which is incorporated by reference herein in its entirety for all purposes, or at least for the solvents and their methods of making and use.

Embodiment 40. The method of any one of Embodiments 1 to 39, wherein the near-infrared (NIR) photoactive direct treatment composition further comprises, an additive that enhances the solubility of the near-infrared dye.

Embodiment 41. The method of Embodiment 40, wherein the additive is a surfactant or alkali metal salt.

In certain Aspects of this Embodiment, the additive is an alkali halide, preferably sodium iodide, independently present at a level in a range from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the direct treatment composition. In other Aspects, the use of sodium iodide is specifically excluded.

In other independent Aspects of this Embodiment, the additive comprises a cationic surfactant, for comprising ammonium moiety, such as benzalkonium chloride. In other Aspects, the use of benzalkonium chloride is specifically excluded. In other independent Aspects of this Embodiment, the additive comprises an anionic or amphoteric surfactant. In still other Aspects of this Embodiment, the surfactant is independently present at a level in a range from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the direct treatment composition. In still other Aspects of this Embodiment, the surfactant is acceptable for use in human patients.

Embodiment 42. The method of any one of claims 1 to 41, wherein the near-infrared-(NIR) photoactive direct treatment composition is oxygenated before or during the irradiation. In preferred Aspects of this Embodiment, the direct treatment composition comprises dissolved oxygen at a level that exceeds that of the concentration of dissolved oxygen when in the presence of ambient atmospheric air. Additionally, or alternatively, the direct treatment composition comprises dissolved oxygen at a level within 50%, 55%, 60%, 65%, 70%, 75%, 80%, 85%, 90%, or 95% of the saturation limit of oxygen in the composition, when the composition is in the presence of pure oxygen.

Embodiment 43. A composition comprising a near-infrared dye that generates singlet oxygen when irradiated with near-infrared light in the presence of oxygen for use in any one of the methods for treating tissue set forth in Embodiments 1 to 42 or Aspects thereof or for treating any of the conditions disclosed herein. In certain Aspects of this Embodiment, the composition is a near-infrared (NIR) photoactive direct treatment as elsewhere described herein. In other independent Aspects of this Embodiment the near-infrared (NIR) photoactive direct treatment composition or the near-infrared dye includes any one or more of the features attributed to it in Embodiments 1 to 42, in any combination thereof.

Embodiment 44. A composition comprising a near-infrared dye that generates singlet oxygen when irradiated with near-infrared light in the presence of oxygen useful for use in the methods of Embodiments 1 to 42, wherein the composition comprises:

(a) a compound comprising a near-infrared (NIR) absorbing dye that generates singlet oxygen, when irradiated with near-infrared light in the presence of oxygen; and

(b) one or more of
(i) an optically transparent, biocompatible solvent
(ii) a biocompatible solvent having an oxygen solubility that is greater than the oxygen solubility in H₂O under comparable oxygen partial pressures, preferably a fluorinated or perfluorinated solvent; or
(iii) a biocompatible solvent comprising an additive that provides a solubility of the near-infrared (NIR) absorbing dye in that solvent that is higher than the solubility of the near-infrared (NIR) absorbing dye in the absence of the additive, preferably a surfactant or alkali metal salt, preferably independently present at a level in a range from 100 ppm to 0.1 wt %, from 0.1 w% to 0.5 wt %, from 0.5 wt % to 1 wt %, from 1 wt % to 1.5 wt %, from 1.5 wt % to 2 wt %, from 2 wt % to 3 wt %, from 3 wt % to 4 wt %, from 4 wt % to 5 wt %, from 5 wt % to 7.5 wt %, from 7.5 wt % to 10 wt %, from 10 wt % to 15 wt %, from 15 wt % to 20 wt %, from 20 wt %, to 25 wt %, from 25 wt % to 30 wt %, from 30 wt % to 40 wt %, from 40 wt % to 50 wt %, or a range defined by two or more of the foregoing ranges, relative to the total weight of the direct treatment composition;
(iv) a biocompatible, deuterated solvent, preferably D₂O;
(v) a biocompatible solvent comprising oxygen dissolved at a level that is higher than the equilibrium concentration of oxygen when exposed to ambient atmospheric air; or
(vi) a combination of two or more of (i) to (v).

In certain Aspects of this Embodiment, the composition is a near-infrared (NIR) photoactive direct treatment composition as elsewhere described herein. In other independent Aspects of this Embodiment, each of the near-infrared (NIR) absorbing dye, the biocompatible solvent, or the other descriptions attributed to this composition comprise aspects as attributed to the methods set forth herein.

Independent Aspects of this Embodiment, include those where the near-infrared light is defined in terms of any ranged defined herein, including at least one wavelength in a range of from 750 nm to 800 nm, from 800 nm to 850 nm, from 850 nm to 900 nm, from 900 nm to 950 nm, from 950 nm to 1000 nm, from 1000 nm to 1050 nm, from 1050 nm to 1100 nm, from 1100 nm to 1150 nm, from 1150 nm to 1200 nm, from 1200 nm to 1250 nm, from 1250 to 1300 nm, from 1300 to 1350 nm, from 1350 nm to 1400 nm, or in a range comprising two of more of these foregoing ranges

In independent Aspects of this Embodiment, the composition comprises D₂O. In independent Aspects of this Embodiment, the composition comprises a fluorinated or perfluorinated solvent as set forth elsewhere herein. In independent Aspects of this Embodiment, the composition comprises an additive that provides a solubility of the near-infrared (NIR) absorbing dye in H₂O that is higher than the solubility of the near-infrared (NIR) absorbing dye in the absence of the additive, the additive being of the kind and at levels as set forth elsewhere herein.

In independent Aspects of this Embodiment, the composition comprises oxygen at levels in excess of those associated with the composition being in contact with ambient atmospheric air. In preferred independent Aspects of this Embodiment, the direct treatment composition comprises dissolved oxygen at a level within 50%, 45%, 40%, 35%, 30%, 25%, 20%, 15%, 10%, or 5% of the saturation limit of oxygen in the composition, when the composition is in the presence of pure oxygen.

In independent Aspects of this Embodiment may be defined as a composition for treating any one or more of the conditions or targeted outcomes defined elsewhere herein.

Embodiment 45. The composition of Embodiment 43, further comprises a crosslinking compound. In Aspects of this Embodiment, the crosslinking compound may comprise a compound normally found in a sclera, such as a protein, polysaccharide, carbohydrate, glycosaminoglycan, proteoglycan, or combination thereof. In specific Aspects of this Embodiment, the crosslinking compound is or comprises collagen. In another specific Aspect of this Embodiment, the compound is or comprises glyceraldehyde.

### EXAMPLES

The following Examples are provided to illustrate some of the concepts described within this disclosure. While each Example is considered to provide specific individual embodiments of composition, methods of preparation and use, none of the Examples should be considered to limit the more general embodiments described herein.

In the following examples, efforts have been made to ensure accuracy with respect to numbers used (e.g. amounts, temperature, etc.) but some experimental error and deviation should be accounted for. Unless indicated otherwise, temperature is in degrees C., pressure is at or near atmospheric.

### Example 1: Materials and Methods

### Example 1.1. Singlet Oxygen Sensor Green Measurements

Singlet oxygen sensor green (SOSG) is a common reagent for measuring singlet oxygen generation in aqueous systems. The marker becomes more fluorescent as singlet oxygen is generated. All time measurements were done in triplicate (100 μL aliquots from an irradiated sample). A Molecular Devices Flexstation was used to read out the fluorescence measurements with the excitation/emission at 488/525 nm.

SOSG fluorescence turn on was initially measured using riboflavin as a point of comparison for the efficacy of ICG irradiations. Measurements used a dye concentration of 0.1 mg/mL and the irradiation the parameters (3 mW at 365 nm) used in the FDA approved procedure for keratoconus (corneal expansion). The mixture was stirred during irradiation.

Stocks of the ICG formulations (both H₂O and D₂O) were made to be 1 mg/mL ICG and 0.01% benzalkonium chloride (BAC). These were diluted to a dye concentration of 0.1 mg/mL for the SOSG tests with no adjustments for the BAC. The sample was irradiated with 810 nm light at 200 mW and stirred throughout the irradiation.

### Example 1.2. Young's Modulus Measurements

All Young's Modulus values were determined using an Instron equipped with screw side action tensile grips and a load cell rated for max 100 N. Rates of pulling were between 3 mm/minute and 5 mm/minute. The software calculated the Young's Modulus from the force vs. distance/time.

Bovine gelatin sheets were made by heating the gelatin in Dulbecco's buffer to 75° C. for 30 minutes in order to dissolve the dried gelatin and make a viscous solution. Neat TEOA and a concentrated stock solution of ICG in deionized water were added to make the final concentrations: bovine gelatin=25% wt, TEOA=90 mM, and ICG=1 mg/mL. This solution was pipetted hot into a plexiglass mold with a Teflon spacer to form a sheet of the following dimensions: 14 cm×6 cm×1 mm. It was stored overnight in a 4° C. refrigerator to solidify. Twelve 1 cm×6 cm strips were cut from the solidified gel for triplicate analysis of 4 conditions: no irradiation, 3 minute, 5 minute, and 10 minute irradiations. An 810 nm 200 mW LED (Thor Labs) was used for treatment of the gel strip. The LED with a collimator produced a 1 cm×1 cm irradiation area, which was used to treat 1 cm portions of the gelatin strip at a time. For example, an initial irradiation was applied for 3 minutes on 1 cm of the gel, then the gel was shifted 1 cm to a new, untreated portion. This was repeated until the whole strip received a total of 3 minutes of irradiation. Unfortunately, the 10 minute irradiation was ignored because it caused noticeable dehydration of the sample. All strips were tested on the Instron at a 5 mm/minute pull rate. The Young's Modulus values of the 3 minute and 5 minute irradiations were similar and compiled into one value for comparison to the no irradiation value. A 26% increase of the Young's Modulus was observed for the irradiated sample compared to the non-irradiated sample (FIG. 3).

The treatment was also tested on a more biologically relevant sample—unscalded porcine (pig) eyes (Sierra For Medical Science). Eyes were cut around the globe to make a 1 cm×5-6 cm strip of pig sclera for testing. Measurements were accumulated over several batches of eyes. Storage solutions before treatment (irradiation) contained 20% Dextran (MW ˜40-45k) in D₂O while post-treatment storage was Dulbecco's buffer with 20% Dextran (MW ˜40-45k). An ICG/BAC/D₂O soak solution was made to the concentration of 2 mg/mL ICG and 0.01% BAC. Strips for treatment were soaked in the solution for 0.5-2 hours. Irradiation was carried out similarly to the bovine gelatin strips—810 nm LED, 200 mW, and a 1 cm×1 cm irradiation zone. To ensure sufficient irradiation without sample dehydration, irradiation duration was kept at 5 minutes but performed twice—i.e. the sample was moved down 1 cm every 5 minutes and then the process was repeated—in order to give a total of 10 minutes of irradiation. Several methods were explored to ensure that the strips stayed hydrated including: placing them on moistened gauze pads, placing them into a shallow solution of D₂O, adding drops to the treated zone every 1-2 minutes, and placing them on wax paper. Despite these slight variations, the Young's Modulus values did not differ significantly (as was the case with the gelatin samples), and therefore these values were aggregated for quantitative analysis. All scleral strips were loaded onto the Instron with 180 grit sandpaper where the grips held the sample and pulled at a rate of 3 mm/minute. The Young's Modulus values were calculated by the software and compiled to compare untreated vs. treated strips. A 21% increase of the Young's Modulus was observed for the irradiated sample compared to the non-irradiated sample (FIG. 3).

Eye Expansion Test—Whole Eye Treatment

Enucleated young rabbit eyes were provided from collaborators at University of California, San Francisco (UCSF). These were shipped overnight and used within 1-4 days. Upon arrival, they were stored in Dulbecco's buffer in a 4° C. refrigerator until use. They were trimmed of fat and muscle to provide a smooth surface for easy identification of changes to the size. All irradiations used an 810 nm LED (Thor) set to a power of ˜200 mW.

Untreated eyes received no further modification and were stored in Dulbecco's buffer in the refrigerator until use. Eyes to be treated had their epithelial layer on the cornea removed. An ethanol-soaked Kim wipe was applied to the cornea for two minutes and then a scalpel blade was used to scrape the layer off. Removal was confirmed using a fluorescein strip and long UV light. The prepared eye was incubated in a soaking solution of 2 mg/mL ICG and 0.01% BAC in D₂O for 0.5 to 2 hours with gentle shaking. The eye was placed cornea up on a damp (D₂O) gauze pad and then placed on a 10 mL beaker. The LED was placed above the eye to irradiate down for 10 minutes with 5-15 drops of either the soaking solution or D₂O being applied every 2 minutes to keep the sample hydrated. Once completed, the irradiation process was repeated for the reverse side of the eye (by the retina), then the eye on its side (half cornea & sclera), and finally on the reverse side of the sideways eye. This was a total of four 10 minute irradiations aimed to treat the whole eye.

The eye expansion setup followed the protocol outlined below. The eyes were placed on a stand and 30 gauge needle was inserted from underneath. The chamber was filled with Dulbecco's buffer and two different antibiotics mixtures—an ophthalmic solution of Trimethoprim Sulfate and Polymyxin B Sulfate and an ophthalmic ointment of Neomycin and Polmyxin B Sulfates and Bacitracin Zinc. A low pressure (˜22 mm Hg) was applied for one hour to equilibrate the eyes prior to applying a higher pressure (˜85 mm Hg) for the experiment. This is done by raising an IV bag to a predetermined height above the eye chamber. The higher pressure was applied from 12-36 hours (or until rupture) depending on the experiment. Two photos were taken every 15 minutes during both the low and high-pressure applications. The second photo was always used in analysis and video generation.

Image J was used to calculate the scleral area for comparison. The hand drawn function was used to outline the sclera only. These measurements were done until subsequent traces produced an area that did not fluctuate by more than 0.01 inches2. This was corroborated for consistency by using the “threshold” function of Image J, which showed similar calculated values. The hand drawn approach was used for ease of subtracting out the undesired corneal area. Measured scleral areas where then converted to percent change from the initial time point (when the higher pressure was first applied). These values were averaged and standard errors calculated for comparison.

Eye Expansion Test—Split Eye Treatment

The split eye expansion tests followed the whole eye test protocol except during the incubation and irradiation steps. All other methods were similar including the analysis.

To incubate only half the eye, a small plastic stand was used for the eye to sit on while in the soaking solution. Every 5-10 minutes, the eye was picked up and agitated to ensure dye made it to the portion of the sclera that was touching the incubation stand. Extreme care was taken to place the eye back on the stand in the proper orientation to keep a distinct line between the treated and untreated portion. This was done over the course of 30-60 minutes.

Irradiation was performed twice—once with the center of the LED over the cornea and the second time over the majority of the sclera, all on the ICG treated side. Here, only D₂O was dropped onto the eye to keep it from drying out. A total of two 10 minute irradiations were done to the treated side.

Eye expansion setup was consistent with the whole eye test except the arrangement of the eye on the stand. Here, care was taken to line the eye up so the different halves (treated and untreated) were equally represented in the camera photograph. An asymmetric expansion was seen in the eyes and was captured through the area analysis described above.

### Example 2. Results and Discussions

Here is described a formulation consisting of three components—a NIR absorbing dye that generates singlet oxygen, an additive to aid in solubility of the dye, and a solvent to enhance the availability of singlet oxygen for crosslinking. There are many permutations on this set of three components that could be used for our described purpose, some of which omit a component entirely. However, for these experiments, the focus was on a formulation expected to generate a significant amount of singlet oxygen. Specifically, the FDA approved dye indocyanine green (ICG) was used with the cationic surfactant benzalkonium chloride (BAC) or sodium iodide, and deuterated water (D₂O). This formulation demonstrated successful reinforcement of the sclera upon irradiation, marking a starting point for future formulations.

ICG is a NIR-absorbing heptamethine dye that has the potential of generating singlet oxygen upon irradiation and has a λₘₐₓ ranging from 780-810 nm depending on the context. Other derivatives exist that have similar structures but contain halogens, and these are shown to increase singlet oxygen generation efficiency (Example: IR-820 or “New Indocyanine Green”). These would also be effective chromophores; however, they are not FDA approved and thus were not selected for our initial formulation. BAC along with other cationic surfactants (alkyl ammonium salts, polyoxyethylene alkyl amines, amide linked alkyl amines, alkyl imidazolines, etc.) aid in the solubility of the dye in aqueous media. An enhanced solubility of ICG (higher concentrations going to complete dissolution) was observed when these additives where used. Sodium iodide is also known to increase ICG solubility, but greater increases in solubility were observed with BAC, therefore BAC was selected for our initial formulations. Other halogen salts could also be effective additives to aid in ICG solubility. Finally, other biological macromolecules (lipids, proteins, peptides, etc.) could be used to alter the ICG concentration as seen with bovine serum albumin (BSA). The standard formulation used in these studies consisted of 0.05 to 5.0 mg/mL ICG and 0.01% to 0.001% BAC in solution. D₂O was selected as the solvent to increase the lifetime of singlet oxygen and therefore the crosslinking capability of the species. Other deuterated (ethanol, dimethyl sulfoxide, etc) are expected to produce a similar increase in singlet oxygen lifetimes. D₂O exposure to the eye is considered safe due to the short lifetime of exposure, the small amount used, and the lack of adverse effects that have been observed in other studies. Fluorinated solvents (perfluorohexane, perfluorotripentylamine, etc.) are also of interest due to their ability to dissolve more oxygen and their robust inertness. Delivering more oxygen to the area would enhance scleral cross-linking.

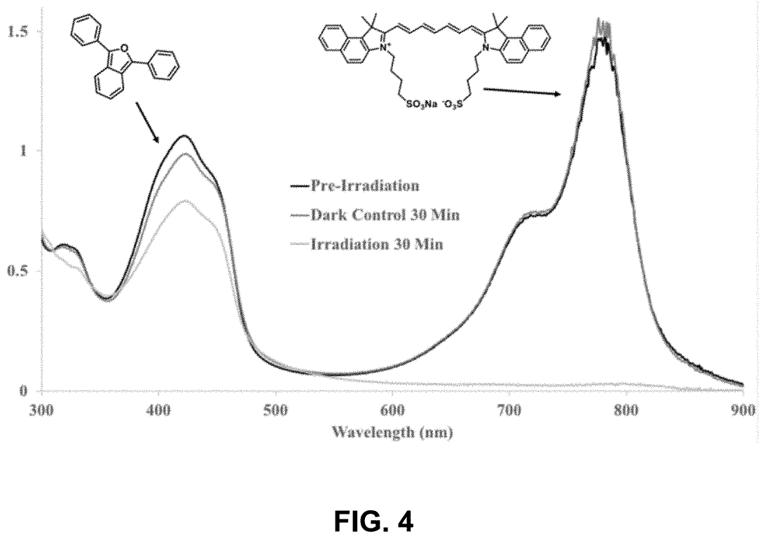

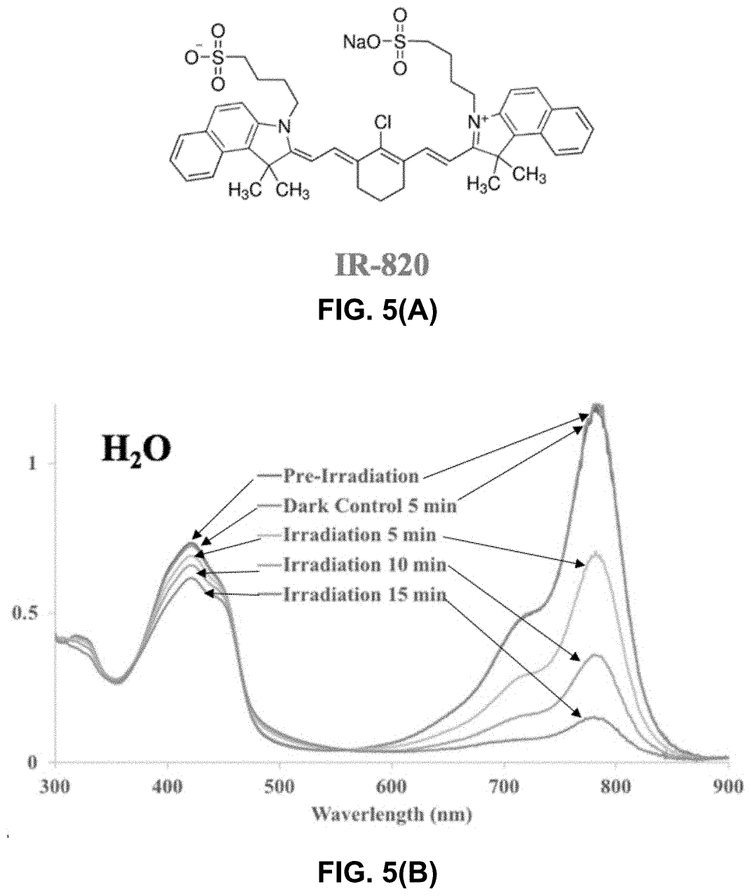

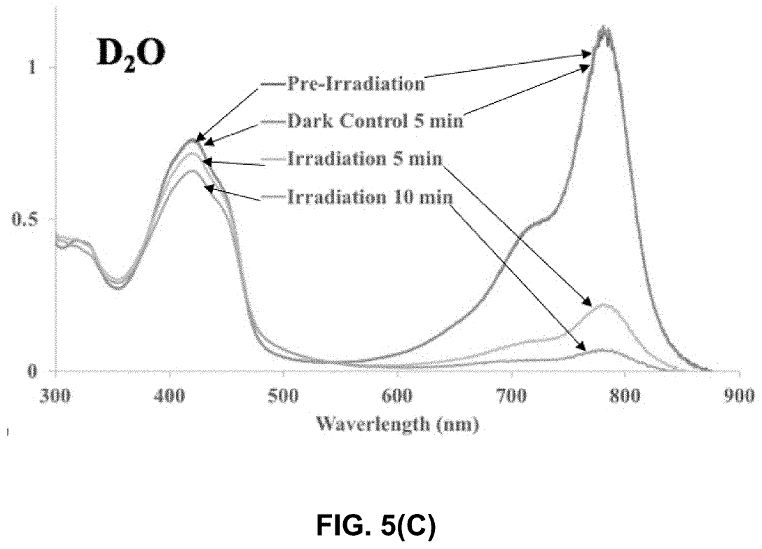

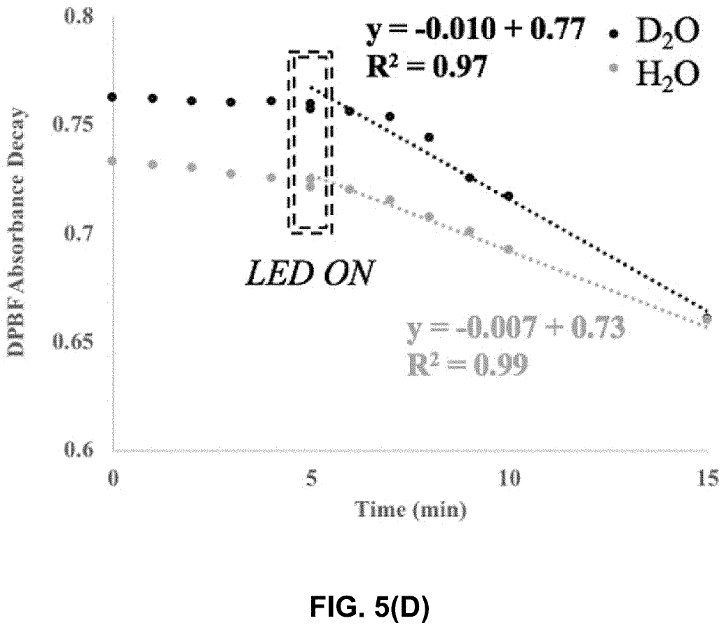

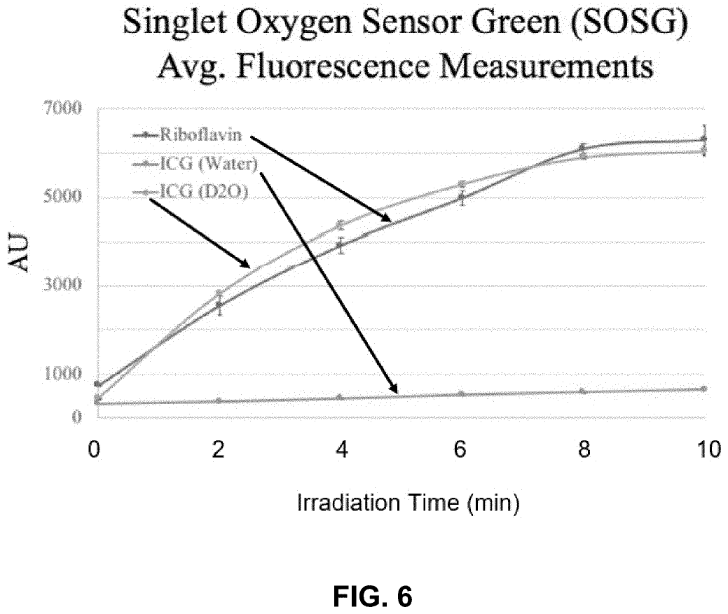

To evaluate the efficiency of singlet oxygen generation by ICG and an analog (IR-820) in vitro, the singlet oxygen generation was monitored. 1,3-diphenylisobenzofuran (DPBF), a compound that decays in the presence of singlet oxygen, and singlet oxygen sensor green (SOSG), a dye that increases fluorescence in the presence of singlet oxygen, were used as markers to indicate singlet oxygen generation by ICG and IR-820. For DPBF analysis, the activity of ICG and IR820 was observed in H₂O or D₂O. In all tests, singlet oxygen generation was observed (FIGS. 4 and 5) and an increase was seen in the deuterated solvent (FIG. 5). For SOSG analysis, the fluorescence turn-on in H₂O and D₂O was compared to that observed when riboflavin—an FDA approved dye used in conjunction with UV light to treat keratoconus (corneal expansion)—was usedIn all cases, an increase in fluorescence emission at 525 nm was observed (using a 488nm excitation). However, as seen in FIG. 6, only in the presence of D₂O was ICG singlet oxygen generation efficient enough to compete with the turn-on observed in the presence of riboflavin.

To transfer these chemical results to a material system, the change in the stiffness of collagen-containing samples was measured, as collagen crosslinking has been shown to make materials stiffer and less resistance to change. Specifically, the Young's modulus was measured, which was expected to increase following crosslinking as more force would be required to stretch the material.

The ability of ICG to crosslink collagen was initially tested by impregnating a bovine gelatin sheet with ICG (1 mg/mL), triethanolamine (TEOA=90 mM), and Dulbecco's buffer. Twelve strips (1 cm×6 cm) were cut from the sheet and triplicate measurements were made for the following conditions: untreated, 3 minute irradiation, 5 minute irradiation, and 10 minute irradiation. The 10-minute condition gave inconclusive results due to dehydration of the gel. An Instron universal tester was used to measure the Young's Modulus. Measurements from the 3-minute and 5-minute irradiations produced similar values and were combined and compared to the untreated sample. A change of 26% was observed between the treated and untreated samples (FIG. 3).

Since a significant change was observed for the bovine gelatin, the treatment formulation was tested on unscalded pig sclera to see if a similar change could be induced. Pig sclera is inherently a tougher material and previous work has shown that only a minimal change is observed in the Young's Modulus when treated with UV light and riboflavin. Again, strips of ˜1 cm×5-6 cm of unscladed porcine sclera (Sierra For Medical Science) were cut and subjected them to an ICG/BAC/D₂O formulation and irradiation. Several variations on the experiment were explored in an attempt to reduce sample dehydration, including soaking in a Dextran (MW ˜40-45k) solution. Aggregating these data and comparing treated and untreated sclera yielded an increase of 21% in the Young's Modulus of the material (FIG. 3), close to that observed with the riboflavin treatment. These results gave us confidence in our formulation and treatment to pursue an eye expansion model test.

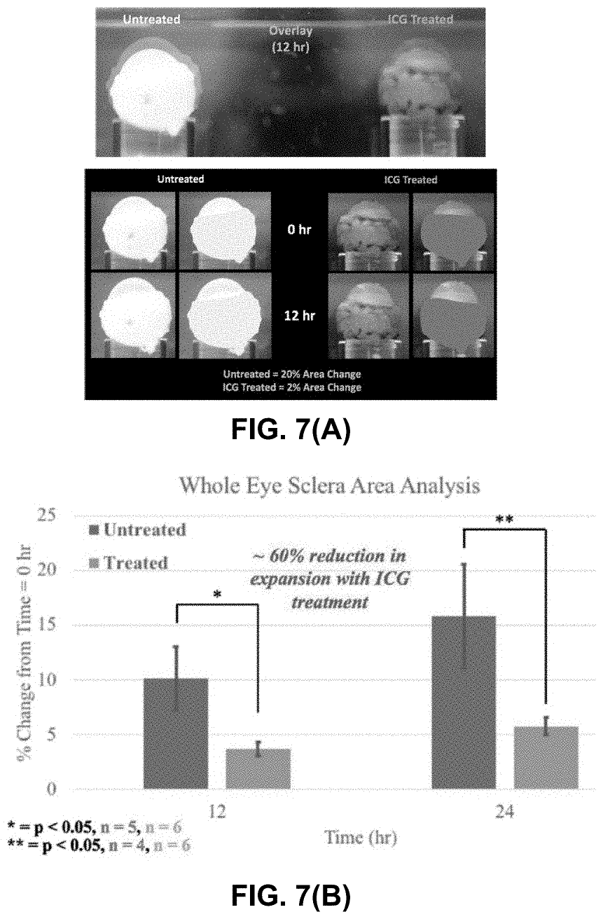

Eye expansion tests were previously used to demonstrate the ability of Eosin Y to crosslink the cornea and sclera. To evaluate the ability of ICG to crosslink the sclera, a similar set of eye expansion tests was performed. In the experimental setup, the untreated eye was not modified and used as received. The treated eye had the epithelial layer of the cornea removed and was subsequently incubated in an ICG/BAC/D₂O solution for 0.5-2 hours. The eye was irradiated on four sides for 10 min each with drops of the soaking solution or D₂O being applied every 2 minutes to keep it hydrated. Both eyes were then mounted in a chamber containing Dulbecco's buffer and subjected to intraocular pressure applied through a thin needle connected to a raised water reservoir. Images were captured every 15 minutes for 12-36 hours while the eyes were subjected to ˜85 mm Hg pressure. Interestingly, in all treated cases the cornea had a layer of ICG peel back during the expansion, which resulted in a large outward protrusion of the cornea. The sclera, however, consistently held its shape much better when treated with the ICG formulation. To quantify this observation, the area of the sclera was calculated using Image J for the initial and 12-hour time points (FIGS. 7(A-B)). Expansion from the initial time point was calculated and compared as a percent. Aggregating these data resulted in ˜60% reduction of expansion of the treated vs untreated eyes at the 12-hour time point (FIG. 7(B)).

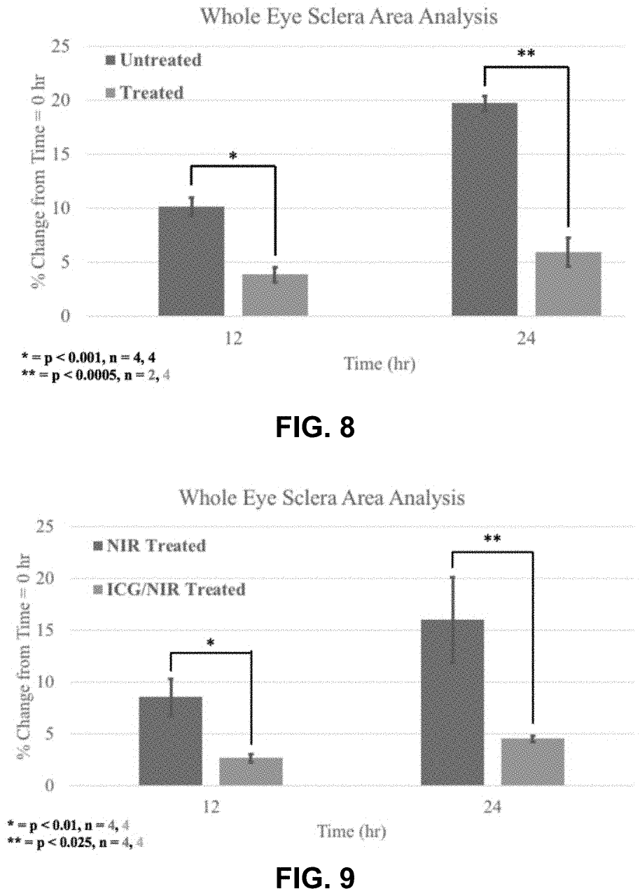

These experiments were repeated using sodium iodide (NaI) as the additive to aid in dissolving ICG instead of benzalkonium chloride (BAC). As shown in FIG. 8, time points at both 12-hours and 24-hours showed a reduction of ˜62% and —70%, respectively, in expansion between the treated and untreated portions of the eye.

These experiments were repeated using sodium iodide (NaI) as the additive and D₂O and treating the control eye with NIR light. As shown in FIG. 9, the time points at 12-hours and 24-hours showed a reduction of ˜69% and ˜72%, respectively, in expansion between the fully treated eyes and NIR light only treated eyes. This showed that NIR light was not enough to induce expansion reduction and that ICG was necessary in the treatment.

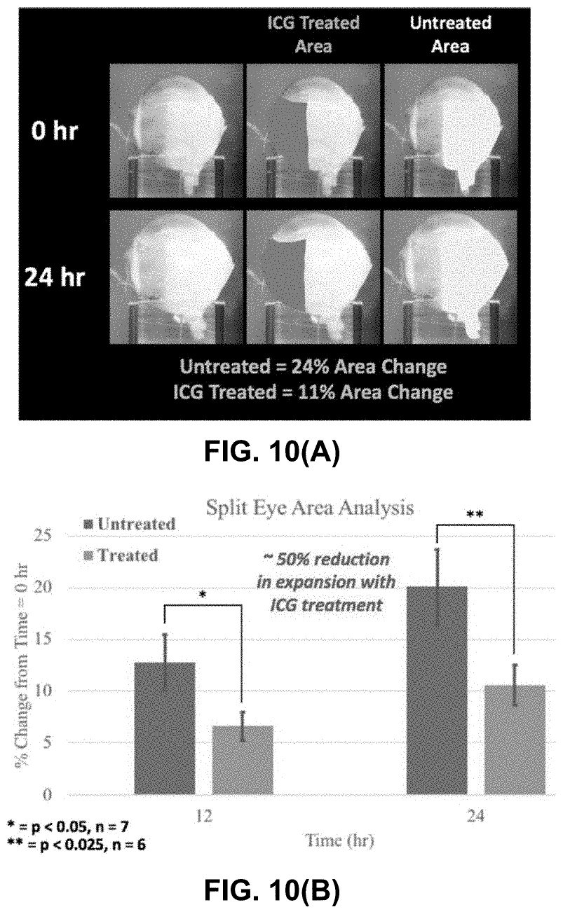

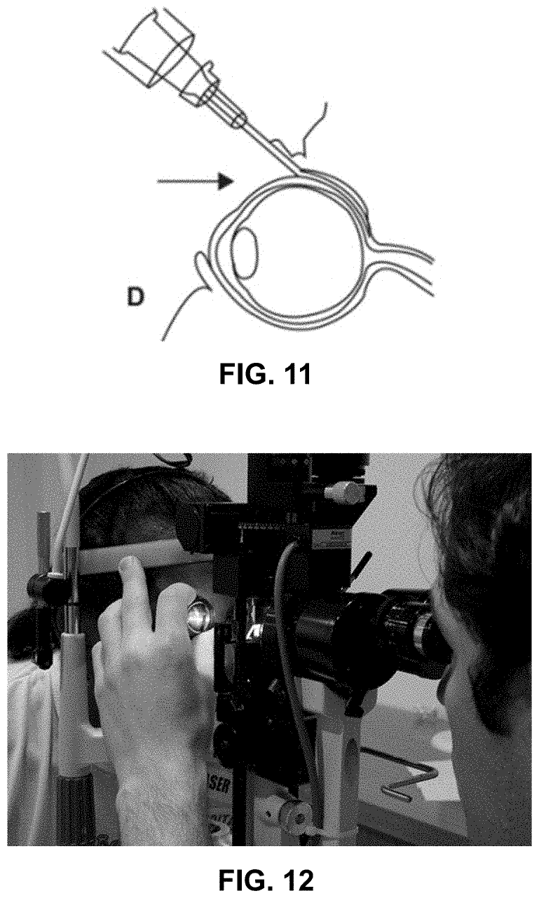

In an effort to further validate these results with regards to an internal standard, a second assay was developed. In this assay, only half of the eye received the treatment while the other half was left untreated, giving a “split” eye. This was accomplished by setting the eye on a stand and gently dunking it up and down every 5-10 minutes for a total of 30-60 minutes. Otherwise, the same eye expansion setup and data analysis was used as was used in the whole eye treatment test. By incorporating both the treated and untreated components into the same eye, the split eye approach reduced biological variability and allowed an asymmetric expansion in a single eye, robustly demonstrating the utility of the treatment. FIG. 10(A) clearly demonstrates significant expansion of the untreated side relative to the treated side. Quantifying this change, an ˜50% reduction in expansion of the treated side was observed compared to the untreated side at both the 12-hour and 24-hour time points (FIG. 10(B)).

### Example 3: Prophetic

A patient with progressive high myopia is administered an ICG formulation delivered by posterior subtenon's injection. Alternatively, the ICG formulation can be injected into the suprachoroidal space, intravenously, or by retrobulbar injection. Following the injection by 30-60 minutes, allowing adequate time for the ICG formulation to diffuse into the posterior sclera, the patient is seated at the 810 nm laser (for example, the The OcuLight® SL and OcuLight SLx 810 nm solid state laser by Iridex, Inc, Mountain View, Calif.). A contact lens is place on the cornea under topical anesthesia. Next, laser energy is directed at the posterior pole region where the posterior sclera is treated. Because the IR light is only partially absorbed as it traverses through the ocular media, retina, and choroid, it is transmitted to the sclera where it activates the ICG formulation. Resulting singlet oxygen generation effects scleral crosslinking and increases scleral modulus. Both the injection of the ICG formulation and subsequent irradiation can be directed at other regions of the sclera if indicated.

Post-operatively, the patient is monitored for changes in axial length and posterior scleral contour. If further changes occur indicating myopic progression, the ICG formulation injection and 810 nm irradiation can be repeated.

### Example 4: Prophetic

A patient with deep bacterial keratitis receives topical ICG formulation. After waiting adequate time for diffusion of the ICG formulation into the deep cornea, the patient is treated with 810 nm laser irradiation directed at the infected region to activate the ICG formulation. Resulting generation of singlet oxygen kills the infectious agent(s) in the cornea.

### Example 5: Prophetic

A patient with choroidal melanoma in the posterior pole is administered the ICG formulation by direct injection into the tumor or by intravenous administration. Next, the 810 nm laser is used to irradiate the tumor. Generation of singlet oxygen effects killing of melanoma cells.

### Example 6: Experiments to the Use of Dyes where Y is a Cationic Heteroaryl Moiety

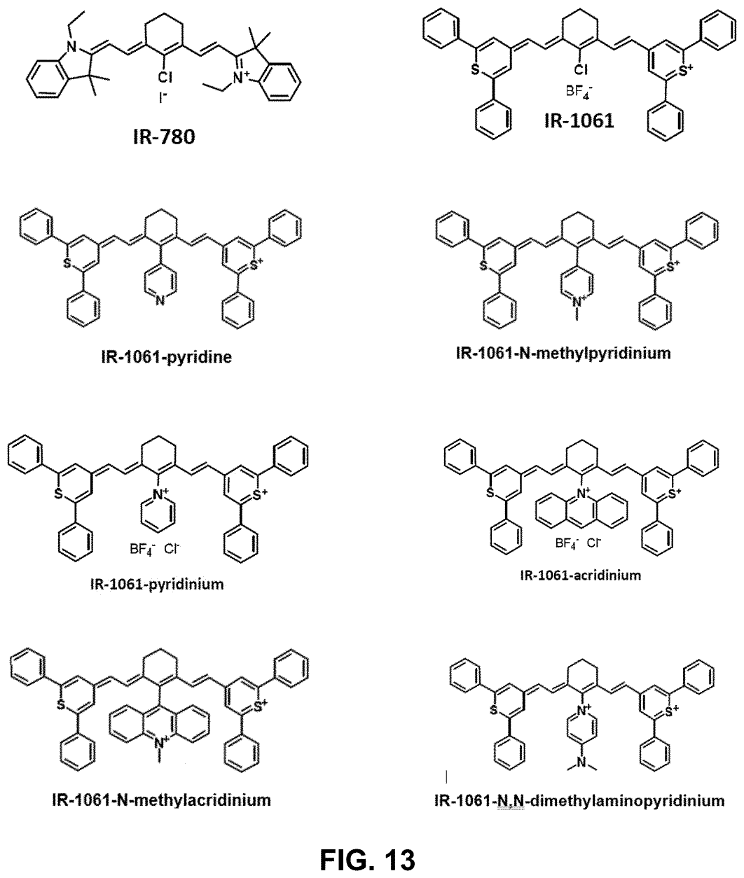

Various dyes where Y was a cationic heteroaryl moiety were also evaluated for their ability to generate singlet oxygen under conditions comparable to those described above (see FIG. 13).

### Example 6.1: IR-1061-Pyridinium Synthesis

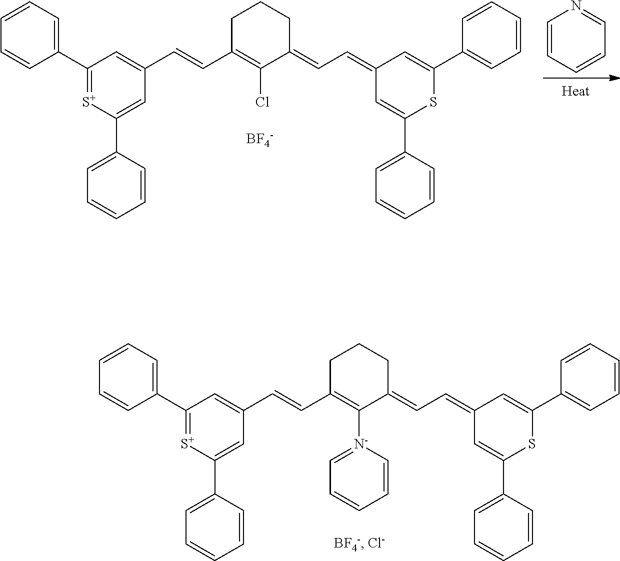

A 10 mL flame-dried round-bottom flask was charged with a stir bar and 25 mgs of IR-1061, to which 1 mL of dry pyridine was added. The reaction was heated to boiling under argon and removed from heat once the solution transitioned from dark red to greenish-brown. At this point the compound was purified by silica gel chromatography using a 0-5% MeOH in dichloromethane (DCM) gradient. The product could not be cleanly purified due to decomposition during purification. All other attempted purification conditions gave the same result. Due to low solubility and contaminating species, an interpretable NMR was never obtained, however, a reasonably clean mass spectrometry trace was obtained. MS (MALDI-TOF): (m/z) calculated for C₄₉H₃₈NS₂+: 704.24 (m-1). Observed 704.056 (m-1, presumably due to loss of a proton to alleviate the dual positive charge).

### Example 6.2: IR-1061-C-Bonded-Pyridinium Synthesis

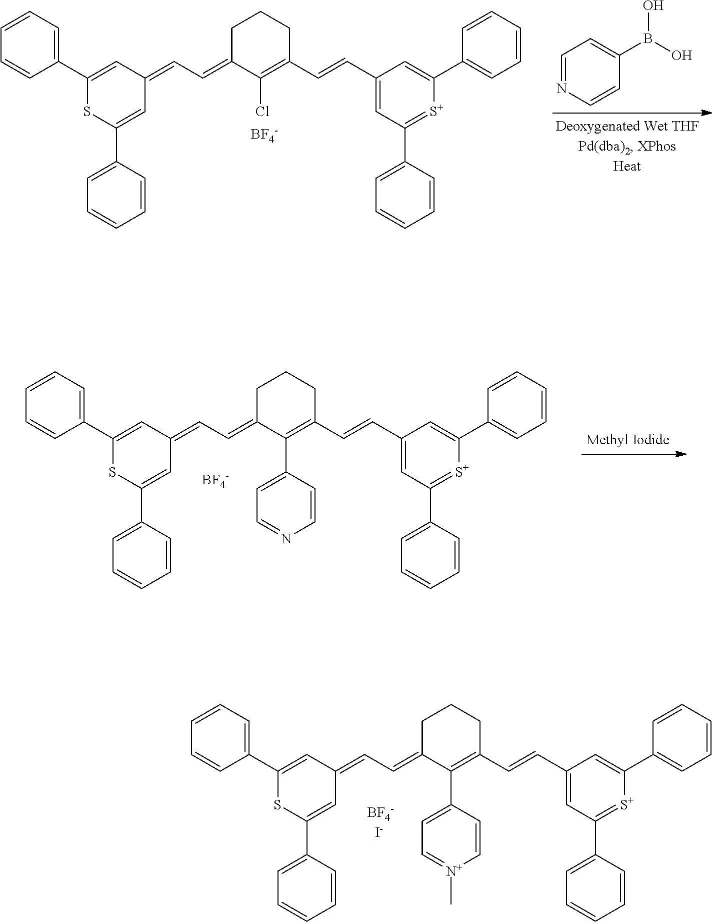

### Example 6.3: IR-1061-Acridinium Synthesis

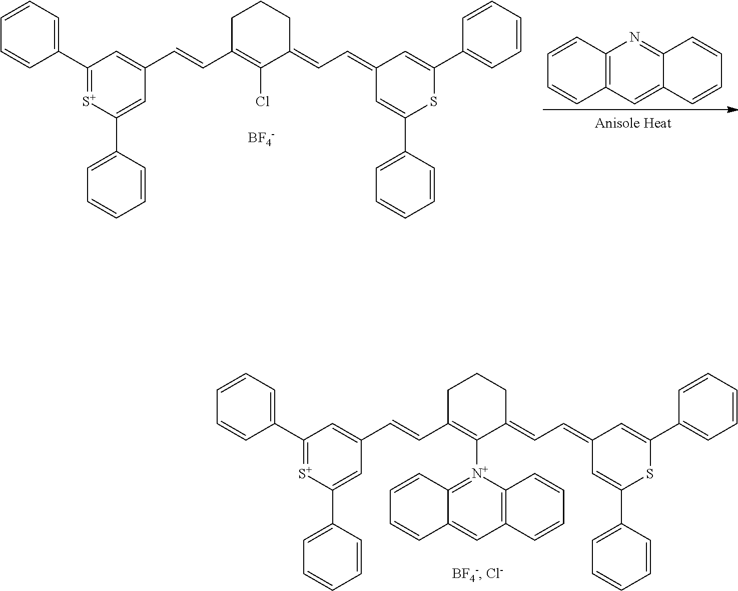

A 10 ml round-bottom flask was charged with a stir bar, 25 mgs of IR-1061 (1 equivalent), and 60 mgs of acridine (10 equivalents). Two milliliters of anisole were added, and the reaction was heated to boiling under argon. It was removed from heat once the color change from red to a yellow hued brown. Further heating led to formation of a green decomposition product. The reaction mixture was loaded onto a silica plug, and after eluting the anisole and any nonpolar compounds with DCM, a polar fraction containing the dye of interest was eluted with 5% MeOH in DCM. After pumping down, this polar fraction was resuspended in DCM and loaded onto a silica gel column, which was eluted with a 0-2% MeOH in DCM gradient. 20 mgs of pure product were collected as a brown-yellow compound, giving a percent yield of 64%. ¹H-NMR: 7.75 (m, 2H), 7.68-7.60 (m, 12H), 7.52-7.42 (m, 14 H), 6.99 (t, 2H) 6.91 (d, 2H), 6.65 (t, 2H), 6.59 (d, 2H), 6.28 (m, 1H), 2.84 (t, 2H), 2.56 (t, 2H), 1.95 (m, 2H) MS (ESI-TOF): (m/z) calculated for C₅₇H₄₂NS₂+: 804.275 (m-1). Observed 804.277 (m-1, due to loss of a proton to alleviate the dual positive charge).

### Example 6.4: IR-1061-Acridinium BArF Synthesis

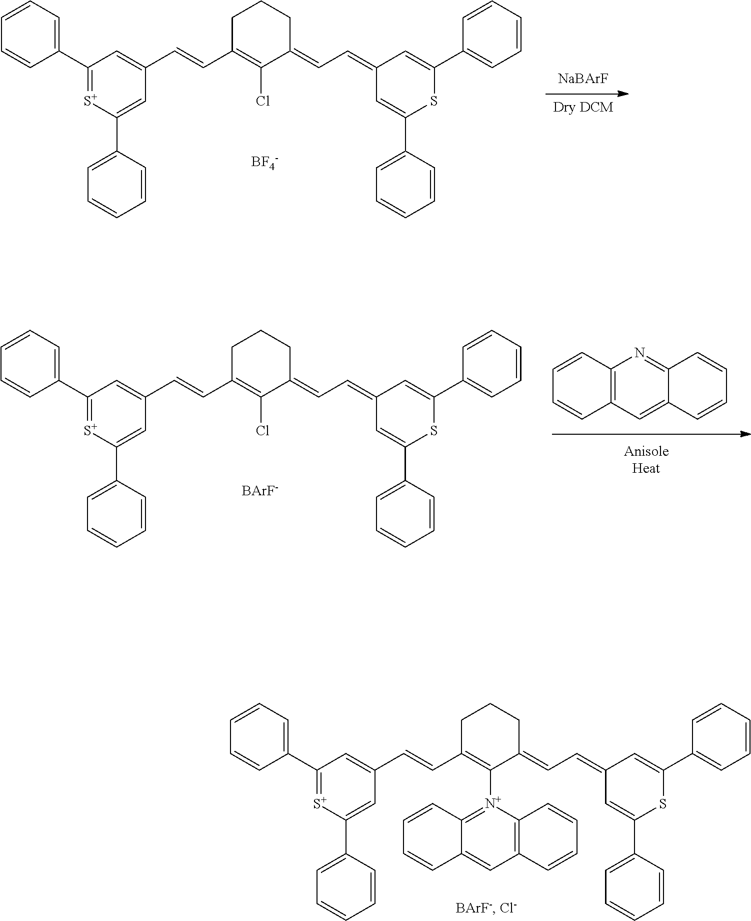

IR-1061 BarF⁻ was synthesized and purified in the manner previously described. Following isolation, 25 mgs of IR-1061 BArF (1 equivalent) was added to a 10 ml round-bottom flask along with a stir bar and 29.3 mgs of acridine (10 equivalents). Two milliliters of anisole were added, and the reaction was heated under argon. It was removed from heat once the color change from red to a yellow hued brown. Further heating led to formation of a green decomposition product. The reaction mixture was taken up in DCM and extracted three times with water and once with brine. The DCM fraction was pumped to near dryness, then resuspended in DCM and loaded onto a silica gel column. The product was eluted with a 0-1% MeOH in DCM gradient. 12 mgs of pure product were collected as a brown-yellow compound, giving a percent yield of 43%. NMR and mass spec data matched that of IR-1061-acridinium tetrafluoroborate, although significant peak broadening was observed on the MALDI with the BArF counterion. ¹H-NMR: 7.75 (m, 2H), 7.68-7.60 (m, 12H), 7.52-7.42 (m, 14 H), 6.99 (t, 2H) 6.91 (d, 2H), 6.65 (t, 2H), 6.59 (d, 2H), 6.28 (m, 1H), 2.84 (t, 2H), 2.56 (t, 2H), 1.95 (m, 2H) MS (MALDI): (m/z) calculated for C₅₇H₄₂NS₂+: 804.275 (m-1). Observed 804.5 (m-1, due to loss of a proton to alleviate the dual positive charge.

### Example 6.5: Results

The thiopyrilium dye IR-1061 was chosen for initial experiments due to its NIR absorption spectrum.

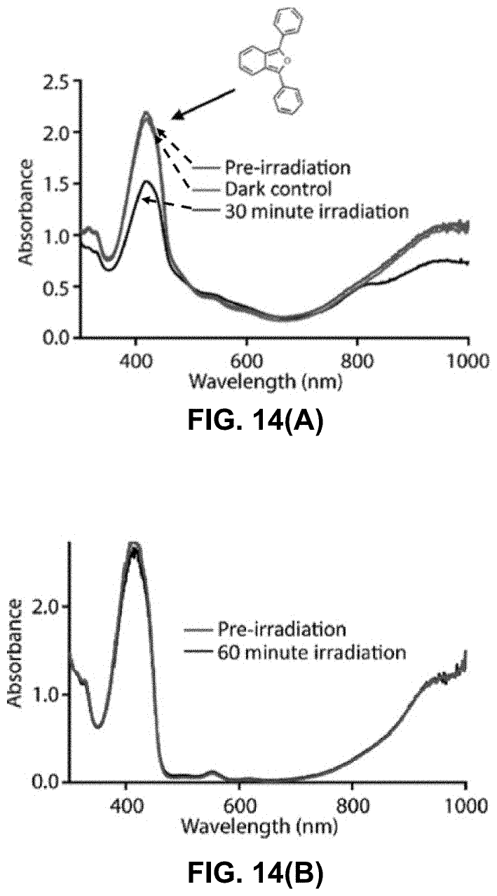

Initial work was done using a set of IR-1061 derivatives: cited herein as IR-1061-pyridine and IR-1061-pyridinium. After 30 minutes of irradiation using a 1 W cm⁻² 980 nm laser, a significant decrease in the DPBF signal was observed relative to a dark control, suggesting that singlet oxygen sensitization was occurring (FIG. 14(A)). In comparison, the parent dye, IR-1061, was irradiated under the same conditions and no significant changes in DPBF signal were observed (FIG. 14(B)). This suggested that the designed dye had novel reactivity at previously inaccessible wavelengths, but further characterization was required.

Unfortunately, IR-1061-pyridinium was unatable in the presence of water. In an attempt to overcome the susceptibility of IR-1061-pyridinium to hydration, the pyridine charge-transfer partner was exchanged for acridine, which was expected to shield the dye from water addition due to its additional bulk.

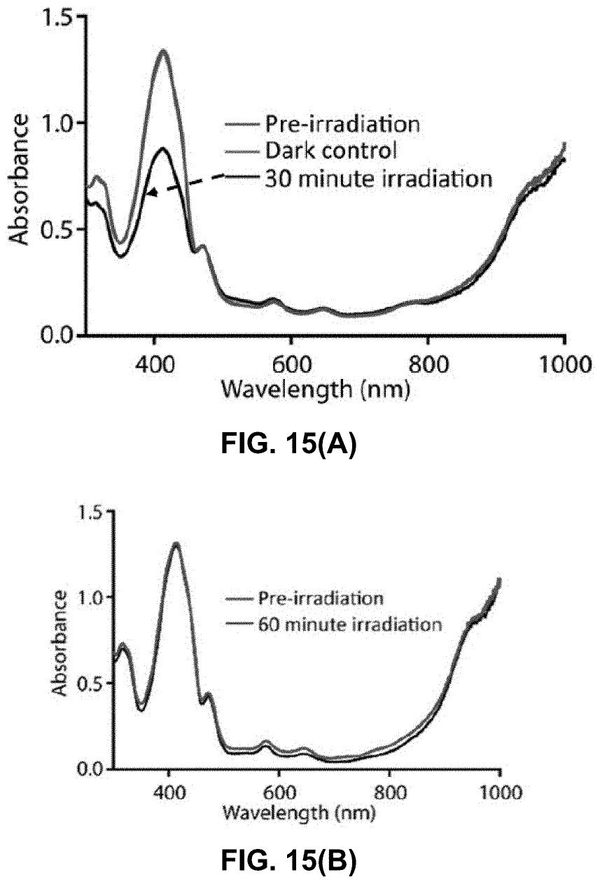

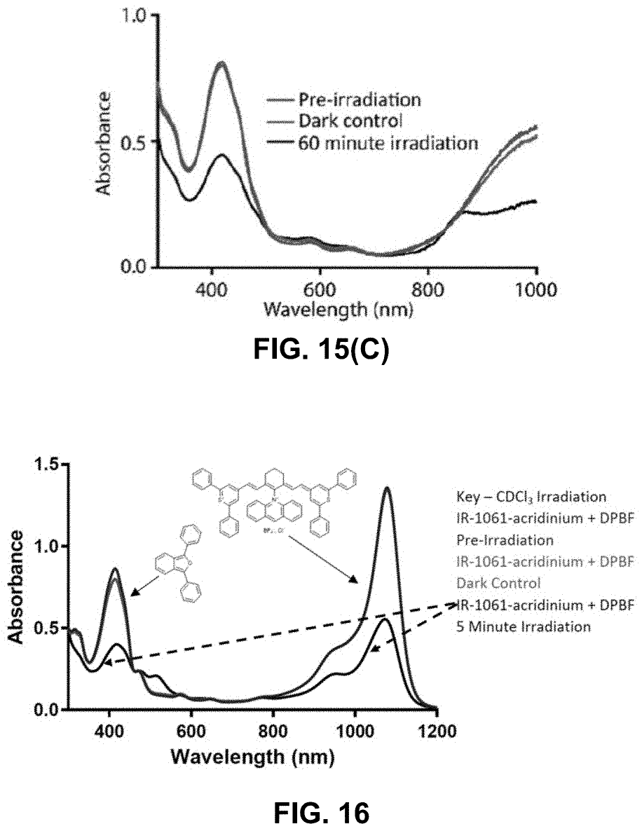

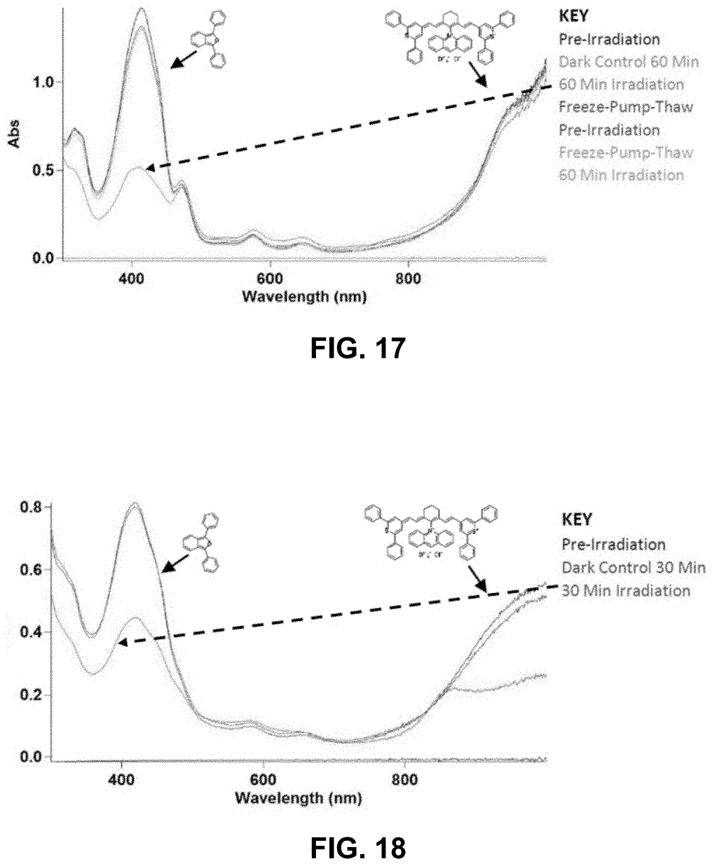

Like IR-1061-pyridinium, IR-1061-acridinium was irradiated in CDCl₃ in the presence of DPBF using a 1 W cm⁻² 980 nm laser (FIG. 15(A)). As a comparison, a similar irradiation was carried out using a 1.8 W cm⁻² 1064 nm laser (FIG. 16). The collective effect of irradiation wavelength and conditions are seen in FIGS. 15(A-C), and FIGS. 16-18.

To confirm that the process was singlet oxygen-mediated, irradiation of a freeze-pump-thawed sample—devoid of oxygen—was carried out. This irradiation produced no change in DPBF signal (FIG. 15(B)), consistent with the idea that the process was singlet oxygen mediated. To evaluate the scope of the reaction, the dye was irradiated in an array of deuterated solvents. In nonpolar solvents, such as toluene, no singlet oxygen generation was observed. In polar aprotic solvents, such as DMSO, irradiation of the dye led to decreased DPBF degradation as well as photobleaching. In water, DPBF degradation rates comparable to those seen in chloroform were accompanied by high levels of photobleaching (FIG. 15(C)). Strong acids and bases both led to dye instability and bleaching prior to irradiation. Exchanging the counterion from BF₄⁻ to BarF⁻ resulted in an increase in solubility, but also a marked decrease in photosensitization capability.

Each patent, patent application, and publication cited or described in this document is hereby incorporated herein by reference, each in its entirety, for all purposes, or at least for the purposes or in the context where it was cited.

1. A method of altering a mechanical and/or chemical property of a tissue, the method comprising irradiating a near-infrared-(NIR) photoactive direct treatment composition with near-infrared light in the presence of oxygen;
wherein the near-infrared (NIR) photoactive direct treatment composition comprises a near-infrared absorbing dye having a structure of: 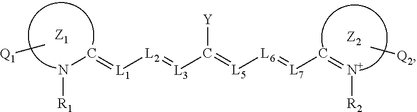
or a rotational or conformational isomer or a salt thereof;
wherein
L₁, L₂, L₃, L₅, L₆, and L₇ are substituted or unsubstituted methines, wherein the optional substituents are independently C₁₋₆ alkyl or C₂₋₆ alkenyl; or L₁ and L₃, or L₃ and L₅, or L₅ and L₇ are linked with a C₂₋₄ alkylene or C₂₋₄ alkenylene substituent to form a 5- to 7-membered ring;
each of Z₁ and Z₂ is independently a five- or six-membered nitrogen-containing heterocyclic ring, optionally fused to another aryl or heteroaryl ring;
each of Q₁ and Q₂ is independently H or a substituent positioned on the five- or six-membered nitrogen-containing heterocyclic ring and/or the optionally fused aryl or heteroaryl ring, each optional substituent comprising an optionally substituted C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo, nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R₁₀ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof, or a —(C₀₋₁₂alkyl)borate;
R₁ and R₂ is independently C₁₋₁₂ alkyl, —[CH₂—CH₂—O—]₁₋₆R¹⁰, —(C₀₋₁₂alkyl)amino acid residue, or a 5- or 6-member ringed aryl or heteroaryl, each of which may be optionally substituted with one or more —(C₀₋₁₂alkyl)(SO₃)—R¹⁰ or a salt thereof, —(C₀₋₁₂alkyl)(SO4)—R¹⁰ or a salt thereof, —(C₀₋₁₂alkyl)(PO₄)—R¹⁰ or a salt thereof, —(C₀₋₁₂alkyl)OR¹⁰, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂, —(C₀₋₁₂alkyl)N(R¹⁰)₂ or a salt thereof or —(C₀₋₁₂alkyl)borate or borate ester;
R¹⁰ is independently H or C₁₋₆ alkyl; and
Y is H, or an optionally substituted amine, optionally substituted alkyl, optionally substituted alkoxy, optionally substituted aryl, optionally substituted heteroaryl, optionally substituted aryloxy, optionally substituted heteroaryloxy, halogen, or optionally substituted cationic heteroaryl moiety;
that generates singlet oxygen when irradiated with near-infrared light in the presence of oxygen;
wherein the near-infrared (NIR) photoactive direct treatment composition is adjacent to (contacts) or has permeated the tissue; and
wherein the irradiating results in a change in the mechanical and/or chemical property of a tissue.

2. The method of claim 1, wherein the mechanical and/or chemical property is tensile strength, compression strength, flexural strength, modulus, elongation, or toughness of the tissue.

3. The method of claim 1, wherein the tissue is an ocular tissue.

4. The method of claim 3, wherein the ocular tissue includes at least a portion of a cornea, a sclera, or a lamina cribrosa.

5. (canceled)

6. The method of claim 1, further comprising administering the near-infrared (NIR) photoactive direct treatment composition to the tissue of the a patient, either topically or by injection.

7. (canceled)

8. The method of claim 1, wherein:
(a) the five- or six-membered nitrogen-containing heterocyclic rings of Z₁ and Z₂ independently comprise a pyrrole ring, imidazole ring, isothiazole ring, isoxazole ring, oxadiazole ring, oxazole ring, pyrazole ring, pyrimidyl, thiazole ring, selenazole ring, thiadiazole ring, triazole ring, or a pyridine ring;
(b) the five- or six-membered nitrogen-containing heterocyclic rings of Z₁ and Z₂ are independently fused to a phenyl, naphthyl, pyridinyl, quinolinyl, quinoxalinyl, N-alkyl-benzoindolenine, dibenzofuranyl, or dibenzothiophenyl; and/or
(c) Z₁ and Z₂ independently comprise a benzimidazole ring, benzindole ring, benzoindolenine ring, benzoxazole ring, benzothiazole ring, furopyrrole ring, imidazole ring, imidazoquinoxaline ring, indolenine ring, indolizine ring, isoxazole ring, naphthimidazole ring, naphthothiazole ring, naphthoxazole ring, oxazolocarbazole ring, oxazole ring, oxazolodibenzofuran ring, pyrrolopyridine ring, pyridine ring, quinoline ring, quinoxaline ring, thiazole ring, or naphthoimidazole ring.

9. The method of claim 1, wherein the near-infrared (NIR) absorbing dye comprises a structure of: 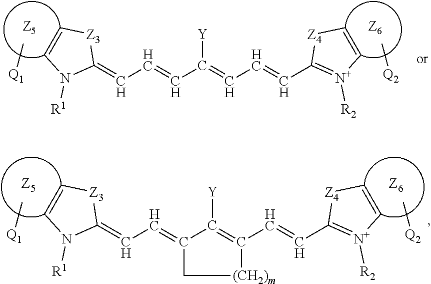
or a rotational or conformational isomer or a salt thereof;
wherein
each of Z₃ and Z₄ is independently —CR¹¹R¹², —NR¹¹, —O—, —S— or —Se—;
each of Z₅ and Z₆ is independently phenyl, naphthyl, pyridinyl, quinolinyl, quinoxalinyl, N-alkyl-benzoindolenine, dibenzofuranyl, or dibenzothiophenyl;
m=1, 2, or 3;
each of R¹¹ and R¹² is independently a C₁₋₆ alkyl; and
Q₁ and Q₂ are independently H, —COOH or a salt thereof, or —SO₃H or a salt thereof.

10. The method of claim 1, wherein the near-infrared (NIR) absorbing dye comprises a structure of: 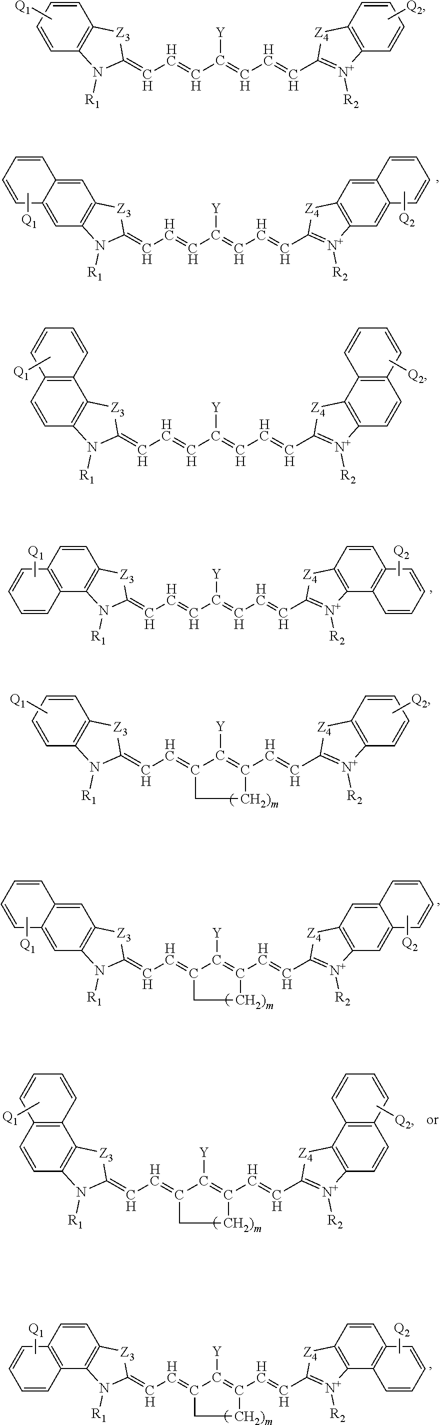
or a rotational or conformational isomer or a salt thereof;
wherein each of Z₃ and Z₄ is independently —CR¹¹R¹², —NR¹¹, —O—, —S— or —Se—;
each of R¹¹ and R¹² is independently a C₁₋₆ alkyl;
m=1, 2, or 3; and
Q₁ and Q₂ are independently H, —COOH or a salt thereof, or —SO₃H or a salt thereof.

11. The method of claim 1, wherein the near-infrared (NIR) absorbing dye comprises a structure of: 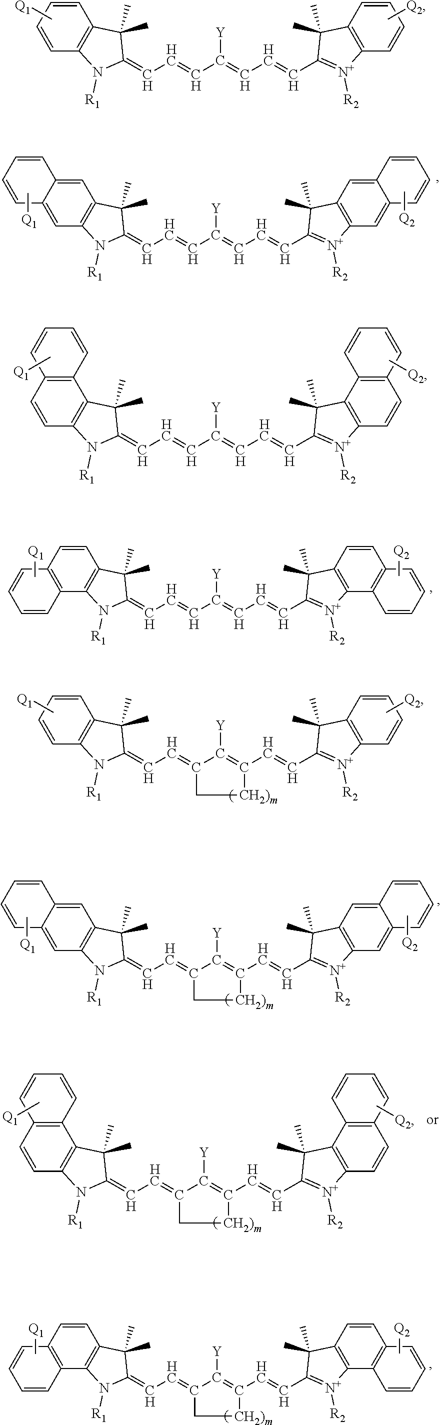
or a rotational or conformational isomer or a salt thereof;
where m=1, 2, or 3; and
R₁ and R₂ are independently —(C₁₋₁₂alkyl)(SO₃)H or a salt thereof or —(C₁₋₁₂alkyl)COOH or a salt thereof.

12. The method of claim 1, wherein the near-infrared (NIR) absorbing dye comprises a structure of either: 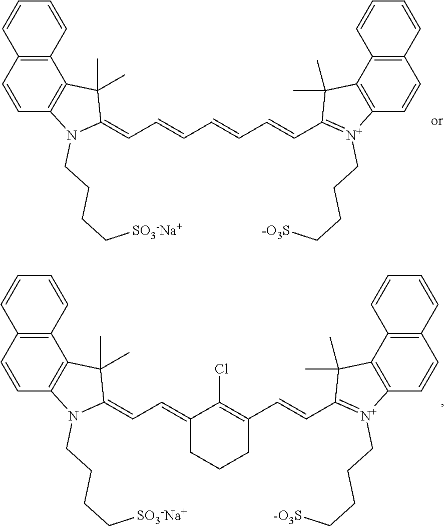
or a rotational or conformational isomer or an alternative salt thereof.

13. A method of altering a mechanical and/or chemical property of a tissue, the method comprising irradiating a near-infrared-(NIR) photoactive direct treatment composition with near-infrared light in the presence of oxygen;
wherein the near-infrared (NIR) photoactive direct treatment composition comprises a near-infrared absorbing dye having a structure of: 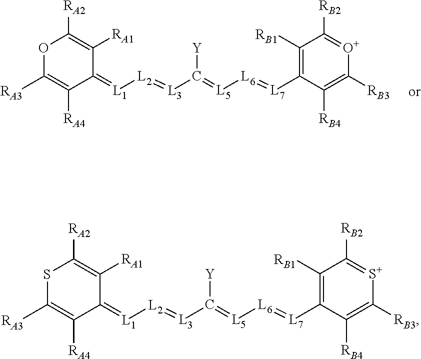
respectively, or a rotational or conformational isomer or a salt thereof; wherein
L₁, L₂, L₃, L₅, L₆, and L₇ are substituted or unsubstituted methines, wherein the optional substituents are independently C₁₋₆ alkyl or C₂₋₆ alkenyl; or L₁ and L₃, or L₃ and L₅, or L₅ and L₇ may be linked with C₂₋₄ alkylene or C₂₋₄ alkenylene substituents;
R_{A1}, R_{A2}, R_{A3}, R_{A4}, R_{B1}, R_{B2}, R_{B3}, and R_{B4} are each independently H, deuterium, or tritium, an C₁₋₁₂ alkyl, —[CH₂—CH₂—O—]₁₋₆R¹⁰, C₂₋₁₂ alkenyl, polyglycol optionally substituted 5- or 10-membered aryl or heteroaryl group, halo, nitro, cyano, —(C₀₋₁₂alkyl) sulfonate or a salt thereof, —(C₀₋₁₂alkyl) sulfate or a salt thereof, —(C₀₋₁₂alkyl)phophate or a salt thereof, —(C₀₋₁₂alkyl)hydroxy, —(C₀₋₁₂alkyl)alkoxy, —(C₀₋₁₂alkyl)aryloxy, —(C₀₋₁₂alkyl)NHSO₃R¹⁰ or a salt thereof, —(C₀₋₁₂alkyl)COOR¹⁰ or a salt thereof, —(C₀₋₁₂alkyl)CON(R¹⁰)₂ or a salt thereof, —(C₀₋₁₂alkyl)N(R¹⁰)₂ or a salt thereof, —(C₀₋₁₂alkyl)borate;
R¹⁰ is independently H or C₁₋₆ alkyl; and
Y is H, or an optionally substituted amine, optionally substituted alkyl, optionally substituted alkoxy, optionally substituted aryl, optionally substituted heteroaryl, optionally substituted aryloxy, optionally substituted heteroaryloxy, halogen, or optionally substituted cationic nitrogen-containing heteroaryl moiety;
that generates singlet oxygen when irradiated with near-infrared light in the presence of oxygen;
wherein the near-infrared (NIR) photoactive direct treatment composition is adjacent to (contacts) or has permeated the tissue; and
wherein the irradiating results in a change in the mechanical and/or chemical property of a tissue.

14. The method of claim 13, wherein the near-infrared (NIR) absorbing dye comprises a structure of: 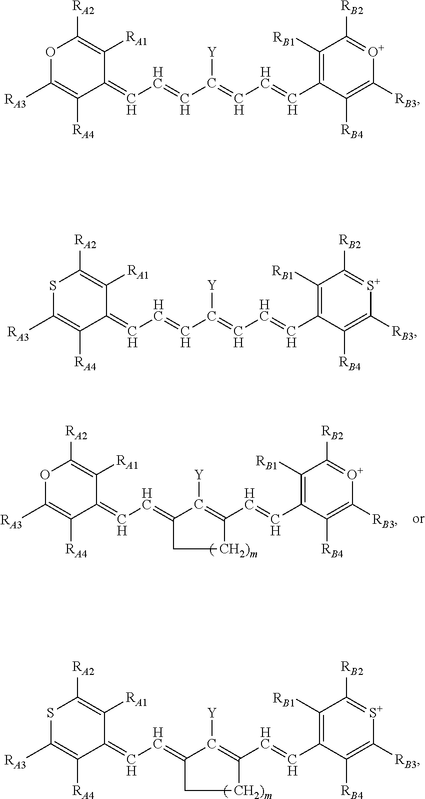
or a rotational or conformational isomer or a salt thereof; where m is 1, 2, or 3.

15. The method of claim 13, wherein R_{A1}, R_{A4}, R_{B1}, and R_{B4} are H, or an isotope thereof, and R_{A2}, R_{A3}, R_{B2}, and R_{B3} are independently aryl, heteroaryl, or branched alkyl.

16. The method of claim 1, wherein Y is H, or an optionally substituted amine, optionally substituted alkyl, optionally substituted alkoxy, optionally substituted aryl, optionally substituted heteroaryl, optionally substituted aryloxy, optionally substituted heteroaryloxy, or halogen.

17. The method of claim 13, wherein Y is H, or an optionally substituted amine, optionally substituted alkyl, optionally substituted alkoxy, optionally substituted aryl, optionally substituted heteroaryl, optionally substituted aryloxy, optionally substituted heteroaryloxy, or halogen.

18. The method of claim 1, wherein Y is an optionally substituted cationic heteroaryl ring moiety and the heptamethine linkage is orthogonally coupled to the optionally substituted cationic heteroaryl ring moiety, preferably comprising:
(a) an optionally substituted acridinium, benzoxazolium, benzothiazolium, imidazolium, isoxazolium, isoquinolinium, isothiazolium, naphthoimidazolium, naphthothiazolium, naphthoxazolium, oxazolium, pyrazinium, pyrazolium, pyridimium, pyridinium, quinolinium, tetrazinium, tetrazolium, thiazolium, triazinium, triazolium, benzopyrazinium, benzopyridimium, benzopyridinium, naphthopyrazinium, naphthopyridimium, benzopyridinium, benzotriazinium, naphthotriazinium moiety, pyrylium, chromenylium, xanthylium moiety, thiopyrylium, thiochromenylium, or thioxanthylium moiety; or
(b) an optionally substituted structure of:

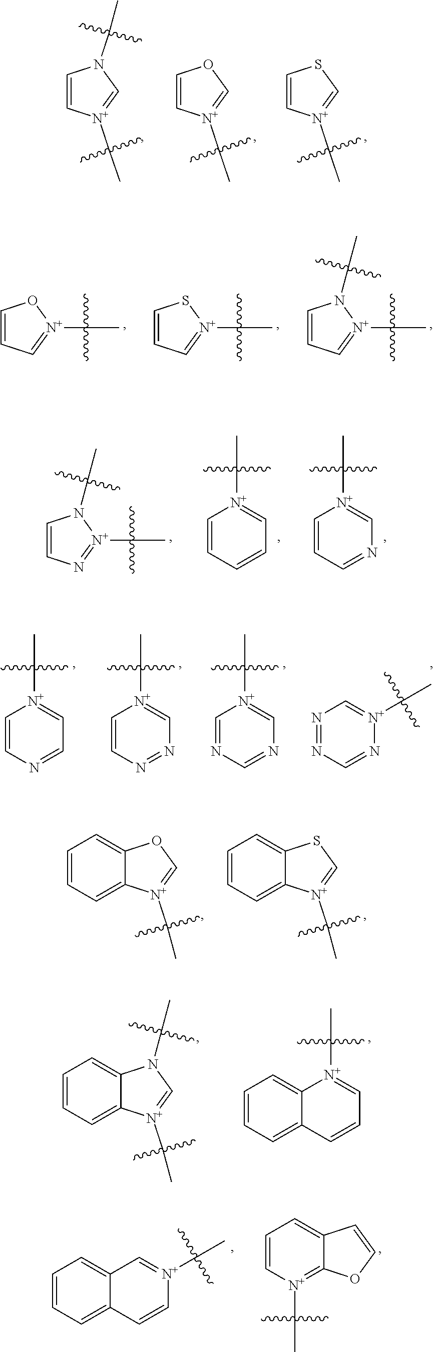

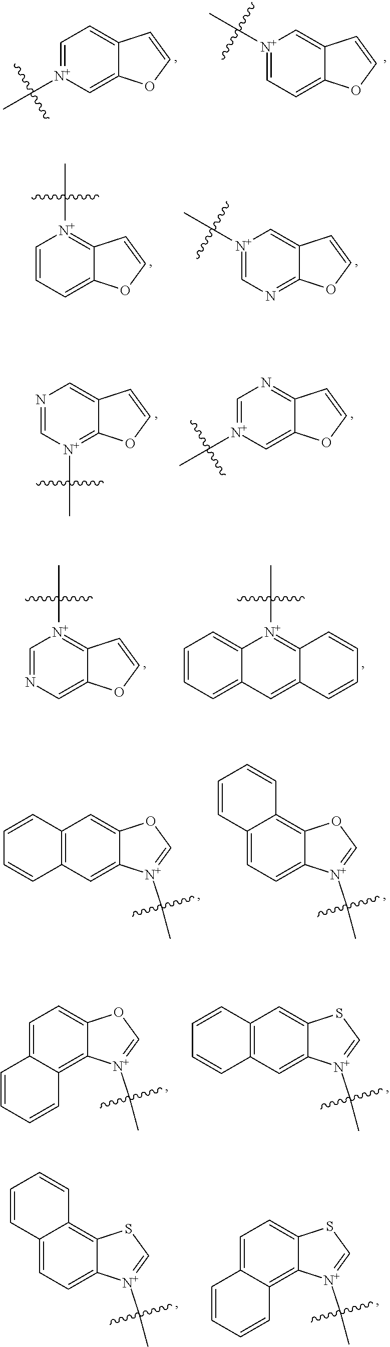

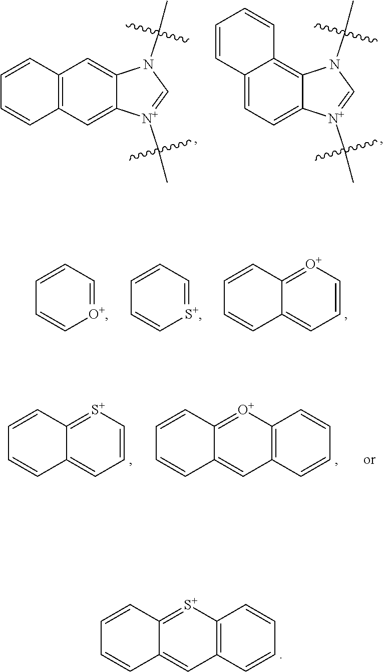

19. The method of claim 13, wherein Y is an optionally substituted cationic heteroaryl ring moiety and the heptamethine linkage is orthogonally coupled to the optionally substituted cationic heteroaryl ring moiety, preferably comprising:
(a) an optionally substituted acridinium, benzoxazolium, benzothiazolium, imidazolium, isoxazolium, isoquinolinium, isothiazolium, naphthoimidazolium, naphthothiazolium, naphthoxazolium, oxazolium, pyrazinium, pyrazolium, pyridimium, pyridinium, quinolinium, tetrazinium, tetrazolium, thiazolium, triazinium, triazolium, benzopyrazinium, benzopyridimium, benzopyridinium, naphthopyrazinium, naphthopyridimium, benzopyridinium, benzotriazinium, naphthotriazinium moiety, pyrylium, chromenylium, xanthylium moiety, thiopyrylium, thiochromenylium, or thioxanthylium moiety; or
(b) an optionally substituted structure of:

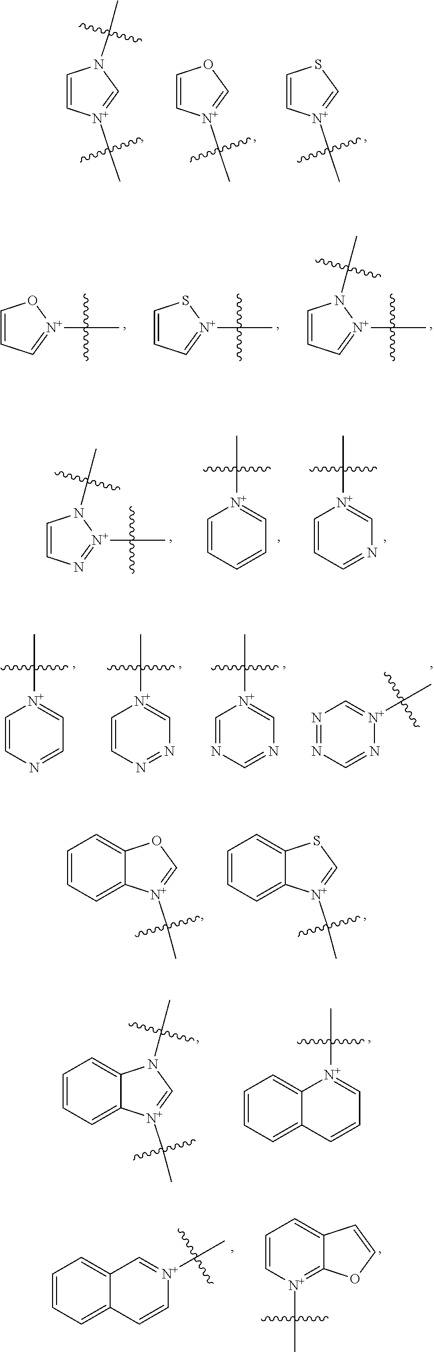

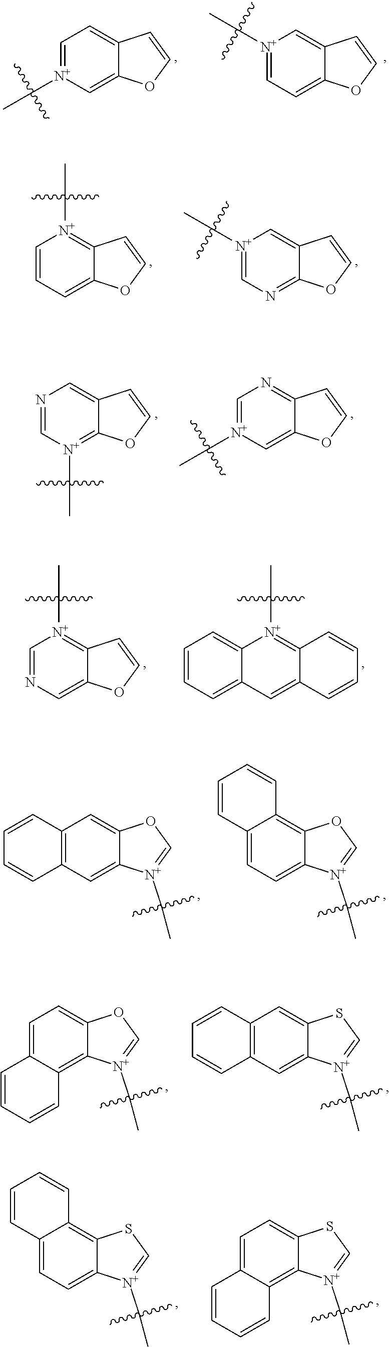

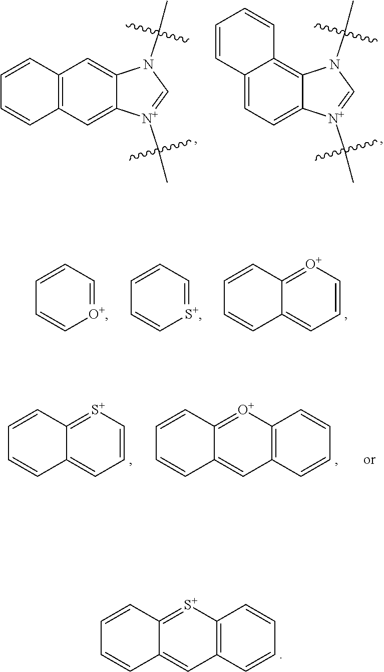

20. The method of claim 1, wherein the irradiating is done with a light having a wavelength in a range of from 800 nm to 1400 nm.

21. The method of claim 1, wherein the near-infrared (NIR) photoactive direct treatment composition further comprises a biocompatible solvent that:
(a) is optically transparent in the UV-VIS and near-infrared range of the optical spectrum;
(b) provides an oxygen solubility greater than H₂O under comparable oxygen partial pressures;
(c) is or comprises a deuterated solvent;
(d) is oxygenated before or during the irradiation; or
(e) a combination of two or more of (a)-(d).

22. The method of claim 1, wherein the near-infrared (NIR) photoactive direct treatment composition further comprises an additive that enhances the solubility of the near-infrared dye.

23. (canceled)

24. The method of claim 1, wherein the near-infrared absorbing dye is indocyanine green: 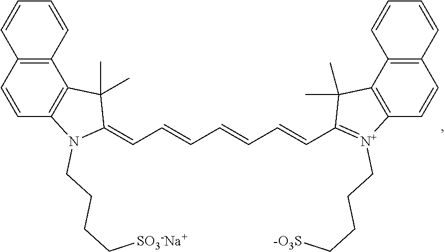
or a structural or conformational isomer thereof;
the tissue includes at least a portion of a sclera; and
the mechanical and/or chemical property is tensile strength, compression strength, flexural strength, modulus, elongation, or toughness of the tissue.

25. The method of claim 1, wherein the tissue is a sclera, and the irradiating results in a strengthening of the sclera, reducing the stretching of the sclera, increasing the modulus of the sclera, reducing the compliance of the sclera, and/or reducing the creep in the sclera.

26. The method of claim 21, wherein the biocompatible solvent is:
(a) a fluorinated or perfluorinated solvent that is optically transparent in the UV-VIS and near-infrared ranges of the optical spectrum and provides an oxygen solubility greater than H₂O under comparable oxygen partial pressures;
(b) is or comprises D₂O; and/or
(c) is oxygenated before or during the irradiation such that the dissolved oxygen is at a level of at least within 50% of the saturation limit of oxygen in the composition.

27. The method of claim 22, wherein the additive that enhances the solubility of the near-infrared dye is:
(a) a surfactant; or
(b) an alkali metal salt; or
(c) independently present at a level of 1 wt % to about 50 wt %, relative to the total weight of the direct treatment composition; or
(d) is a combination of two or more of (a) to (c).
